SEQUENCE LISTING (9 sequences)

SEQ ID NO: 1 - protein, 50 aa, Homo sapiens
MARYRCCRSQSRSRYYRQRQRSRRRRRRSCQTRRRAMRCCRPRYRPRCRR

SEQ ID NO: 2 - protein, 100 aa, Homo sapiens
MVRYRVRSLSERSHEVYRQQLHGQEQGHHGQEEQGLSPEHVEVYERTHGQSHYRRRHCSRRRLHRIHRRQHRSCRRRKRRSCRHRRRHRRGCRTRKRTCR

SEQ ID NO: 3 - RNA, 1653 nt, synthetic construct
  source 1..1653: mol_type = other RNA
atggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtga

SEQ ID NO: 4 - RNA, 1575 nt, Lyssavirus rabies
  source 1..1575: mol_type = mRNA
atggttcctcaggctctcctgtttgtaccccttctggtttttccattgtgttttgggaaattccctatttacacgataccagacaagcttggtccctggagcccgattgacatacatcacctcagctgcccaaacaatttggtagtggaggacgaaggatgcaccaacctgtcagggttctcctacatggaacttaaagttggatacatctcagccataaaaatgaacgggttcacttgcacaggcgttgtgacggaggctgaaacctacactaacttcgttggttatgtcacaaccacgttcaaaagaaagcatttccgcccaacaccagatgcatgtagagccgcgtacaactggaagatggccggtgaccccagatatgaagagtctctacacaatccgtaccctgactaccactggcttcgaactgtaaaaaccaccaaggagtctctcgttatcatatctccaagtgtggcagatttggacccatatgacagatcccttcactcgagggtcttccctggcgggaattgctcaggagtagcggtgtcttctacctactgctccactaaccacgattacaccatttggatgcccgagaatccgagactagggatgtcttgtgacatttttaccaatagtagagggaagagagcatccaaagggagtgagacttgcggctttgtagatgaaagaggcctatataagtctttaaaaggagcatgcaaactcaagttatgtggagttctaggacttagacttatggatggaacatgggtcgcgatgcaaacatcaaatgaaaccaaatggtgccctcccggtcagttggtgaatttgcacgactttcgctcagacgaaattgagcaccttgttgtagaggagttggtcaagaagagagaggagtgtctggatgcactagagtccatcatgaccaccaagtcagtgagtttcagacgtctcagtcatttaagaaaacttgtccctgggtttggaaaagcatataccatattcaacaagaccttgatggaagccgatgctcactacaagtcagtcagaacttggaatgagatcatcccttcaaaagggtgtttaagagttggggggaggtgtcatcctcatgtaaacggggtatttttcaatggtataatattaggacctgacggcaatgtcttaatcccagagatgcaatcatccctcctccagcaacatatggagttgttggtatcctcggttatcccccttatgcaccccctggcagacccgtctaccgttttcaagaacggtgacgaggctgaggattttgttgaagttcaccttcccgatgtgcacgaacggatctcaggagttgacttgggtctcccgaactgggggaagtatgtattactgagtgcaggggccctgactgccttgatgttgataattttcctgatgacatgctggagaagagtcaatcgatcggaacctacacaacacaatctcagagggacagggagggaggtgtcagtcactccccaaagcgggaagatcatatcttcatgggaatcatacaagagcgggggtgagaccggactgtga

SEQ ID NO: 5 - RNA, 1575 nt, synthetic construct
  source 1..1575: mol_type = other RNA
atggttcctcaggctctgctgttcgtgcccctgctggtgttccctctgtgcttcggcaagttccccatctacacaatccccgacaagctcggcccttggagccctatcgatatccaccacctgagctgccccaacaacctggtggtggaagatgagggctgcaccaacctgagcggcttcagctacatggaactgaaagtgggctacatcagcgccatcaagatgaacggcttcacctgtaccggcgtggtcacagaggccgagacatacacaaacttcgtgggctacgtgaccaccaccttcaagagaaagcacttcagacccacacctgacgcctgcagagccgcctacaattggaagatggccggcgatcccagatacgaggaaagcctgcacaacccctatcctgactaccactggctgagaaccgtgaaaaccaccaaagagagcctggtcatcatcagccccagcgtggcagacctggatccttacgacagatccctgcacagcagagtgttccctggcggcaactgtagcggagtggctgtgtctagcacctactgcagcaccaaccacgactacaccatctggatgcctgagaaccccagactgggcatgagctgcgacatcttcaccaactccagaggcaagagagccagcaagggcagcgagacatgtggcttcgtggatgagagaggcctgtacaagtctctgaagggcgcctgcaagctgaagctgtgtggtgtcctgggcctgagactgatggatggaacatgggtcgccatgcagaccagcaacgagacaaagtggtgtccacctggccagctggtcaacctgcacgactttagaagcgacgagatcgagcatctggtggtcgaggaactggtcaagaaaagggaagagtgcctggacgccctggaatccatcatgaccaccaagagcgtgtccttcagaaggctgagccacctgagaaagctggtgcccggctttggcaaggcctataccatcttcaacaagaccctgatggaagccgacgctcactacaagtctgtgcggacctggaacgagatcatccctagcaagggctgcctgagagtcggcggaagatgtcaccctcatgtgaacggcgtgttcttcaacggcatcatcctgggacctgacggcaacgtgctgatccctgagatgcagtctagcctgctccagcagcatatggaactgctcgtgtccagcgtgatccctctgatgcaccctctggccgatcctagcaccgtgttcaagaatggcgacgaggccgaggacttcgtggaagtgcatctgcctgacgtgcacgagagaatctctggcgttgacctgggactgcccaactggggcaaatacgtgctgctttctgccggcgctctgacagccctgatgctgatcatcttcctgatgacctgctggcggagagtgaacagaagcgagcccacacagcacaacctgagaggcacaggcagagaagtgtctgtgacaccccagagcggcaagatcatcagcagctgggagagctacaagagcggcggagagacaggactgtga

SEQ ID NO: 6 - RNA, 1698 nt, Alphainfluenzavirus
  source 1..1698: mol_type = mRNA
atgaaggcaaacctactggtcctgttatgtgcacttgcagctgcagatgcagacacaatatgtataggctaccatgcgaacaattcaaccgacactgttgacacagtactcgagaagaatgtgacagtgacacactctgttaacctgctcgaagacagccacaacggaaaactatgtagattaaaaggaatagccccactacaattggggaaatgtaacatcgccggatggctcttgggaaacccagaatgcgacccactgcttccagtgagatcatggtcctacattgtagaaacaccaaactctgagaatggaatatgttatccaggagatttcatcgactatgaggagctgagggagcaattgagctcagtgtcatcattcgaaagattcgaaatatttcccaaagaaagctcatggcccaaccacaacacaaacggagtaacggcagcatgctcccatgaggggaaaagcagtttttacagaaatttgctatggctgacggagaaggagggctcatacccaaagctgaaaaattcttatgtgaacaaaaaagggaaagaagtccttgtactgtggggtattcatcacccgcctaacagtaaggaacaacagaatctctatcagaatgaaaatgcttatgtctctgtagtgacttcaaattataacaggagatttaccccggaaatagcagaaagacccaaagtaagagatcaagctgggaggatgaactattactggaccttgctaaaacccggagacacaataatatttgaggcaaatggaaatctaatagcaccaatgtatgctttcgcactgagtagaggctttgggtccggcatcatcacctcaaacgcatcaatgcatgagtgtaacacgaagtgtcaaacacccctgggagctataaacagcagtctcccttaccagaatatacacccagtcacaataggagagtgcccaaaatacgtcaggagtgccaaattgaggatggttacaggactaaggaacaatccgtccattcaatccagaggtctatttggagccattgccggttttattgaagggggatggactggaatgatagatggatggtatggttatcatcatcagaatgaacagggatcaggctatgcagcggatcaaaaaagcacacaaaatgccattaacgggattacaaacaaggtgaacactgttatcgagaaaatgaacattcaattcacagctgtgggtaaagaattcaacaaattagaaaaaaggatggaaaatttaaataaaaaagttgatgatggatttctggacatttggacatataatgcagaattgttagttctactggaaaatgaaaggactctggatttccatgactcaaatgtgaagaatctgtatgagaaagtaaaaagccaattaaagaataatgccaaagaaatcggaaatggatgttttgagttctaccacaagtgtgacaatgaatgcatggaaagtgtaagaaatgggacttatgattatcccaaatattcagaagagtcaaagttgaacagggaaaaggtagatggagtgaaattggaatcaatggggatctatcagattctggcgatctactcaactgtcgccagttcactggtgcttttggtctccctgggggcaatcagtttctggatgtgttctaatggatctttgcagtgcagaatatgcatctga

SEQ ID NO: 7 - RNA, 1704 nt, Alphainfluenzavirus
  source 1..1704: mol_type = mRNA
atgaagactatcattgctttgagctacattttctgtctggttttcgcccaagaccttccaggaaatgacaacaacagcacagcaacgctgtgcctgggacatcatgcggtgccaaacggaacgctagtgaaaacaatcacgaatgatcagattgaagtgactaatgctactgagctggttcagagttcctcaacgggtaaaatatgcaacaatcctcatcgaatccttgatggaataaactgcacactgatagatgctctattgggggaccctcattgtgatggatttcaaaatgagaaatgggaccttttcgttgaacgcagcaaagctttcagcaactgttacccttatgatgtgccagattatgcctcccttaggtcactagttgcctcgtcaggcactctggagtttatcaatgaaggcttcaattggactggggtcactcagaatgggggaagcagtgcttgcaaaagaggacctgatagcggttttttcagtagactgaactggttgtacaaatcaggaagcacatatccagtgcaaaacgtgaccatgccaaacaatgacaattctgacaaactatacatttggggggttcaccacccgagcacggacaaagaacaaaccaacctatatgttcaagcatcagggaaagtcacagtctccaccaagagaagccagcaaactataatcccgaatgtcgggtctagaccctgggtaaggggtctgtctagtagaataagcatctattggacaatagttaaaccgggagacatactggtaattaatagtaatgggaacctaattgctcctcggggttacttcaaaatgcgcactgggaaaagctcaataatgaggtcagatgcacctattggcacctgcagctctgaatgcatcactccaaatggaagcattcccaatgacaagccctttcaaaacgtaaacaagatcacatatggggcatgtcccaagtatgttaagcaaaacactctgaagttggcaacagggatgcggaatgtaccagagaaacaaactagaggcatattcggcgcaatagcaggtttcatagaaaatggttgggagggaatgatagacggttggtacggtttcaggcatcaaaattccgagggcacaggacaagcagcagatcttaaaagcactcaagcagccatcgaccaaatcaatgggaaactgaatagggtaatcgagaagacgaacgagaaattccatcaaatcgaaaaggaattctcagaagtagaagggagaattcaggacctcgagaaatacgttgaagacactaaaatagatctctggtcttacaatgcggagcttcttgtcgctctggagaaccaacatacaattgatctgactgactcggaaatgaacaaactgtttgaaaaaacaaggaggcaactgagggaaaatgctgaggacatgggcaatggttgcttcaaaatataccacaaatgtgacaatgcttgcatagggtcaatcagaaatggtacttatgaccatgatgtatacagagacgaagcattaaacaaccggtttcagatcaaaggtgttgaactgaagtcaggatacaaagactggatcctgtggatttcctttgccatatcatgctttttgctttgtgttgttttgctggggttcatcatgtgggcctgccaaaaaggcaacattaggtgcaacatttgcatttga

SEQ ID NO: 8 - RNA, 1755 nt, Betainfluenzavirus
  source 1..1755: mol_type = mRNA
atgaaggcaataattgtactactcatggtagtaacatccaatgcagatcgaatctgcactggaataacatcttcaaactcacctcatgtggtcaaaacagccactcaaggggaggtcaatgtgactggtgtgataccactaacaacaacaccaacaaaatcttattttgcaaatctcaaaggaacaaggaccagagggaaactatgcccagactgtctcaactgcacagatctggatgtggctttgggcagaccaatgtgtgtggggaccacaccttcggcgaaagcttcaatactccacgaagtcaaacctgttacatccgggtgctttcctataatgcacgacagaacaaaaatcaggcaactacccaatcttctcagaggatatgaaaatatcaggctatcaacccaaaacgtcatcgatgcggaaaaggcaccaggaggaccctacagacttggaacctcaggatcttgccctaacgctaccagtaagagcggatttttcgcaacaatggcttgggctgtcccaaaggacaacaacaaaaatgcaacgaacccactaacagtagaagtaccatacatttgtacagaaggggaagaccaaatcactgtttgggggttccattcagatgacaaaacccaaatgaagaacctctatggagactcaaatcctcaaaagttcacctcatctgctaatggagtaaccacacactatgtttctcagattggcagcttcccagatcaaacagaagacggaggactaccacaaagcggcaggattgttgttgattacatgatgcaaaaacctgggaaaacaggaacaattgtctaccaaagaggtgttttgttgcctcaaaaggtgtggtgcgcgagtggcaggagcaaagtaataaaagggtccttgcctttaattggtgaagcagattgccttcatgaaaaatacggtggattaaacaaaagcaagccttactacacaggagaacatgcaaaagccataggaaattgcccaatatgggtgaaaacacctttgaagcttgccaatggaaccaaatatagacctcctgcaaaactattaaaggaaaggggtttcttcggagctattgctggtttcctagaaggaggatgggaaggaatgattgcaggctggcacggatacacatctcacggagcacatggagtggcagtggcggcggaccttaagagtacgcaagaagctataaacaagataacaaaaaatctcaattctttgagtgagctagaagtaaagaatcttcaaagactaagtggtgccatggatgaactccacaacgaaatactcgagctggatgagaaagtggatgatctcagagctgacactataagctcgcaaatagaacttgcagtcttgctttccaacgaaggaataataaacagtgaagatgagcatctattggcacttgagagaaaactaaagaaaatgctgggtccctctgctgtagagataggaaatggatgcttcgaaaccaaacacaagtgcaaccagacctgcttagacaggatagctgctggcacctttaatgcaggagaattttctctccccacttttgattcactgaacattactgctgcatctttaaatgatgatggattggataaccatactatactgctctattactcaactgctgcttctagtttggctgtaacattgatgctagctatttttattgtttatatggtctccagagacaacgtttcatgctccatctgtctataa

SEQ ID NO: 9 - RNA, 1758 nt, Betainfluenzavirus
  source 1..1758: mol_type = mRNA
atgaaggcaataattgtactactcatggtagtaacatccaatgcagatcgaatctgcactgggataacatcgtcaaactcaccccatgtggtcaaaactgctactcaaggggaagtcaatgtgactggtgtgataccactgacaacaacaccaaccaaatctcattttgcaaatctcaaaggaacaaaaaccagagggaaactatgcccaaagtgtctcaactgcacagatctggacgtggccttggcgagaccaaagtgcatggggaccataccttcggcaaaagcttcaatactccatgaagtcaaacctgttacatctgggtgctttcctataatgcacgacagaacaaaaattagacagctacccaatcttctcagaggatacgaacatatcaggttatcaacccataacgttatcaacgcagaaacggcaccaggaggaccctacaaagttggaacctcagggtcttgccctaacgttaccaatggaaacggattcttcgcaacaatggcttgggctgtcccaaaaaacgacaacaacaaaacagcaacaaatccattaacagtagaagtaccatacatttgtacagaaggagaagaccaaattactgtttgggggttccactctgatagcgaaacccaaatggtaaaactctatggagactcaaagcctcagaagttcacctcatctgccaatggagtaaccacacattacgtttcacagattggtggcttcccaaatcaagcagaagacggagggctaccacaaagcggtagaattgttgttgattacatggtgcaaaaatctggaaaaacaggaacaattacctaccaaagaggtattttattgcctcaaaaagtgtggtgcgcaagtggcaggagcaaggtaataaaagggtccttgcctttaattggtgaagcagattgcctccacgaaaaatacggtggattaaacaaaagcaagccttactacacaggagaacatgcaaaagccataggaaattgcccaatatgggtgaaaacacccttgaagctggccaatggaaccaaatatagacctcctgcaaaactattaaaggaaaggggtttcttcggagctattgctggtttcttagaaggaggatgggaaggaatgattgcaggttggcacggatacacatcccatggagcacatggagtagcagtggcagcagaccttaagagtacgcaagaagccataaacaagataacaaaaaatctcaattctttgagtgagctggaagtaaagaatcttcaaagactaagcggtgccatggatgaactccacaacgaaatactcgaactggatgagaaagtggatgatctcagagctgatacaataagctcgcaaatagagctcgcagtcttgctttccaacgaaggaataataaacagtgaagatgagcatctcttggcgcttgaaagaaaactgaagaaaatgctgggcccctctgctgtagagatagggaatggatgcttcgaaaccaaacacaagtgcaaccagacctgcctagacagaatagctgctggcacctttaatgcaggagaattttctcttcccacctttgattcactaaatattactgctgcatctttaaatgatgatggattggataatcatactatactgctttactactcaactgctgcttccagtttggctgtaacattgatgatagctatctttattgtttatatggtctccagagacaatgtttcttgctccatctgtctatag

(19) United States
(12) Patent Application Publication
(10) Pub. No.: US 2023/0372475 A1
(43) Pub. Date: Nov. 23, 2023

(54) STABILIZATION OF ANTIGENS FOR LONG TERM ADMINISTRATION IN TRANSDERMAL MICRONEEDLE PATCHES

(71) Applicants: UNIVERSITY OF CONNECTICUT, FARMINGTON, CT (US); EASTERN VIRGINIA MEDICAL SCHOOL, NORFOLK, VA (US)

(72) Inventors: Vivek Agrahari, Oklahoma City, OK (US); Gustavo Fabian Doncel, Norfolk, VA (US); Thanh Duc Nguyen, Farmington, CT (US); Feng Lin, Farmington, CT (US); Khanh Tran, Farmington, CT (US); Onkar Nath Singh, Fort Worth, TX (US)

(21) Appl. No.: 18/247,962

(22) PCT Filed: Sep. 2, 2022
(86) PCT No.: PCT/US2022/075888

§ 371 Date: Apr. 5, 2023

Related U.S. Application Data
(60) Provisional application No. 63/240,743, filed on Sep. 3, 2021.
(60) Provisional application No. 63/329,721, filed on Apr. 11, 2022.

(51) Int. Cl. A61K 39/39; A61K 9/127; A61K 47/54; A61K 47/26; A61K 39/395; A61K 39/215; A61K 9/00

(52) U.S. Cl. CPC A61K 39/39; A61K 9/1272; A61K 47/543; A61K 47/26; A61K 39/39591; A61K 39/215; A61K 9/0021; A61K 2039/53

(57) ABSTRACT

Described herein are compositions and methods for stabilizing RNA and protein antigens for long-term storage and use in transdermal microneedle patches, methods for filling microneedles, and methods of use. A stabilized RNA vaccine composition comprises: a complex of RNA with one or more cationic polymers; and one or more cationic lipid entities. A method for stabilizing RNA comprises: forming a complex comprising the RNA with one or more cationic polymers; mixing the complex with one or more cationic lipid entities comprising liposomes or lipid nanoparticles to form a lipid mixture; and drying the lipid mixture under vacuum. The compositions and methods may be employed in the preparation of vaccine medicaments.

### CROSS-REFERENCE TO RELATED APPLICATIONS

This application claims priority to U.S. Provisional Patent Application Nos. 63/240,743, filed on Sep. 3, 2021, and 63/329,721, filed on Apr. 11, 2022, each of which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “209670-9057-WO01_sequence_listing_xml_22-AUG-2022.xml,” was created on Aug. 22, 2022, contains 9 sequences, has a file size of 20.0 Kbytes, and is incorporated by reference in its entirety into the specification.

### TECHNICAL FIELD

Described herein are compositions and methods for stabilizing RNA and protein antigens for long-term storage and use in transdermal microneedle patches, methods for filling microneedles, and methods of use.

### BACKGROUND

Vaccines often require repeated injections (i.e., a prime-boost-boost regimen) to offer full immune protection. These multiple injections are painful, expensive, and inconvenient, causing low patient compliance/adherence and significant logistical issues. RNA-based therapeutics, including mRNA vaccines (e.g., mRNA vaccines against COVID-19), also require multiple injections which are painful, inconvenient, and costly. Furthermore, these mRNAs suffer from a significant problem of thermal instability, which requires the vaccines to be stored in stringent cold environments (i.e., about −80° C.). Recent works of formulating mRNA inside lipid nanoparticles (LNPs) or complexing mRNA with stabilizing agents (e.g., protamine) in aqueous form have shown that the mRNA can be stabilized against harsh temperature and storage conditions for up to months. However, the immune response to these mRNA complexes is dose-dependent and varies with administration routes.

It was previously reported that the use of conventional intramuscular injections with syringes/needles did not provide good response to these mRNA complexes as compared to what was obtained from injection-free intradermal devices. It is hypothesized that this is most likely due to the lack of immune cells in the deep intramuscular area (compared to dermal areas) to uptake and translate the stabilized mRNA complex. Furthermore, it is thought that mRNA stability may be enhanced by formulating the mRNA in a dry form with an assistance of excipients such as using polyol (e.g., —OH contained excipients such as trehalose, sucrose, mannitol, silk, etc.) to create a highly viscous environment during the drying process that protects the dried mRNA. However, to date, no one has been successful in stabilizing mRNA at room or body temperature for long-term. While there has been some achievement for formulating the mRNA with protamine, as mentioned above, the reported transfection efficiency has not been consistent and relies on a bulky device for transdermal delivery to enhance the efficiency of the mRNA vaccine.

An injection-free vaccine microneedle (MN) patch (similar to a nicotine patch) offers an excellent intradermal delivery system which is easy-to-use and could be self-administered by patients at homes. Different from the conventional subcutaneous or intramuscular hypodermic injections, the MNs target the superficial dermal layer on top of the skin where there are many dendritic Langerhan cells that are beneficial to uptake and translation of the mRNA to enhance immunogenicity. Indeed, previous research has shown that when the influenza vaccine is delivered by intradermal MNs, it offers 3- or 4-fold increases of neutralizing antibody titers, compared to the use of subcutaneous injection of the same vaccine dose. However, most available MN patches that have been developed so far (e.g., Nanopatch™) rely on simple methods to coat or mix vaccines onto or within the polymeric MNs, and thus only provide immediate release (i.e., burst release at time 0). This limitation of current MNs requires repeated patch applications over a long period to mimic the booster vaccine shots. This causes significant burdens on how to store the MN patches appropriately (i.e., using cold temperatures to avoid thermal instability of vaccines) and sustain the vaccine bioactivity for follow-up patch applications, and also how to make patients remember the vaccine schedule to perform the other booster administrations at precise times.

Furthermore, during large scale outbreaks of highly infectious diseases, such as the COVID-19 pandemic caused by the virus SARS-CoV-2, widespread vaccination is often utilized to achieve community immunity at a global scale in order to prevent transmission and potential mutation of the pathogens, associated diseases, and loss of life. However, traditional vaccination methods relying on subcutaneous or intramuscular injections suffer from logistical burdens which hinder their rapid deployment on a massive scale. First, the injections are uncomfortable, painful, and require trained professionals to administer the doses. Secondly, extensive research works have shown that vaccines often require booster shots to sustain an effective antibody level and long-term immune protections. These booster shots are repeated over weeks or months which not only increase logistic burdens but also could be a challenge for patients to adhere to the schedules. Additionally, as patients need to travel to hospitals or medical centers for access to vaccinations, booster appointments during outbreaks can increase the risk of cross infection and the burden on healthcare facilities already stressed by increased caseloads. In developing countries, these hurdles can be even more problematic as many patients are living in remote areas and cannot afford repeated travels for multiple booster shots. These substantial problems of low-patient compliance and adherence pose significant challenges to global vaccination campaign against SARS-CoV-2, polio, measle viruses etc. Thirdly, many vaccines have to be consistently stored under stringent conditions with very low temperatures. This cold-chain requirement presents a significant obstacle to the dissemination of necessary vaccines in lower-income or resource-disadvantaged communities due to the need for extended storage of vaccines for priming and booster shots. Collectively, (1) the painful, and inconvenient nature of needle-based injections which reduce patient compliance, (2) the need of booster injections, and (3) the requirement of cold-chain storage are critical problems of current vaccines which need to be overcome in order to address the current COVI D-19 crisis, increase vaccine coverage worldwide, and prepare us for potential future outbreaks.

What is needed are easier-to-use, minimally invasive, and more versatile intradermal vaccine delivery systems that can optimize the immune efficacy and fully empower thermal stability of complexed mRNA vaccines and protein antigen vaccines.

### SUMMARY

One embodiment described herein is a stabilized RNA vaccine composition, the composition comprising: a complex of RNA with one or more cationic polymers; and one or more cationic lipid entities. In one aspect, the RNA comprises mRNA, miRNA, siRNA, tRNA, rRNA, or other RNA. In another aspect, the one or more cationic polymers comprise one or more of protamine, poly-lysine, poly-arginine, poly-histidine, amino group modified polycarbonate, amino group modified polypeptide-, poly-β-amino esters, cationic cellulose, cationic dextran, cationic cyclodextrin, or combinations thereof. In another aspect, the cationic polymer comprises protamine. In another aspect, the one or more cationic lipid entities comprises liposomes or lipid nanoparticles. In another aspect, the liposomes or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammoniurn propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof. In another aspect, the composition further comprises one or more cryoprotectants. In another aspect, the one or more cryoprotectants comprises one or more of trehalose, mannitol, sucrose, glucose, fructose, lactose, dextran, glycerol, ethylene glycol, propylene glycol, polyethylene glycol, or combinations thereof. In another aspect, the composition further comprises one or more amino acids selected from lysine, arginine, histidine, glutamine, praline, glycine, threonine, or combinations thereof. In another aspect, the complex of RNA with one or more cationic peptides has a size ranging from about 20 nm to about 500 nm. In another aspect, the cationic lipid entity has a size ranging from about 20 nm to about 500 nm. In another aspect, the stabilized RNA is thermally stable for at least about 2 months at about 37° C. In another aspect, the stabilized RNA is thermally stable for at least about 2 hours at about 80° C.

Another embodiment described herein is method for stabilizing RNA, the method comprising: (a) forming a complex comprising the RNA with one or more cationic polymers; (b) mixing the complex with one or more cationic lipid entities comprising liposomes or lipid nanoparticles to form a lipid mixture; and (c) drying the lipid mixture under vacuum. In one aspect, the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof. In another aspect, the cationic polymer comprises protamine. In another aspect, the liposome or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof. In another aspect, the method further comprises mixing the lipid mixture with one or more cryoprotectants comprising one or more of, trehalose, mannitol, sucrose, glucose, fructose, lactose, dextran, glycerol, ethylene glycol, propylene glycol, polyethylene glycol, or combinations thereof prior to drying the lipid mixture. In another aspect, the method further comprises mixing the lipid mixture with one or more amino acids selected from lysine, arginine, histidine, glutamine, proline, glycine, threonine, or combinations thereof prior to drying the lipid mixture.

Another embodiment described herein is a stabilized RNA vaccine prepared by the methods described herein.

Another embodiment described herein is the use of a stabilized RNA prepared by the methods described herein in the preparation of a vaccine medicament.

Another embodiment described herein is a stabilized vaccine agent composition, the composition comprising: a vaccine agent; and one or more cryoprotectants comprising one or more of trehalose, mannitol, sucrose, glucose, fructose, lactose, dextran, glycerol, ethylene glycol, propylene glycol, polyethylene glycol, or combinations. In one aspect, the one or more cryoprotectants have a concentration of about 0.3 M to about 1 M. In another aspect, the vaccine agent and the one or more cryoprotectants are present in a ratio ranging from about 1:1 (v/v) to about 1:6 (v/v). In another aspect, the vaccine agent is a protein, functional fragment thereof, or combination thereof. In another aspect, the composition is dried by lyophilization, vacuum, or dessication. In another aspect, the vaccine agent is thermally stable for at least about 4 months at about 37° C. In another aspect, the vaccine agent is thermally stable for at least about 1 hour at about 100° C.

Another embodiment described herein is a method for preparing a stabilized vaccine agent core, the method comprising: (a) mixing a vaccine agent with one or more cryoprotectants comprising one or more of trehalose, mannitol, sucrose, glucose, fructose, lactose, dextran, glycerol, ethylene glycol, propylene glycol, polyethylene glycol, or combinations thereof; and (b) drying the mixture. In one aspect, the one or more cryoprotectants have a concentration of about 0.3 M to about 1 M prior to drying. In one aspect, the vaccine agent is mixed with the one or more cryoprotectants at a ratio ranging from about 1:1 (v/v) to about 1:6 (v/v) prior to drying. In another aspect, the vaccine agent is a protein, functional fragment thereof, or combination thereof. In another aspect, drying in step (b) is performed by lyophilization, vacuum, or dessication. In another aspect, drying in step (b) is performed for at least about 4 hours to about 24 hours.

Another embodiment described herein is a stabilized vaccine core prepared by the methods described herein.

Another embodiment described herein is the use of a stabilized vaccine core prepared by the methods described herein the preparation of a vaccine medicament.

Another embodiment described herein is a method for one-step loading a vaccine core into a microneedle, the method comprising: (a) generating a core-shell microneedle assembly comprising a polymeric shell by compression-molding a film of poly(D,L-lactide-co-glycolide) (PLGA) into a silicone or poly(dimethylsiloxane) (PDMS) mold to form an assembly of microneedle shells, wherein the mold has an array of conical shaped cavities; (b) generating a vaccine core array by combining a vaccine agent with a cryoprotectant and casting the vaccine core on a second poly(dimethylsiloxane) (PDMS) mold having the same array of conical shaped cavities as the microneedle mold and drying the vaccine core; (c) transferring the vaccine core array onto a PLGA film; (d) aligning the vaccine core array on the PLGA film with the microneedle assembly, thereby filling the cores in the microneedle shell assembly with the vaccine cores; and (e) curing the microneedle assembly by vacuum heating to encapsulate the vaccine cores within the microneedles. In one aspect, generating a core-shell microneedle structure further comprises: removing an excess PLGA scum layer. In another aspect, the microneedles have a height of about 600 μm and a base diameter of about 300 μm. In another aspect, the vaccine cores have a height of about 400 μm and a diameter of about 200 μm.

Another embodiment described herein is a one-step loaded microneedle manufactured by the methods described herein.

Another embodiment described herein is the use of a one-step loaded microneedle manufactured by the methods described herein to deliver a vaccine to a subject in need thereof.

Another embodiment described herein is a method for powder-filling a microneedle assembly, the method comprising: (a) preparing a poly(dimethylsiloxane) (PDMS) mold having an array of conical shaped cavities on a silicon wafer; (b) dispersing a powdered vaccine agent into the mold cavities; (c) compressing the powdered vaccine agent into the mold cavities; (d) repeating steps (b) and (c) until the microneedle cavities are filled; (e) casting a layer of one or more sugars and water-soluble polymer over the filled cavities to generate a microneedle assembly; and (f) drying the microneedle assembly. In one aspect, steps (b)-(d) are performed in a neutralized static environment. In another aspect, the vaccine agent loading capacity is about 20-fold greater than solution-casting core filling. In another aspect, the vaccine agent is a protein, a nucleic acid, or a combination thereof.

Another embodiment described herein is a filled microneedle assembly manufactured by the methods described herein.

Another embodiment described herein is use of a filled microneedle assembly manufactured by the methods described herein to deliver a vaccine to a subject in need thereof.

Another embodiment described herein is a method for manufacturing a microneedle assembly, the method comprising: (a) generating a core microneedle assembly by: filling a first silicone mold including a plurality of microneedle cavities with a copolymer, spinning the first silicone mold to remove a first scum layer of the copolymer on the first silicone mold, and generating a core in each of the copolymer-filled cavities; (b) generating a therapeutic agent assembly by: filling a plurality of cavities in a second silicone mold with a therapeutic agent, spinning the second silicone mold to remove a second scum layer of the therapeutic agent, and removing the molded therapeutic agent from the second mold and transferring the molded therapeutic agent to a substrate; and (c) aligning the therapeutic agent assembly with the core microneedle assembly to thereby fill the cores in the core microneedle assembly with the therapeutic agent. In one aspect, the cavities in the first silicone mold and the second silicone mold are conical shaped. In another aspect, the copolymer is poly(D,L-lactide-co-glycolide) (PLGA). In another aspect, the method further comprises generating the first silicone mold by pouring polydimethylsiloxane (PDMS) over a master structure and curing. In another aspect, the vaccine agent is a protein or a nucleic acid. In another aspect, the method further comprises generating a cap assembly by: (a) filling a plurality of cavities in a third silicone mold with a copolymer to form a plurality of caps; (b) spinning the third silicone mold to remove a third scum layer of the copolymer on the third silicone mold; (c) removing the molded caps from the third mold and transferring the molded caps onto the core microneedle assembly; and (d) aligning the cap assembly with the core microneedle assembly to thereby cover the cores in the core microneedle assembly with the caps. In another aspect, the method further comprises applying heat to bond the cap assembly to the core microneedle assembly. In another aspect, the method further comprises coating a supporting array with a water-soluble polymer and contacting the supporting array with the cap assembly. In another aspect, the method further comprises applying heat to bond the supporting array to the cap assembly for removing the microneedle assembly from the first silicone mold.

Another embodiment described herein is microneedle device, manufactured by the methods described herein.

Another embodiment described herein is the use of a microneedle device, manufactured by the methods described herein for the delivery of a vaccine to a subject in need thereof.

Another embodiment described herein is a pulsatile vaccine delivery system comprising: a microneedle assembly including a plurality of microneedles filled with a vaccine agent; the microneedles comprising biodegradable polymers; and the microneedle assembly configured to release the vaccine agent at predetermined times with a predetermined amount of the vaccine agent while the microneedle assembly remains embedded in a subject. In one aspect, the biodegradable polymer degrades over time to release the therapeutic agent into the subject. In another aspect, the system further comprises a computer processor in electronic communication with the microneedle assembly, the computer processor programmed with computer readable instructions to initiate the release of the vaccine agent. In another aspect, the system further comprises an activator in electronic communication with the computer processor to initiate the release of the therapeutic agent. In another aspect, each of the microneedles in the plurality of microneedles is conical shaped. In another aspect, the vaccine agent is a protein or a nucleic acid. In another aspect, at least one of the microneedles in the plurality of microneedles includes a maximum height of about 600 μm.

Another embodiment is the use of the pulsatile vaccine delivery system described herein for the delivery of a vaccine to a subject in need thereof.

Another embodiment described herein vaccine delivery system comprising: an assembly comprising: (a) a substrate, and (b) a plurality of microneedles extending from the substrate, each microneedle comprising: a shell comprising a biodegradable polymer, a core comprising a vaccine agent, and a cap comprising a biodegradable polymer, the cap enclosing the core within the shell;

wherein the shell of each of the plurality of microneedles is selected to degrade ata predetermined time to release the vaccine agent therein into skin of a subject. In one aspect, the biodegradable polymer comprises poly(D,L-lactide-co-glycolide) (PLGA) or poly-lactide acid (PLA). In another aspect, the cap comprises PLGA or PLA. In another aspect, the shell of a first subset of the plurality of microneedles is selected to degrade at a first time to release the therapeutic agent therein into skin of the subject, and wherein the shell of a second subset of the plurality of microneedles is selected to degrade at a second time to release the therapeutic agent therein into the skin of the subject. In another aspect, the shell of a first subset of the plurality of microneedles is selected to degrade at a first time to release a first dose of the vaccine therein into skin of the subject, and wherein the shell of a second subset of the plurality of microneedles is selected to degrade at a second time to release a second dose of the vaccine therein into the skin of the subject. In another aspect, the core of at least one of the microneedles includes a maximum diameter of about 200 μm and a maximum height of about 300 μm. In another aspect, at least one of the microneedles in the plurality of microneedles includes a maximum height of about 600 μm.

Another embodiment is the use of the vaccine delivery system described herein for administering a vaccine to a subject in need thereof.

Another embodiment described herein is a method for administering a vaccine to a subject in need thereof, the method comprising: contacting a vaccine delivery system with skin of the subject, the vaccine delivery system comprising: an assembly comprising: a substrate, and a plurality of microneedles extending from the substrate, each microneedle comprising: a shell comprising a biodegradable polymer, a core comprising a vaccine agent, and a cap comprising a biodegradable polymer, the cap enclosing the core within the shell; wherein the shell of each of the plurality of microneedles is selected to degrade at a predetermined time to release the vaccine agent therein into the skin of the subject. In one aspect, the vaccine agent is a protein or a nucleic acid. In another aspect, the shell of a first subset of the plurality of microneedles is selected to degrade at a first time to release the therapeutic agent therein into the skin of the subject, and wherein the shell of a second subset of the plurality of microneedles is selected to degrade at a second time to release the therapeutic agent therein into the skin of the subject. In another aspect, the shell of a first subset of the plurality of microneedles is selected to degrade at a first time to release a first dose of the vaccine therein into the skin of the subject, and wherein the shell of a second subset of the plurality of microneedles is selected to degrade at a second time to release a second dose of the vaccine therein into the skin of the subject.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows an exemplary schematic of fully embedded microneedles (MNs) for stabilized mRNA vaccine delivery from a transdermal patch. FIG. 1B shows a graph of the typical multi-burst dermal release pattern of cumulative active mRNA from a single-administration MN patch. The single-administration patch contains two different sets of MNs that release mRNA at different programmable time points. FIG. 1C shows an exemplary illustration of MN contents that comprise a protamine-mRNA complex inside lipid nanoparticles (LNPs). The graphic showing the formation of the lipid-mRNA-protamine nanoparticles is adapted from the reference, Cellular immunology 354, 2020: 104143. FIG. 1D shows an exemplary MN patch having a unique core-shell for single-administration of vaccines. FIG. 1E shows a graph of MN loading capacity for solution vs. powder samples.

FIG. 2A-B show that stabilized mRNA (i.e., mRNA-protamine complex) contained inside lipid particles helps to enhance cell transfection. FIG. 2A shows a graph of luciferase luminescence assays demonstrating that the lipid-mRNA-protamine particles (with or without the addition of cryo-protectants) effectively transfect cultured cells, while control samples of naked mRNA or the complex of mRNA-protamine alone are not able to transfect the cells. The graphic showing the formation of the lipid-mRNA-protamine nanoparticles is adapted from the reference, Cellular immunology 354, 2020: 104143. Trehalose and sucrose were used for vacuum drying the mRNA-protamine complex. FIG. 2B shows an electrophoresis gel confirming the formation of a stable mRNA-protamine complex in citrate buffer. The first band at the bottom of the gel is background noise.

FIG. 3A-C show electrophoretic gel analyses of the heat stability of free mRNA (RNA) and mRNA-protamine complexes (RNA-Pro) with and without different sugar excipients of trehalose or sucrose. FIG. 3A shows a gel of RNA-Pro and RNA samples with no sugar. FIG. 3B shows a gel of RNA-Pro and RNA samples with different concentrations of trehalose. FIG. 3C shows a gel of RNA-Pro and RNA samples with different concentrations of sucrose.

FIG. 4A-C show data for the synthesized LNP-mRNA particles for expression of the luciferase protein reporter in cultured cells. FIG. 4A shows dynamic light scattering (DLS) characterization analysis to show the size distribution of the particles. FIG. 4B shows Zeta potential analysis to show that the particles are positively charged. FIG. 4C shows that LNP-mRNA transfection of cells induces bioluminescence from the luciferase expressed by the cultured cells compared to naked mRNA.

FIG. 5A-C show that the addition of cryo-protectants such as sucrose increases the stability of LNPs. FIG. 5A shows DLS data of LNP size without sucrose. FIG. 5B shows DLS data of LNP size with 20% sucrose. FIG. 5C shows the thermal stability (after 7 days of being exposed to 40° C.) of different formulations of LNP-mRNA with and without 20% sucrose to transfect cells.

FIG. 6 shows a proposed experimental flow chart for assessing in vivo immunogenicity of the stabilized mRNA via the use of simple dermal MNs in comparison with the use of multiple subcutaneous injections.

FIG. 7A-F show the fabrication and analysis of MNs for immediate release at time 0, using IgG as a vaccine model. FIG. 7A-B show schematics of the molding process to create the MNs which contain the vaccine inside a hydrophilic matrix of excipients (e.g., trehalose, sucrose, or PVP). FIG. 7C shows that the MNs are located on top of a supporting array of PLA coated with a highly water-soluble PVP layer to facilitate skin embedment. FIG. 7D shows an optical image of the vaccine loaded MNs. FIG. 7E shows optical images of rat skins receiving the MNs. The MNs can be easily embedded into the rat skin and the body fluid dissolves the PVP to leave behind arrays of the MNs which are dissolved inside the rat skin within a few hours. FIG. 7F shows a graph of the blood antibody titer (quantified by ELISA) showing the immediate immune response of the rats to the release of the IgG from the MNs in comparison with subcutaneous injections at time 0. The immune response to the MNs is even higher than that of the subcutaneous injection.

FIG. 8A-B show graphs of the release kinetics of the MNs carrying payloads of vaccine antigens in vitro and in vivo (i.e., rat skin). The burst releases are similar between the in vitro and in vivo conditions, demonstrating the reliability of the in vitro model (using PBS at 37° C.) to mimic the dissolution of the MNs in vivo.

FIG. 9 shows a graph of the cumulative release of mRNA from MNs having different Poly(D,L-lactide-co-glycolide) (PLGA) shells (PLGA1-8) with varying delayed release properties.

FIG. 10A-D show the effects of amino acids (e.g., 0.3% lysine) on mRNA formulation stability using bioluminescence in transfected HeLa cells at different time points and heat conditions. FIG. 10D shows bioluminescence data for formulations that were loaded into MN and exposed to different heat conditions.

FIG. 11 shows a graphic illustration of the thermally stabilized COVID-19 protein vaccine with built-in prime boosters.

FIG. 12A-D show graphs of the stabilization of the S1-RBD-protein antigen using Trehalose-Sucrose dry formulation at 100° C. for short-term screening over 60 minutes (FIG. 12A), 37° C. for long-term incubation over 3 months (FIG. 12B), 56° C. for short-term screening over 60 minutes (FIG. 12C), and 70° C. for short-term screening over 60 minutes (FIG. 12D) (n=3 independent experiments). Data are mean ±s.d.

FIG. 13 shows a Coomassie Blue-stained SDS-PAGE gel of S1-RBD under varying temperature conditions. Lane L—Ladder, Lane 1—Negative Control (DI Water), Lane 2—S1-RBD stock, Lane 3—S1-RBD after drying, Lane 4—S1-RBD after drying and exposed to 80° C. for 1 hr, Lane 5—S1-RBD+TS 1:4 v/v after drying, Lane 6—S1-RBD+TS 1:4 v/v after drying and exposed to 80° C. for 1 hr.

FIG. 14A-D show a new fabrication process with a high-throughput one-step MN core loading and testing of the S1-RBD protein loaded core-shell MNs. FIG. 14A shows assembly process illustrations of the MN patch. (i-ii) PLGA polymer is compression molded into a negative PDMS MN mold under heating. (iii-iv) Arrays of micro-molded prepared vaccine cores are loaded into arrays of MNs shell via a one-step alignment process. (v) After removing the scum layer, a supporting array of PLA-effervescent with the cap-layer is aligned and assembled with the core-shell MNs through a heat-sintering process. (vi) The PDMS mold is peeled off to obtain the free-standing core-shell MNs on the supporting array. FIG. 14B shows an optical image of the MN patch containing S1-RBD protein (scale bar=1 cm). FIG. 14C shows an optical image of fabricated core-shell MNs (scale bar=300 μm). FIG. 14D shows the mechanical behavior of S1-RBD loaded core-shell MNs made of PLGA 50:50 30 kDa under compressive force. The dashed line represents insertion force to human skin of average 0.058 N/needle. Data are mean ±s.e.m.; n=3 different MN patches.

FIG. 15 shows a graph of the cumulative in vitro release of a core-shell MN patch loaded with Rhodamine B fluorescence dye with different molecular weight PLGA shells. n=3 independent experiments. Core-shell MN patches (about 200 needles/patch) loaded with sulforhodamine B dye were fabricated from different PLGA shells. The dye was used as a model for studying release kinetics. Each patch was incubated in 2 mL of PBS (pH 7.4) in a shaking water bath at 37° C. The supernatants were collected every 24 hr and replaced with fresh buffer. Samples were analyzed using a fluorescence microplate reader (BioTek) at 565/586 nm.

FIG. 16 shows graphs of a dose study on the antibody response of rats receiving multiple subcutaneous injections of S1-RBD protein at 10 μg/dose, 20 μg/dose, 50 μg/dose, and 100 μg/dose and analyzed by ELISA. Serum was diluted 1:10 in 0.05% Tween-20 in PBS. Bars are mean ±s.e.m.; n=4-5 rats per group. Statistical analysis was performed using two-way analysis of variance (ANOVA) with Repeated Measures with Dunn's multiple comparisons test.

FIG. 17 shows a graph of dose matching between an MN patch and a subcutaneous bolus injection. Bars represent mean ±s.d. (n=3 independent experiments). After S1-RBD MN core fabrication, the patches were dissolved in PBS buffer and assessed by ELISA.

FIG. 18A-F show the immunogenicity of the S1-RBD protein-loaded core-shell MN patch. FIG. 18A shows the vaccination and serum collection schedule. FIG. 18B shows erythema and eschar formation, and Edema formation on the rat skins. Data are mean ±s.e.m. n=5 rats per group, independent experiments. Scores range from 0 (normal) to 4 (severe). FIG. 18C shows the immune responses over the course of time from the rats receiving the S1-RBD protein loaded core-shell MNs, multiple bolus subcutaneous injections, and empty MNs. Bars represent mean ±S.E.M. (n=4-5 rats per each group, independent experiments). FIG. 18D shows end-point IgG titers of serum collected on Day 70 that were analyzed by ELISA. Statistical analysis was determined via One-way ANOVA with Tukey's multiple comparisons post-hoc test. FIG. 18E-F show plaque reduction neutralization test (PRNT) results of serum collected on Day 70 against US-WA1/2020 and Delta B.1.617.2 variants. Bars represent mean ±s.e.m. (n=4-5 rats per each group, independent experiments). Statistical analysis was determined via Kruskal-Wallis ANOVA with Dunn's multiple comparisons test.

FIG. 19 shows graphs of the pH stability of S1-RBD protein dissolved in degrading PLGA solution. As PLGA films of different molecular weights that were incubated at 37° C. in PBS (pH 7.4) degraded, they produced acidic byproducts which lowered the solution pH. The stability of S1-RBD protein was assessed using ELISA. Bars represent mean ±s.d. (n=3 independent experiments).

FIG. 20 shows optical images of rats' skin, taken throughout the S1-RBD protein vaccination study for skin-irritation assessment. No significant irritation was observed from the long-term implantation of the S1-RBD loaded core-shell MNs (n=5, independent experiments).

FIG. 21A-E show representative histology staining of rats' skin tissues after 70 days following MN implantation (FIG. 21A, 21C, and 21D), or following multiple subcutaneous injections (FIG. 21B and 21E). Images viewed at 4× magnification of Hematoxylin and eosin staining. Scale bar indicates 100 μm. n=4-5 rats, independent experiments.

FIG. 22 shows the immunogenicity of the S1-RBD protein-loaded core-shell MN patch compared with subcutaneous injections of the same protein antigens over the course of vaccination schedule. Serum was diluted 1:10 in 0.05% Tween-20 in PBS. Statistical analysis was determined via One-way ANOVA with Tukey's multiple comparisons post-hoc test. Bars represent mean ±s.e.m. (n=4-5 rats per each group, independent experiments).

FIG. 23A-C show graphic illustrations of the single-administration long-acting antibody-delivery microarrays (MA) with an ultrahigh cargo-loading capacity for multiple burst releases of thermally-stabilized antibodies. FIG. 23A shows a schematic describing the concept of core-shell MA, with ultrahigh cargo-loading capacity and pre-programmed longitudinal doses of thermally stabilized mAb via a single-administration, to sustain mAb plasma concentration. The MA could also co-deliver different antibodies into circulation at the same time. FIG. 23B shows the new powder-filling technique for high-dose biomolecules loaded into the MA patches. (i) Dry powders are weighed and filled onto the empty PDMS mold in small parts. The PDMS mold is then compressed under vacuum. These processes are repeated until all powders are filled in. (ii) Excipients or polymer solutions are casted onto PDMS mold. (iii) The MA patch solidifies after a drying process. FIG. 23C shows a traditional solution-casting technique fabrication process. (i) Dry powders are solubilized or dispersed into excipients or polymer solutions at suitable concentrations. (ii) The solution is then casted onto empty PDMS mold. (iii) The MA patch solidifies after a drying process.

FIG. 24A-C show the ultrahigh antibody-loading capacity of the MA patches from the new powder-loading method. FIG. 24A shows a comparison between loading efficacy of human IgG (hIgG) in a 1×1 cm² MA patch fabricated by the two different techniques (the new powder loading and traditional solution casting methods). Data are mean ±s.d. n=3, independent experiments. FIG. 24B shows an image of a large MA patch containing multiple 1×1 cm² patches. The 1×1 cm² patches are considered “pixels” and can be assembled with as many as desired (to increase the total antibody dose) into a single patch for a single-time skin administration. FIG. 24C shows a zoomed-in image of the MNs in the MA patches containing the hIgG antibodies fabricated with the powder-filling technique.

FIG. 25A-C show preliminary screenings of excipient formulations to thermally stabilize rat IgG antibody. The antibody was formulated in different excipients formulations, all at a concentration of 20 μg/ml. The excipients included Polyvinylpyrrolidone (PVP), Sorbitol, Trehalose, Sucrose, and Trehalose+Sucrose (TS). The formulations were left vacuum dried overnight (mimicking the MA fabrication process). Short-term exposures to high heat at (FIG. 25A) 56° C., (FIG. 25B) 70° C., and (FIG. 25C) 100° C. for 1 hr were performed. The stability of rIgG antibody was measured using ELISA and normalized to stock antibodies. Bars represent mean ±s.d. (n=3, independent experiments).

FIG. 26A-E show the thermal stabilization of antibodies using Trehalose-Sucrose vacuum-dry formulations. FIG. 26A shows short-term thermal stability of hIgG with and without excipients at an extreme temperature of 100° C. for 1 hr. FIG. 26B shows long-term thermal stability of hIgG with and without excipients at 37° C. for up to 90 days. The stability of hIgG antibody was measured using ELISA and normalized to the stock antibody. Bars represent mean ±s.d. (n=3, independent experiments). FIG. 26C shows a Coomassie Blue-stained SDS-PAGE gel of hIgG exposed to 100° C. for 1 hr to show the sustained overall structure of the antibody. FIG. 26D shows a Coomassie Blue-stained SDS-PAGE gel of hIgG exposed to 37° C. for 90 days. FIG. 26E shows the effect of MA manufacturing and heat exposure on the anti-HIV activity of the b12 antibody. HIV-1 BaL virus, b12 (ng/mL) antibody, and TZMbI cells were co-incubated for 48 hr. Data represent means ±SD of N=4 experiments (each concentration was run in triplicate).

FIG. 27A-B show stabilization of antibodies using Trehalose-Sucrose dry formulations. Short-term thermal stability of hIgG was tested with and without excipients at (FIG. 27A) 56° C. for 1 hr and (FIG. 27B) 70° C. for 1 hr. Data are mean ±s.d. N=3, independent experiments. The stability of rIgG antibody was measured using ELISA and normalized to stock antibodies.

FIG. 28A-C show the pharmacokinetic profiles of high dose human Ig antibodies delivered using the MA patch. FIG. 28A shows a dose-dependent study on the PKs when MA patches with different dosages of hIgG were administered to rats. FIG. 28B shows co-delivery of multiple human Ig (human IgG1, IgG2, and IgA) via a single-time administration of the MA patches. FIG. 28C shows sustained delivery of hIgG using single-administration core-shell MA patches with built-in multiple delayed burst releases. Data are mean ±s.e.m. N=4-5 rats per group, independent experiments.

FIG. 29A-B show skin evaluation of high-dose antibody-loaded MA patch insertion. FIG. 29A shows erythema and eschar formation and FIG. 29B shows edema formation on the rat skins. Data are mean ±s.e.m. n=4-5 rats per group, independent experiments. Scores range from 0 (normal) to 4 (severe).

FIG. 30A-C show a thermal stability assessment of human (FIG. 30A) IgG1, (FIG. 30B) IgG2, and (FIG. 30C) IgA antibodies. The MA patches loaded with different antibodies with or without Trehalose-Sucrose (TS) excipients were fabricated and incubated at 70° C. and 100° C. for 1 hr. The stabilities of antibodies were measured using ELISA and normalized to stock antibodies. Bars represent mean ±s.d. (n=3, independent experiments).

FIG. 31 shows the pH stability of human IgG antibody dissolved in degrading PLGA solution (low pH environment). PLGA films of different molecular weight PLGA 50:50 30 kDa and PLGA 75:25 80 kDa were incubated at 37° C. in PBS (pH 7.4). As the films degraded, they produced acidic byproducts which lowered the solution pH. This is to mimic the condition of MA shell degradation. Stock of hIgG was mixed with degraded PLGA solution at 1 mg/mL. The stability of hIgG was assessed using ELISA. Bars represent mean ±s.d. (n=3, independent experiments).

FIG. 32 shows the anti-human IgG antibody response in rats after receiving one dose of human IgG via immediate release MA at Day 0. Following MA loaded hIgG administrations, the rats develop antibody responses against hIgG. Bars represent mean ±s.e.m. (n=3, independent experiments). The rats received one dose of 0.14 mg hIgG loaded immediate release MA patch at Day 0. Serum samples from rats were collected and assessed for anti-human IgG antibody response using ELISA and compared with rats receiving subcutaneous injections of sterile saline.

FIG. 33 shows in vivo release studies of human IgG from the MA patches. One group of rats received 2 mg stabilized hIgG loaded immediate release MA patch (Day 0 release). Another group of rats received 2 mg stabilized hIgG loaded core-shell MA patch (Day 10 release). Serum samples from rats were collected and analyzed for plasma concentrations of hIgG using ELISA. Bars represent mean ±s.e.m. (n=3, independent experiments).

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not corn pletely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the term “protamine” refers to positively charged polypeptides. Protamines are small, arginine-rich, nuclear proteins that replace histones late in the haploid phase of spermatogenesis and are believed essential for sperm head condensation and DNA stabilization. In humans, there are two forms of protamine, Protamine 1 and Protamine 2, as shown:

| | | |
|---|---|---|
| Protamine 1 | MARYRCCRSQSRSRYYRORQRSRRRRRRSCQTRRRAMRCCRPRYRPRCR | SEQ ID NO: 1 |
| | R | |
| Protamine 2 | MVRYRVRSLSERSHEVYRQQLHGQEQGHHGQEEQGLSPEHVEVYERTHG | SEQ ID NO: 2 |
| | QSHYRRRHCSRRRLHRIHRRQHRSCRRRKRRSCRHRRRHRRGCRTRKRT | |
| | CR | |

Other protamines, such as from mouse, rat, rabbit, or other mammals may also be used.

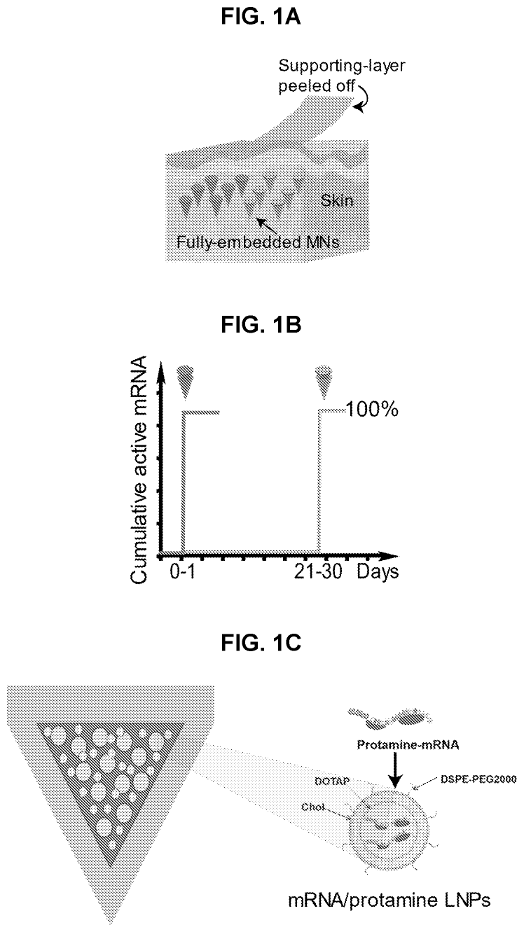

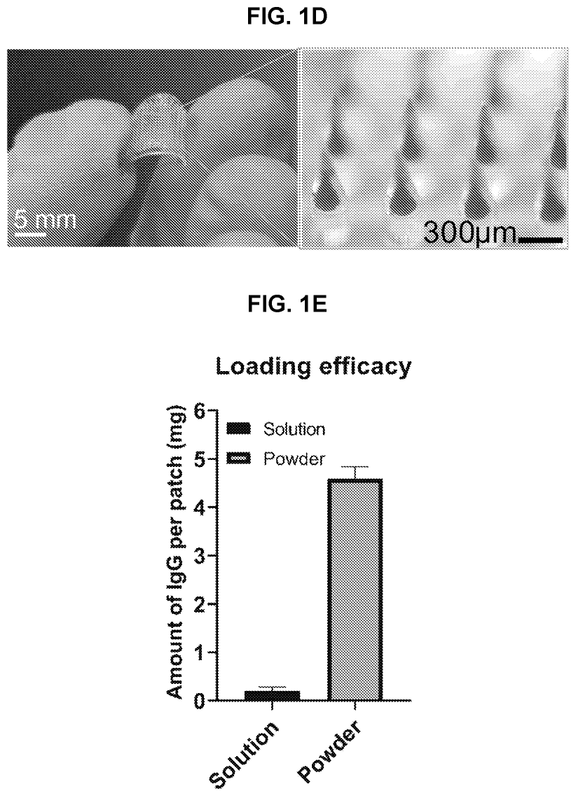

Described herein are compositions and methods directed to the thermal stabilization of RNA and protein antigens for long-term storage and use in transdermal microneedle (MN) patches. The present disclosure describes the preparation of a dry formulation of mRNA complexed with protamine that can also be mixed with lipid nanoparticles (LNPs) to enhance the cell transfection efficiency of the mRNA (FIG. 1C). Moreover, the formulations disclosed herein may be employed within a transdermal MN patch for vaccination applications, which is easily fabricated with the dry formulation of the mRNA to highly transfect cells at the transdermal layer, rather than subcutaneous or intramuscular delivery as in conventional vaccination practice.

A drug delivery system described herein provides a one-time administration of one or more therapeutic agents of one or more dosages which are programmed to release at predictable time points. For example, degradation of the polymeric shell of the microneedles provides for release of the mRNA or protein therapeutic or vaccine agents. The drug delivery system can replace multiple bolus injections of many therapeutic agents, especially vaccines which require initial dose and multiple boosters.

The drug delivery system includes core shell microneedles that provide a minimally invasive approach and allow for self-administration. Importantly, for vaccine delivery, the needles pierce into the dermis layer of the skin where there is a substantial number of immune cells, thereby significantly enhancing immune response. Microneedles are, therefore, an appealing delivery approach to enable one-time, painless, and effective administration of vaccines to replace multiple injections in the conventional immunization process. The microneedles are fabricated from FDA approved materials (e.g., poly(D,L-lactide-co-glycolide) (PLGA), poly-lactide acid (PLA)) that are commonly used for drug delivery, medical devices, etc.

The master structures for the microneedles are fabricated using a 3D laser lithography system as described in U.S. Pat. Pub. No. US 2019/0269895 A1, which is incorporated herein by reference in its entirety. Inverse molds comprising silicone (polydimethylsiloxane (“PDMS”)) are made by pouring PDMS solution over the master structure and curing at 60° C. for 3 hours. A process is employed which only relies on common PDMS molds to eliminate the scum layer. The fabrication process comprises the steps of pressing a needle mold of PDMS onto a hot Poly(D,L-lactide-co-glycolide) (“PLGA”). The PLGA is trapped inside the cavity of the mold, which is then separated from the substrate. The released mold has a significant scum layer which needs to be removed to create free-standing and fully-embeddable needles. To achieve this, the PDMS mold (using spin-coater) is spun and solvent (e.g., acetone, dimethyl sulfoxide (“DMSO”)) or water (to remove hydrophilic drug) is gently dropped on top to remove the scum layer. This spinning process can be repeated until the scum layer is completely removed.

The present disclosure provides an advantageous system and method directed to a method to stabilize mRNA for (1) long-term use against thermal instability and (2) the use in transdermal MN patches which can be applied into skin either for immediate release of the mRNA or delayed release of the mRNA to create single-administration mRNA delivery microneedle patch with built-in booster release bursts.

The system, composition, method described herein may be utilized to produce mRNA-and protein-based vaccines, therapeutic drugs for cancer, infectious diseases, diabetes, heart diseases, and variations thereof.

The method disclosed herein is unique because it combines the protamine with LNPs and a drying process with stabilizing excipients to (1) formulate the mRNA with protamine to form the complex mRNA-protamine and stabilize the mRNA, (2) encapsulate the complex inside a lipid entity (using liposome or lipid nanoparticles) to enhance cell transfection of the mRNA and further stabilize it, and (3) dry the mRNA formulations inside a mixture of excipients (e.g., trehalose, sucrose, mannitol, silk, or a combination thereof), which will enable a long-term thermal stability of the mRNA. The present disclosure describes the development and characterization of a single-administration MN patch for both stabilized RNA and protein vaccine delivery. These MNs only require a single-time and painless skin application (similar to a nicotine patch) while repeatedly delivering stabilized mRNA vaccines into the skin at programmable time points, similar to multiple bolus prime booster injections (FIG. 1A-B). With these single-administration MN patches, patients will not be required to remember a specific vaccine schedule and will have no concern on how to store the vaccine patches appropriately, which is required for conventional MN patches such as Nanopatch™ for long-time repeated uses. The MNs have a core-shell structure in which a vaccine core is encapsulated by a biodegradable polymeric shell. By tuning the shell degradation time, the burst release time of the encapsulated vaccines can be programmed from few hours to several days and months. Excipient formulations have been developed to stabilize a variety of protein-based vaccine models (e.g., OVA, Prevnar13™, S-protein of the Covid-19 virus, etc.) and single-administration MNs have been shown to successfully deliver the vaccines with the same immunogenic effect as that obtained from multiple injections. Various MN forms may be used for the disclosed invention.

mRNA is well known to be unstable, especially to heat and other RNAs inside the body. Thus, the use of this genetic material requires a process to chemically modify or complex the mRNA with LNPs or other positive cationic polymers for stabilization. So far, the use of LNPs has shown significant advances in stabilizing mRNA from enzymatic attack. Furthermore, the lipid capsule enhances the cellular uptake and facilitates endosomal escape of the mRNA for the translation of encoded proteins inside host cells. Although the LNPs or liposome particles are prone to aggregation and are unstable under a high heat application, recent works have shown that the use of cryo-protectant agents such as sucrose and trehalose can significantly enhance the stability of the LNPs. Unfortunately, the use of LNPs seems to not be enough to protect mRNA from heat. Consequently, the mRNA LNPs still need to be kept at a stringent cold temperature which limits its application for places or countries with poor conditions of vaccine cold-chain storage.

Recently, it has been found that the cationic protein protamine can electrostatically bind mRNA and stabilize the mRNA at high temperatures (70° C. and 37° C.) for a long incubation period (up to several months). In terms of mechanism, the protamine binds to the negatively-charged mRNA to prevent this genetic material from being folded or flipped around, which allows the hydroxyl group (—OH) to interact with phosphodiester bonds and further self-hydrolyze them to break down the mRNA. The complex of mRNA and protamine can even be encapsulated inside the lipid particles for MN loading (FIG. 1C), which can potentially boost the stability of the mRNA while still offering the excellent ability to transfect cells and express proteins of interest.

Different composition components of the present disclosure that can be used to stabilize mRNA include but are not limited to: (1) cationic peptides and polypeptides, including protamine, poly-lysine, poly-arginine, poly-histidine, and other cell penetrating peptides; (2) other cationic and zwitterionic compounds, including cationic and zwitterionic lipids and lipid-like materials (e.g., dioleoyl-3-trimethylammonium propane (DOTAP), DDAB, DOTMA, dioleoylphosphatidylethanolamine (DOPE), DC-cholesterol, DOSPA, DOGS, DSPC, DLin-MC3-DMA) and their formulated liposomes, cationic dendrimers (e.g., PAMAM), cationic polymers (e.g., polyethylenimine (PEI), poly(dimethylaminoethyl methacrylate) (PDMAEMA), poly-β-amino esters (PAE), (4-aminobutyl)-L-glycolic acid (PAGA), polymers (polyester, polycarbonate, polypeptide, poly(metha)acrylate, poly(allyl ethylene phosphate) that are modified to contain amino groups (primary, secondary, tertiary, and quaternary), guanidino groups and imidazole groups), cationic saccharides (e.g., chitosan, cationic (cyclo) dextrin, cationic cellulose and cationic dextran), cationic proteins (e.g., avidin, gelatin, collagen, elastin and elastin like polymer (ELP), albumin); zwitterionic polymers, zwitterionic saccharides, zwitterionic polypeptides and zwitterionic proteins containing aforementioned cationic components; negatively charged polymers, saccharides, (poly)peptides and proteins but with a cationic center, a cationic pocket and a cationic portion which consists of the aforementioned cationic components; and (3) cryoprotectants, including sugars (e.g., sucrose, mannitol, trehalose), polyols, and amino acids (e.g., arginine, lysine, histidine). These different components can be added and mixed together in any order with an mRNA of interest to optimize stability.

In one nonlimiting exemplary embodiment, a MN which only contains the stabilized mRNA (i.e., mRNA complexed with protamine) and having immediate release properties is described. Using this MN form, patients would likely need to repeatedly apply the MNs into their skin at different time points (e.g., day 0 and day 21). In another nonlimiting exemplary embodiment, a single-administration MN may be used.

For example, a core-shell MN may be used as described in U.S. Pat. Pub. No. US 2019/0269895 A1, which is incorporated herein by reference in its entirety. By applying these core-shell MNs into the skin at time 0, patients will automatically receive the booster release bursts at desired time points without needing to repeatedly apply or store the MN patches.

In another nonlimiting exemplary embodiment, to develop formulations for stabilizing mRNA, mRNA is first used to encode the luciferase protein as an mRNA model for the study of thermal stability. When added with luciferin, the luciferase protein emits bioluminescence and the transfection efficiency and subsequent mRNA bioactivity can be tested by performing an in vitro assay on cultured cells (including dendritic cells, muscle cells, and adipose stem cells which are common cells in the skin). Stabilizing excipients of sucrose and trehalose, which are known cryo-protectants, will be used to stabilize LNPs. For the mRNA, cationic protamine will be employed, which can bind to mRNA via the electrostatic force to prevent the molecule from flipping around and being denatured by self-hydrolysis.

Also described herein is a new protein-stabilization method and high-throughput antigen-loading process to create a single-administration MN skin patch which could deliver multiple doses of a recombinant S1-RBD protein vaccine against SARS-CoV-2 viruses. The single-administration microneedle platform addresses the COVID-19 crisis and could be used for potential future outbreaks.

Transdermal microneedle (MN) skin patches offer an excellent candidate for such a delivery system. MNs can be self-administered by patients at homes and may not require a skilled healthcare professional. In contrast to conventional bolus injections by hypodermic needle, the MNs are administered more superficially, avoid touching the nerve ends and are, thus, almost painless. Furthermore, the MNs target the dermal layer wherein there are many immune cells including dendritic Langerhans cells which help to boost the immune reaction to vaccines.

Although new mRNA or vector-based COVID-19 vaccines can be rapidly produced and have been successfully implemented, traditional vaccines using recombinant proteins have their own merits in the global vaccination effort due to their proven long-term safety profile, the widespread availability of manufacturing facilities, and lower manufacturing costs. Indeed, at this moment, many vaccines using the spike protein (S-protein) antigens (spike glycoprotein projecting externally from the shell of SARS-CoV-2 virus) have emerged for clinical use and even shown to be effective against different COVID-19 strains including the highly contagious Delta variant (e.g., FDA-approved Novavax™ with >90% efficacy against symptomatic SARS-CoV-2 virus as of December 2021). Among those S-protein based antigens, the receptor binding domain (RBD) subunit could trigger broad and durable neutralizing antibodies against the SARS-CoV-2. Moreover, studies on MN patches with instantaneous release of the S-protein have shown exciting success, demonstrating the potential benefits of the application of the technology for COVID-19 vaccine delivery.

Despite providing substantial immune protection, most available vaccine MN patches so far have been designed to only perform an immediate delivery of vaccine antigens, which thus requires repeated administrations for boosters. Patients are still responsible for keeping up with the vaccination schedule and need to store the MN patches appropriately (to avoid thermal degradation) for the follow-up booster administrations. Some researchers have developed MNs for the sustained delivery of small-molecule drugs or vaccines, however, there is a risk that the sustained release of vaccines could elicit immune tolerance responses. These shortcomings of current MN patches together make them suboptimal for use in global vaccination efforts.

In this context, the use of single-administration multi-burst release core-shell MNs for the delivery of a clinically relevant vaccine against Streptococcus pneumoniae Prevnar13™ has recently been reported. However, Prevnar13™ is quite stable and can maintain structural integrity/immunogenicity over the course of the MN fabrication process and skin-implantation. In contrast, many other important vaccines are highly susceptible to damage or denaturation by heat. Furthermore, the previously reported fabrication approach relies on a multiple-step process to load vaccine cores into the MNs, which reduces the overall throughput of the manufacture and potentially causes loss of the loaded vaccine. Thus, there remain significant challenges to (1) create such single-administration MNs (often undergoing a high temperature fabrication process of 45-75° C.) for other thermally-unstable antigens such as the one against SARS-CoV-2, (2) store the MNs for a long-term use at a normal room temperature without the cold-chain condition, (3) enhance the throughput for the process to load vaccine-antigen into the MNs, and (4) embed the MNs inside the skin (at 37° C.) for a long period of time to perform the desired multiple burst release of the stabilized antigens, and trigger a long-term high quality antibody similar to the effect of multiple primer-booster injections.

Disclosed herein is a new strategy to thermally stabilize protein antigens (using the S1-RBD of SARS-CoV-2 as a model) and devise a one-step antigen-loading process to fabricate easy-to-use, painless and single-administration MNs which can be fully inserted into the skin and programmed to release the stabilized vaccine at multiple desired times. The present disclosure describes how the S1-RBD protein antigen can be stabilized at an extreme temperature, up to 100° C., for a short screening time (at least 1 hour) and at the body temperature (37° C.) for at least 4 months. The stabilized antigen can then be loaded into the core-shell MNs via a single-alignment step. The MNs are inserted into the skin of rats in a minimally invasive manner and exhibit no noticeable skin irritation even after a long-term implantation. These MNs successfully trigger antibody responses which are capable of neutralizing authentic SARS-CoV-2 strains, including the Delta variant, at similar levels to those induced by multiple bolus injections of the same vaccine antigens stored in conventional cold-chain conditions. The antigen-stabilization approach and the antigen-loading method for fabricating the single-administration MN platform, presented herein, could provide the key solution to the global vaccination campaign (against the current/future pandemics) by avoiding (1) the pain, cost, and low patient compliance/adherence to the conventional vaccine injections, (2) the requirement of cold-chain facilities, and (3) the need of multiple booster shots.

One embodiment described herein is a stabilized RNA vaccine composition, the composition comprising: a complex of RNA with one or more cationic polymers; and one or more cationic lipid entities. In one aspect, the RNA comprises mRNA, miRNA, siRNA, tRNA, rRNA, or other RNA. In another aspect, the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof. In another aspect, the cationic polymer comprises protamine. In another aspect, the one or more cationic lipid entities comprises liposomes or lipid nanoparticles. In another aspect, the liposomes or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof. In another aspect, the composition further comprises one or more cryoprotectants. In another aspect, the one or more cryoprotectants comprises one or more of trehalose, mannitol, sucrose, glucose, fructose, lactose, dextran, glycerol, ethylene glycol, propylene glycol, polyethylene glycol, or combinations thereof. In another aspect, the composition further comprises one or more amino acids selected from lysine, arginine, histidine, glutamine, proline, glycine, threonine, or combinations thereof. In another aspect, the complex of RNA with one or more cationic peptides has a size ranging from about 20 nm to about 500 nm. In another aspect, the cationic lipid entity has a size ranging from about 20 nm to about 500 nm. In another aspect, the stabilized RNA is thermally stable for at least about 2 months at about 37° C. In another aspect, the stabilized RNA is thermally stable for at least about 2 hours at about 80° C.

Another embodiment described herein is method for stabilizing RNA, the method comprising: (a) forming a complex comprising the RNA with one or more cationic polymers; (b) mixing the complex with one or more cationic lipid entities comprising liposomes or lipid nanoparticles to form a lipid mixture; and (c) drying the lipid mixture under vacuum. In one aspect, the one or more cationic polymers comprise one or more of protamine, poly-lysine, poly-arginine, poly-histidine, amino group modified polycarbonate, amino group modified polypeptide, -poly-β-amino esters, cationic cellulose, cationic dextran, cationic cyclodextrin, or combinations thereof. In another aspect, the cationic polymer comprises protamine. In another aspect, the liposome or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoyl phosphatidylethanolamine (DOPE), or combinations thereof. In another aspect, the method further comprises mixing the lipid mixture with one or more cryoprotectants comprising one or more of, trehalose, mannitol, sucrose, glucose, fructose, lactose, dextran, glycerol, ethylene glycol, propylene glycol, polyethylene glycol, or combinations thereof prior to drying the lipid mixture. In another aspect, the method further comprises mixing the lipid mixture with one or more amino acids selected from lysine, arginine, histidine, glutamine, proline, glycine, threonine, or combinations thereof prior to drying the lipid mixture.

Another embodiment described herein is a stabilized RNA vaccine prepared by the methods described herein.

Another embodiment described herein is the use of a stabilized RNA prepared by the methods described herein in the preparation of a vaccine medicament.

Another embodiment described herein is a stabilized vaccine agent composition, the composition comprising: a vaccine agent; and one or more cryoprotectants comprising one or more of trehalose, mannitol, sucrose, glucose, fructose, lactose, dextran, glycerol, ethylene glycol, propylene glycol, polyethylene glycol, or combinations. In one aspect, the one or more cryoprotectants have a concentration of about 0.3 M to about 1 M. In another aspect, the vaccine agent and the one or more cryoprotectants are present in a ratio ranging from about 1:1 (v/v) to about 1:6 (v/v). In another aspect, the vaccine agent is a protein, functional fragment thereof, or combination thereof. In another aspect, the composition is dried by lyophilization, vacuum, or dessication. In another aspect, the vaccine agent is thermally stable for at least about 4 months at about 37° C. In another aspect, the vaccine agent is thermally stable for at least about 1 hour at about 100° C.

Another embodiment described herein is a method for preparing a stabilized vaccine agent core, the method comprising: (a) mixing a vaccine agent with one or more cryoprotectants comprising one or more of trehalose, mannitol, sucrose, glucose, fructose, lactose, dextran, glycerol, ethylene glycol, propylene glycol, polyethylene glycol, or combinations thereof; and (b) drying the mixture. In one aspect, the one or more cryoprotectants have a concentration of about 0.3 M to about 1 M prior to drying. In one aspect, the vaccine agent is mixed with the one or more cryoprotectants at a ratio ranging from about 1:1 (v/v) to about 1:6 (v/v) prior to drying. In another aspect, the vaccine agent is a protein, functional fragment thereof, or combination thereof. In another aspect, drying in step (b) is performed by lyophilization, vacuum, or dessication. In another aspect, drying in step (b) is performed for at least about 4 hours to about 24 hours.

Another embodiment described herein is a stabilized vaccine core prepared by the methods described herein.

Another embodiment described herein is the use of a stabilized vaccine core prepared by the methods described herein the preparation of a vaccine medicament.

Another embodiment described herein is a method for one-step loading a vaccine core into a microneedle, the method comprising: (a) generating a core-shell microneedle assembly comprising a polymeric shell by compression-molding a film of poly(D,L-lactide-co-glycolide) (PLGA) into a silicone or poly(dimethylsiloxane) (PDMS) mold to form an assembly of microneedle shells, wherein the mold has an array of conical shaped cavities; (b) generating a vaccine core array by combining a vaccine agent with a cryoprotectant and casting the vaccine core on a second poly(dimethylsiloxane) (PDMS) mold having the same array of conical shaped cavities as the microneedle mold and drying the vaccine core; (c) transferring the vaccine core array onto a PLGA film; (d) aligning the vaccine core array on the PLGA film with the microneedle assembly, thereby filling the cores in the microneedle shell assembly with the vaccine cores; and (e) curing the microneedle assembly by vacuum heating to encapsulate the vaccine cores within the microneedles. In one aspect, generating a core-shell microneedle structure further comprises: removing an excess PLGA scum layer. In another aspect, the microneedles have a height of about 600 μm and a base diameter of about 300 μm. In another aspect, the vaccine cores have a height of about 400 μm and a diameter of about 200 μm.

Another embodiment described herein is a one-step loaded microneedle manufactured by the methods described herein.

Another embodiment described herein is the use of a one-step loaded microneedle manufactured by the methods described herein to deliver a vaccine to a subject in need thereof.

Another embodiment described herein is a method for powder-filling a microneedle assembly, the method comprising: (a) preparing a poly(dimethylsiloxane) (PDMS) mold having an array of conical shaped cavities on a silicon wafer; (b) dispersing a powdered vaccine agent into the mold cavities; (c) compressing the powdered vaccine agent into the mold cavities; (d) repeating steps (b) and (c) until the microneedle cavities are filled; (e) casting a layer of one or more sugars and water-soluble polymer over the filled cavities to generate a microneedle assembly; and (f) drying the microneedle assembly. In one aspect, steps (b)-(d) are performed in a neutralized static environment. In another aspect, the vaccine agent loading capacity is about 20-fold greater than solution-casting core filling. In another aspect, the vaccine agent is a protein, a nucleic acid, or a combination thereof.

Another embodiment described herein is a filled microneedle assembly manufactured by the methods described herein.

Another embodiment described herein is use of a filled microneedle assembly manufactured by the methods described herein to deliver a vaccine to a subject in need thereof.

Another embodiment described herein is a method for manufacturing a microneedle assembly, the method comprising: (a) generating a core microneedle assembly by: filling a first silicone mold including a plurality of microneedle cavities with a copolymer, spinning the first silicone mold to remove a first scum layer of the copolymer on the first silicone mold, and generating a core in each of the copolymer-filled cavities; (b) generating a therapeutic agent assembly by: filling a plurality of cavities in a second silicone mold with a therapeutic agent, spinning the second silicone mold to remove a second scum layer of the therapeutic agent, and removing the molded therapeutic agent from the second mold and transferring the molded therapeutic agent to a substrate; and (c) aligning the therapeutic agent assembly with the core microneedle assembly to thereby fill the cores in the core microneedle assembly with the therapeutic agent. In one aspect, the cavities in the first silicone mold and the second silicone mold are conical shaped. In another aspect, the copolymer is poly(D,L-lactide-co-glycolide) (PLGA). In another aspect, the method further comprises generating the first silicone mold by pouring polydimethylsiloxane (PDMS) over a master structure and curing. In another aspect, the vaccine agent is a protein or a nucleic acid. In another aspect, the method further comprises generating a cap assembly by: (a) filling a plurality of cavities in a third silicone mold with a copolymer to form a plurality of caps; (b) spinning the third silicone mold to remove a third scum layer of the copolymer on the third silicone mold; (c) removing the molded caps from the third mold and transferring the molded caps onto the core microneedle assembly; and (d) aligning the cap assembly with the core microneedle assembly to thereby cover the cores in the core microneedle assembly with the caps. In another aspect, the method further comprises applying heat to bond the cap assembly to the core microneedle assembly. In another aspect, the method further comprises coating a supporting array with a water-soluble polymer and contacting the supporting array with the cap assembly. In another aspect, the method further comprises applying heat to bond the supporting array to the cap assembly for removing the microneedle assembly from the first silicone mold.

Another embodiment described herein is microneedle device, manufactured by the methods described herein.

Another embodiment described herein is the use of a microneedle device, manufactured by the methods described herein for the delivery of a vaccine to a subject in need thereof.

Another embodiment described herein is a pulsatile vaccine delivery system comprising: a microneedle assembly including a plurality of microneedles filled with a vaccine agent; the microneedles comprising biodegradable polymers; and the microneedle assembly configured to release the vaccine agent at predetermined times with a predetermined amount of the vaccine agent while the microneedle assembly remains embedded in a subject. In one aspect, the biodegradable polymer degrades over time to release the therapeutic agent into the subject. In another aspect, the system further comprises a computer processor in electronic communication with the microneedle assembly, the computer processor programmed with computer readable instructions to initiate the release of the vaccine agent. In another aspect, the system further comprises an activator in electronic communication with the computer processor to initiate the release of the therapeutic agent. In another aspect, each of the microneedles in the plurality of microneedles is conical shaped. In another aspect, the vaccine agent is a protein or a nucleic acid. In another aspect, at least one of the microneedles in the plurality of microneedles includes a maximum height of about 600 μm.

Another embodiment is the use of the pulsatile vaccine delivery system described herein for the delivery of a vaccine to a subject in need thereof.

Another embodiment described herein vaccine delivery system comprising: an assembly comprising: (a) a substrate, and (b) a plurality of microneedles extending from the substrate, each microneedle comprising: a shell comprising a biodegradable polymer, a core comprising a vaccine agent, and a cap comprising a biodegradable polymer, the cap enclosing the core within the shell; wherein the shell of each of the plurality of microneedles is selected to degrade ata predetermined time to release the vaccine agent therein into skin of a subject. In one aspect, the biodegradable polymer comprises poly(D,L-lactide-co-glycolide) (PLGA) or poly-lactide acid (PLA). In another aspect, the cap comprises PLGA or PLA. In another aspect, the shell of a first subset of the plurality of microneedles is selected to degrade at a first time to release the therapeutic agent therein into skin of the subject, and wherein the shell of a second subset of the plurality of microneedles is selected to degrade at a second time to release the therapeutic agent therein into the skin of the subject. In another aspect, the shell of a first subset of the plurality of microneedles is selected to degrade at a first time to release a first dose of the vaccine therein into skin of the subject, and wherein the shell of a second subset of the plurality of microneedles is selected to degrade at a second time to release a second dose of the vaccine therein into the skin of the subject. In another aspect, the core of at least one of the microneedles includes a maximum diameter of about 200 μm and a maximum height of about 300 μm. In another aspect, at least one of the microneedles in the plurality of microneedles includes a maximum height of about 600 μm.

Another embodiment is the use of the vaccine delivery system described herein for administering a vaccine to a subject in need thereof.

Another embodiment described herein is a method for administering a vaccine to a subject in need thereof, the method comprising: contacting a vaccine delivery system with skin of the subject, the vaccine delivery system comprising: an assembly comprising: a substrate, and a plurality of microneedles extending from the substrate, each microneedle comprising: a shell comprising a biodegradable polymer, a core comprising a vaccine agent, and a cap comprising a biodegradable polymer, the cap enclosing the core within the shell; wherein the shell of each of the plurality of microneedles is selected to degrade at a predetermined time to release the vaccine agent therein into the skin of the subject. In one aspect, the vaccine agent is a protein or a nucleic acid. In another aspect, the shell of a first subset of the plurality of microneedles is selected to degrade at a first time to release the therapeutic agent therein into the skin of the subject, and wherein the shell of a second subset of the plurality of microneedles is selected to degrade at a second time to release the therapeutic agent therein into the skin of the subject. In another aspect, the shell of a first subset of the plurality of microneedles is selected to degrade at a first time to release a first dose of the vaccine therein into the skin of the subject, and wherein the shell of a second subset of the plurality of microneedles is selected to degrade at a second time to release a second dose of the vaccine therein into the skin of the subject.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A stabilized RNA vaccine composition, the composition comprising:
   a complex of RNA with one or more cationic polymers; and
   one or more cationic lipid entities.
Clause 2. The composition of clause 1, wherein the RNA comprises mRNA, miRNA, siRNA, tRNA, rRNA, or other RNA.
Clause 3. The composition of clause 1 or 2, wherein the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof.
Clause 4. The composition any one of clauses 1-3, wherein the cationic polymer comprises protamine.
Clause 5. The composition of any one of clauses 1-4, wherein the one or more cationic lipid entities comprises a liposome or lipid nanoparticles.
Clause 6. The composition of any one of clauses 1-5, wherein the liposome or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), di palmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof.
Clause 7. The composition of any one of clauses 1-6, further comprising one or more cryoprotectants.
Clause 8. The composition of any one of clauses 1-7, wherein the one or more cryoprotectants comprises one or more of trehalose, mannitol, sucrose, glucose, fructose, lactose, dextran, glycerol, ethylene glycol, propylene glycol, polyethylene glycol, or combinations thereof.
Clause 9. The composition of any one of clauses 1-8, further comprising one or more amino acids selected from lysine, arginine, histidine, glutamine, proline, glycine, threonine, or combinations thereof.
Clause 10. The composition of any one of clauses 1-9, wherein the complex of RNA with one or more cationic peptides has a size ranging from about 20 nm to about 500 nm.
Clause 11. The composition of any one of clauses 1-10, wherein the cationic lipid entity has a size ranging from about 20 nm to about 500 nm.
Clause 12. The composition of any one of clauses 1-11, wherein the stabilized RNA is thermally stable for at least about 2 months at about 37° C.
Clause 13. The composition of any one of clauses 1-12, wherein the stabilized RNA is thermally stable for at least about 2 hours at about 80° C.
Clause 14. A method for stabilizing RNA, the method comprising:
   (a) forming a complex comprising the RNA with one or more cationic peptides;
   (b) mixing the complex with one or more cationic lipid entities comprising liposomes or lipid nanoparticles to form a lipid mixture; and
   (c) drying the lipid mixture under vacuum.
Clause 15. The method of clause 14, wherein the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof.
Clause 16. The method of clause 14 or 15, wherein the liposome or lipid nanoparticles comprises one or more of dioleoyl-3-trimethylammonium propane (DOTAP), cholesterol, di methylaminoethane-carbamoyl cholesterol hydrochloride (DC-Cholesterol), dioleoyl-3-trimethylammonium propane (DOTMA), distearoylphosphatidylcholine (DSPC), di palmitoylphosphatidylcholine (DOPC), DSPE-PEG(2000) amine, Poly(ethylene glycol) dimethyl ether (PEG-DME), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof.
Clause 17. The method of any one of clauses 14-16, further comprising mixing the lipid mixture with one or more cryoprotectants comprising one or more of, trehalose, mannitol, sucrose, glucose, fructose, lactose, dextran, glycerol, ethylene glycol, propylene glycol, polyethylene glycol, or combinations thereof prior to drying the lipid mixture.
Clause 18. The method of any one of clauses 14-17, further comprising mixing the lipid mixture with one or more amino acids selected from lysine, arginine, histidine, glutamine, proline, glycine, threonine, or combinations thereof prior to drying the lipid mixture.
Clause 19. A stabilized RNA vaccine prepared by the method of any one of clauses 14-18.
Clause 20. Use of a stabilized RNA prepared by the method of any one of clauses 14-18 in the preparation of a vaccine medicament.
Clause 21. A stabilized vaccine agent composition, the composition comprising:
   a vaccine agent; and one or more cryoprotectants comprising one or more of trehalose, mannitol, sucrose, glucose, fructose, lactose, dextran, glycerol, ethylene glycol, propylene glycol, polyethylene glycol, or combinations.
Clause 22. The composition of clause 21, wherein the one or more cryoprotectants have a concentration of about 0.3 M to about 1 M.
Clause 23. The composition of clause 21 or 22, wherein the vaccine agent and the one or more cryoprotectants are present in a ratio ranging from about 1:1 (v/v) to about 1:6 (v/v).
Clause 24. The composition of any one of clauses 21-23, wherein the vaccine agent is a protein, functional fragment thereof, or combination thereof.
Clause 25. The composition of any one of clauses 21-24, wherein the composition is dried by lyophilization, vacuum, or dessication.
Clause 26. The composition of any one of clauses 21-25, wherein the vaccine agent is thermally stable for at least about 4 months at about 37° C.
Clause 27. The composition of any one of clauses 21-26, wherein the vaccine agent is thermally stable for at least about 1 hour at about 100° C.
Clause 28. A method for preparing a stabilized vaccine agent core, the method comprising:
   (a) mixing a vaccine agent with one or more cryoprotectants comprising one or more of trehalose, mannitol, sucrose, glucose, fructose, lactose, dextran, glycerol, ethylene glycol, propylene glycol, polyethylene glycol, or combinations thereof; and
   (b) drying the mixture.
Clause 29. The method of clause 28, wherein t one or more cryoprotectants have a concentration of about 0.3 M to about 1 M prior to drying.
Clause 30. The method of clause 28 or 29, wherein the vaccine agent is mixed with the one or more cryoprotectants at a ratio ranging from about 1:1 (v/v) to about 1:6 (v/v) prior to drying.
Clause 31. The method of any one of clauses 28-30, wherein the vaccine agent is a protein, functional fragment thereof, or combination thereof.
Clause 32. The method of any one of clauses 28-31, wherein drying in step (b) is performed by lyophilization, vacuum, or dessication.
Clause 33. The method of any one of clauses 28-32, wherein drying in step (b) is performed for at least about 4 hours to about 24 hours.
Clause 34. A stabilized vaccine core prepared by the method of any one of clauses 28-33.
Clause 35. Use of a stabilized vaccine core prepared by the method of any one of clauses 28-33 in the preparation of a vaccine medicament.
Clause 36. A method for one-step loading a vaccine core into a microneedle, the method comprising:
   (a) generating a core-shell microneedle assembly comprising a polymeric shell by compression-molding a film of poly(D,L-lactide-co-glycolide) (PLGA) into a silicone or poly(dimethylsiloxane) (PDMS) mold to form an assembly of microneedle shells, wherein the mold has an array of conical shaped cavities;
   (b) generating a vaccine core array by combining a vaccine agent with a cryoprotectant and casting the vaccine core on a second poly(dimethylsiloxane) (PDMS) mold having the same array of conical shaped cavities as the microneedle mold and drying the vaccine core;
   (c) transferring the vaccine core array onto a PLGA film;
   (d) aligning the vaccine core array on the PLGA film with the microneedle assembly, thereby filling the cores in the microneedle shell assembly with the vaccine cores; and
   (e) curing the microneedle assembly by vacuum heating to encapsulate the vaccine cores within the microneedles.
Clause 37. The method of clause 36, wherein generating a core-shell microneedle structure further comprises: removing an excess PLGA scum layer.
Clause 38. The method of clause 36 or 37, wherein the microneedles have a height of about 600 μm and a base diameter of about 300 μm.
Clause 39. The method of any one of clauses 36-38, wherein the vaccine cores have a height of about 400 μm and a diameter of about 200 μm.
Clause 40. A one-step loaded microneedle manufactured by the method of any one of clauses 36-39.
Clause 41. Use of a one-step loaded microneedle manufactured by the method of any one of clauses 36-39 to deliver a vaccine to a subject in need thereof.
Clause 42. A method for powder-filling a microneedle assembly, the method comprising:
   (a) preparing a poly(dimethylsiloxane) (PDMS) mold having an array of conical shaped cavities on a silicon wafer;
   (b) dispersing a powdered vaccine agent into the mold cavities;
   (c) compressing the powdered vaccine agent into the mold cavities;
   (d) repeating steps (b) and (c) until the microneedle cavities are filled;
   (e) casting a layer of one or more sugars and water-soluble polymer over the filled cavities to generate a microneedle assembly; and
   (f) drying the microneedle assembly.
Clause 43. The method of clause 42, wherein steps (b)-(d) are performed in a neutralized static environment.
Clause 44. The method of clause 42 or 43, wherein the vaccine agent loading capacity is about 20-fold greater than solution-casting core filling.
Clause 45. The method of any one of clauses 42-44, wherein the vaccine agent is a protein, a nucleic acid, or a combination thereof.
Clause 46. A filled microneedle assembly manufactured by the method of any one of clauses 42-45.
Clause 47. Use of a filled microneedle assembly manufactured by the method of any one of clauses 42-45 to deliver a vaccine to a subject in need thereof.
Clause 48. A method for manufacturing a microneedle assembly, the method comprising:
   (a) generating a core microneedle assembly by:
      filling a first silicone mold including a plurality of microneedle cavities with a copolymer,
      spinning the first silicone mold to remove a first scum layer of the copolymer on the first silicone mold, and
      generating a core in each of the copolymer-filled cavities;
   (b) generating a therapeutic agent assembly by:
      filling a plurality of cavities in a second silicone mold with a therapeutic agent,
      spinning the second silicone mold to remove a second scum layer of the therapeutic agent, and
      removing the molded therapeutic agent from the second mold and transferring the molded therapeutic agent to a substrate; and
   (c) aligning the therapeutic agent assembly with the core microneedle assembly to thereby fill the cores in the core microneedle assembly with the therapeutic agent.
Clause 49. The method of clause 48, wherein the cavities in the first silicone mold and the second silicone mold are conical shaped.
Clause 50. The method of clause 48 or 49, wherein the copolymer is poly(D,L-lactide-co-glycolide) (PLGA).
Clause 51. The method of any one of clauses 48-50, further comprising generating the first silicone mold by pouring polydimethylsiloxane (PDMS) over a master structure and curing.
Clause 52. The method of any one of clauses 48-51, wherein the vaccine agent is a protein or a nucleic acid.
Clause 53. The method of any one of clauses 48-52, further comprising generating a cap assembly by:
   (a) filling a plurality of cavities in a third silicone mold with a copolymer to form a plurality of caps;
   (b) spinning the third silicone mold to remove a third scum layer of the copolymer on the third silicone mold;
   (c) removing the molded caps from the third mold and transferring the molded caps onto the core microneedle assembly; and
   (d) aligning the cap assembly with the core microneedle assembly to thereby cover the cores in the core microneedle assembly with the caps.
Clause 54. The method of any one of clauses 48-53, further comprising applying heat to bond the cap assembly to the core microneedle assembly.
Clause 55. The method of any one of clauses 48-54, further comprising coating a supporting array with a water-soluble polymer and contacting the supporting array with the cap assembly.
Clause 56. The method of any one of clauses 48-55, further comprising applying heat to bond the supporting array to the cap assembly for removing the microneedle assembly from the first silicone mold.
Clause 57. A microneedle device, manufactured by the method of any one of clauses 48-56.
Clause 58. Use of a microneedle device, manufactured by the method of any one of clauses 48-56 for the delivery of a vaccine to a subject in need thereof.
Clause 59. A pulsatile vaccine delivery system comprising:
   a microneedle assembly including a plurality of microneedles filled with a vaccine agent;
   the microneedles comprising biodegradable polymers; and
   the microneedle assembly configured to release the vaccine agent at predetermined times with a predetermined amount of the vaccine agent while the microneedle assembly remains embedded in a subject.
Clause 60. The system of clause 59, wherein the biodegradable polymer degrades over time to release the therapeutic agent into the subject.
Clause 61. The system of clause 59 or 60, further comprising a computer processor in electronic communication with the microneedle assembly, the computer processor programmed with computer readable instructions to initiate the release of the vaccine agent.
Clause 62. The system of any one of clauses 59-61, further comprising an activator in electronic communication with the computer processor to initiate the release of the therapeutic agent.
Clause 63. The system of any one of clauses 59-62, wherein each of the microneedles in the plurality of microneedles is conical shaped.
Clause 64. The system of any one of clauses 59-63, wherein the vaccine agent is a protein or a nucleic acid.
Clause 65. The system of any one of clauses 59-64, wherein at least one of the microneedles in the plurality of microneedles includes a maximum height of about 600 μm.
Clause 66. Use of the pulsatile vaccine delivery system of any one of clauses 59-65 for the delivery of a vaccine to a subject in need thereof.
Clause 67. A vaccine delivery system comprising:
   an assembly comprising:
   (a) a substrate, and
   (b) a plurality of microneedles extending from the substrate, each microneedle comprising:
      a shell comprising a biodegradable polymer,
      a core comprising a vaccine agent, and
      a cap comprising a biodegradable polymer, the cap enclosing the core within the shell;
   wherein the shell of each of the plurality of microneedles is selected to degrade at a predetermined time to release the vaccine agent therein into skin of a subject.
Clause 68. The vaccine delivery system of clause 67, wherein the biodegradable polymer comprises poly(D,L-lactide-co-glycolide) (PLGA) or poly-lactide acid (PLA).
Clause 69. The vaccine delivery system of clause 67 or 68, wherein the cap comprises PLGA or PLA.
Clause 70. The vaccine delivery system of any one of clauses 67-69, wherein the shell of a first subset of the plurality of microneedles is selected to degrade at a first time to release the therapeutic agent therein into skin of the subject, and wherein the shell of a second subset of the plurality of microneedles is selected to degrade at a second time to release the therapeutic agent therein into the skin of the subject.
Clause 71. The vaccine delivery system of any one of clauses 67-70, wherein the shell of a first subset of the plurality of microneedles is selected to degrade at a first time to release a first dose of the vaccine therein into skin of the subject, and wherein the shell of a second subset of the plurality of microneedles is selected to degrade at a second time to release a second dose of the vaccine therein into the skin of the subject.
Clause 72. The vaccine delivery system of any one of clauses 67-71, wherein the core of at least one of the microneedles includes a maximum diameter of about 200 μm and a maximum height of about 300 μm.
Clause 73. The vaccine delivery system of any one of clauses 67-72, wherein at least one of the microneedles in the plurality of microneedles includes a maximum height of about 600 μm.
Clause 74. Use of the vaccine delivery system of any one of clauses 67-73 for administering a vaccine to a subject in need thereof.
Clause 75. A method for administering a vaccine to a subject in need thereof, the method comprising:
   contacting a vaccine delivery system with skin of the subject, the vaccine delivery system comprising:
      an assembly comprising:
      a substrate, and
      a plurality of microneedles extending from the substrate, each microneedle comprising:
      a shell comprising a biodegradable polymer,
      a core comprising a vaccine agent, and
      a cap comprising a biodegradable polymer, the cap enclosing the core within the shell;
   wherein the shell of each of the plurality of microneedles is selected to degrade at a predetermined time to release the vaccine agent therein into the skin of the subject.
Clause 76. The method of clause 75, wherein the vaccine agent is a protein or a nucleic acid.
Clause 77. The method of clause 75 or 76, wherein the shell of a first subset of the plurality of microneedles is selected to degrade at a first time to release the therapeutic agent therein into the skin of the subject, and wherein the shell of a second subset of the plurality of microneedles is selected to degrade at a second time to release the therapeutic agent therein into the skin of the subject.
Clause 78. The method of any one of clauses 75-77, wherein the shell of a first subset of the plurality of microneedles is selected to degrade at a first time to release a first dose of the vaccine therein into the skin of the subject, and wherein the shell of a second subset of the plurality of microneedles is selected to degrade at a second time to release a second dose of the vaccine therein into the skin of the subject.

### EXAMPLES

### Example 1

### Microneedle Fabrication and Loading Capacity

Fabrication methods have previously been reported to achieve core-shell microneedles (MNs) having unique release kinetics to simulate an immunogenic effect of multiple vaccine shots. These MNs have two components of a PLGA shell and a vaccine core having a vaccine of interest and a highly hydrophilic matrix of excipients (e.g., trehalose, sucrose) or polyvinylpyrrolidone (PVP), which are all used extensively in the drug and food industries with excellent safety profiles. FIG. 1D shows typical images of previously reported vaccine MNs. These MNs were created by micro-molding processes which are applied to produce the MNs of the present disclosure for immediate release of mRNA vaccines. An assessment of the maximal loading capacity of previous MNs (300 μm in base diameter; 600 μm in height; and patch size of 1×1 cm²) was also conducted (FIG. 1E). Two different methods were used to load vaccine antigen (using IgG protein as a model) into the MN core. First, the protein solution was casted into the MN mold and vacuum-dried with buffer to obtain a vaccine core (i.e., solution loading method). Second, the dried protein powder (or particles) was compressed into the MN mold (i.e., powder loading method) and the scum layer was removed by spinning water on the top. It was discovered that a significantly high and consistent amount of the protein (quantified by ELISA) loaded inside the MN patch via the powder loading method could be achieved. As shown in FIG. 1 E, per one patch of 1×1 cm², up to 4.5 mg of the protein antigen could be loaded along with 1.2 mg of excipients, including sucrose and trehalose at a 1:1 w/w ratio. As such, mRNA vaccine MNs can easily be generated with a much lower dose of 80 μg. The high loading capacity of MNs will allow for the incorporation of the mRNA with any other excipients and formulating agents to obtain high mRNA stability and bioactivity.

### Example 2

### Formulation and Stabilization of mRNA Vaccines

To develop formulations for stabilizing mRNA, mRNA is first used to encode the luciferase protein as an mRNA model for the study of thermal stability. See Table 1, (SEQ ID NO: 3). When added with luciferin, the luciferase protein emits bioluminescence and the transfection efficiency and subsequent mRNA bioactivity can be tested by performing an in vitro assay on cultured cells (including dendritic cells, muscle cells, and adipose stem cells which are common cells in the skin). Stabilizing excipients of sucrose and trehalose, which are known cryoprotectants, will be used to stabilize lipid nanoparticles (LNPs). For the mRNA, cationic protamine will be employed, which can bind to mRNA via the electrostatic force to prevent the molecule from flipping around and being denatured by self-hydrolysis (FIG. 1C).

TABLE 1
Firefly (Photinus pyralis) Luciferase mRNA (SEQ ID NO: 3)(All Thymidines
represent Uridines)

ATGGAGGACGCCAAGAACATCAAGAAGGGCCCCGCCCCCTTCTACCCCCTGGAGGACGGCACCGCCGGCGAGCAGC
TGCACAAGGCCATGAAGCGGTACGCCCTGGTGCCCGGCACCATCGCCTTCACCGACGCCCACATCGAGGTGGACAT
CACCTACGCCGAGTACTTCGAGATGAGCGTGCGGCTGGCCGAGGCCATGAAGCGGTACGGCCTGAACACCAACCAC
CGGATCGTGGTGTGCAGCGAGAACAGCCTGCAGTTCTTCATGCCCGTGCTGGGCGCCCTGTTCATCGGCGTGGCCG
TGGCCCCCGCCAACGACATCTACAACGAGCGGGAGCTGCTGAACAGCATGGGCATCAGCCAGCCCACCGTGGTGTT
CGTGAGCAAGAAGGGCCTGCAGAAGATCCTGAACGTGCAGAAGAAGCTGCCCATCATCCAGAAGATCATCATCATG
GACAGCAAGACCGACTACCAGGGCTTCCAGAGCATGTACACCTTCGTGACCAGCCACCTGCCCCCCGGCTTCAACG
AGTACGACTTCGTGCCCGAGAGCTTCGACCGGGACAAGACCATCGCCCTGATCATGAACAGCAGCGGCAGCACCGG
CCTGCCCAAGGGCGTGGCCCTGCCCCACCGGACCGCCTGCGTGCGGTTCAGCCACGCCCGGGACCCCATCTTCGGC
AACCAGATCATCCCCGACACCGCCATCCTGAGCGTGGTGCCCTTCCACCACGGCTTCGGCATGTTCACCACCCTGG
GCTACCTGATCTGCGGCTTCCGGGTGGTGCTGATGTACCGGTTCGAGGAGGAGCTGTTCCTGCGGAGCCTGCAGGA
CTACAAGATCCAGAGCGCCCTGCTGGTGCCCACCCTGTTCAGCTTCTTCGCCAAGAGCACCCTGATCGACAAGTAC
GACCTGAGCAACCTGCACGAGATCGCCAGCGGCGGCGCCCCCCTGAGCAAGGAGGTGGGCGAGGCCGTGGCCAAGC
GGTTCCACCTGCCCGGCATCCGGCAGGGCTACGGCCTGACCGAGACCACCAGCGCCATCCTGATCACCCCCGAGGG
CGACGACAAGCCCGGCGCCGTGGGCAAGGTGGTGCCCTTCTTCGAGGCCAAGGTGGTGGACCTGGACACCGGCAAG
ACCCTGGGCGTGAACCAGCGGGGCGAGCTGTGCGTGCGGGGCCCCATGATCATGAGCGGCTACGTGAACAACCCCG
AGGCCACCAACGCCCTGATCGACAAGGACGGCTGGCTGCACAGCGGCGACATCGCCTACTGGGACGAGGACGAGCA
CTTCTTCATCGTGGACCGGCTGAAGAGCCTGATCAAGTACAAGGGCTACCAGGTGGCCCCCGCCGAGCTGGAGAGC
ATCCTGCTGCAGCACCCCAACATCTTCGACGCCGGCGTGGCCGGCCTGCCCGACGACGACGCCGGCGAGCTGCCCG
CCGCCGTGGTGGTGCTGGAGCACGGCAAGACCATGACCGAGAAGGAGATCGTGGACTACGTGGCCAGCCAGGTGAC
CACCGCCAAGAAGCTGCGGGGCGGCGTGGTGTTCGTGGACGAGGTGCCCAAGGGCCTGACCGGCAAGCTGGACGCC
CGGAAGATCCGGGAGATCCTGATCAAGGCCAAGAAGGGCGGCAAGATCGCCGTGTGA
CleanCap® FLuc mRNA, TriLink BioTechnologies, LLC, LLC; Catalog No. L-7202 (Substituted with 5-methoxyuridine); see www.trilinkbiotech.com/cleancap-fluc-mrna-5mou.html

Previous reports have provided insight into forming the particles of liposomes or LNP-mRNA-protamine lipid particles. First, the complex of mRNA and protamine is formed. Next, mRNA is obtained that encodes the luciferase protein or other proteins of interest (e.g., S-proteins of COVID-19 virus or alternative antigens for other vaccines) from a commercial source (e.g.,

TriLink Inc.) and the mRNA is mixed with protamine inside a buffer. The cationic protamine and mRNA self-assemble into the complex particles after 20-30 minutes of incubation at room temperature. The ratio between protamine and mRNA (w/w) can be varied to obtain different particle sizes in the range of about 20-500 nm.

To form the liposome particles, a lipid solution of DOTAP (a cationic lipid), cholesterol, and DSPE-PEG2000 amine is first prepared at a molar ratio of 10:10:1 in the organic solvent of chloroform and methanol (9:1, v/v). Next, the solvent is removed to obtain a solid lipid film. The film is then rehydrated in the buffer and mixed with the mRNA-protamine complex under mechanical stirring to form the liposome particles containing the mRNA-protamine complex. The resultant solution is then mixed with excipients including sugar (e.g., trehalose, sucrose, etc.), which have been shown to stabilize lipid nanoparticles at high storage temperatures. During the synthesis process, different formulations can be obtained by varying the ratio of lipids vs. protamine vs. mRNA and the ratio of excipients vs. the liposome particles. Finally, the lipid-mRNA-protamine particles and the excipient solutions are dried under vacuum at room temperature to mimic the process of microneedle (MN) fabrication and are then used for the assessment of thermal stability.

To form the LNP-mRNA-protamine particles, a similar process is followed except that PEG-DME, cholesterol, DLin-MC3, and DOPE are used as the lipids. The cationic protamine and mRNA self-assemble into the complex particles after about 10-30 minutes of incubation. PEG-DME, cholesterol, DLin-MC3, and DOPE are soluble and mixed in ethanol and are rapidly added to the mRNA-protamine solution. After about 30 minutes of incubation, the ethanol is removed by dialysis against PBS, providing the final LNP-mRNA-protamine particles.

In each step of the process for synthesizing the lipid-protamine-mRNA particles, particle formation is inspected by dynamic light scattering (DLS) to quantify the particle size and distribution. Electrophoresis is also performed to confirm the stability of mRNA (if mRNA degrades, it will break into an array of bands in the electrophoresis gel, compared with the stock mRNA), the binding between mRNA and protamine, and the formation of the lipid particles containing mRNA and protamine.

### Example 3

### Lipid Nanoparticles with mRNA-Protamine Complex Show Enhanced Cell Transfection In Vitro

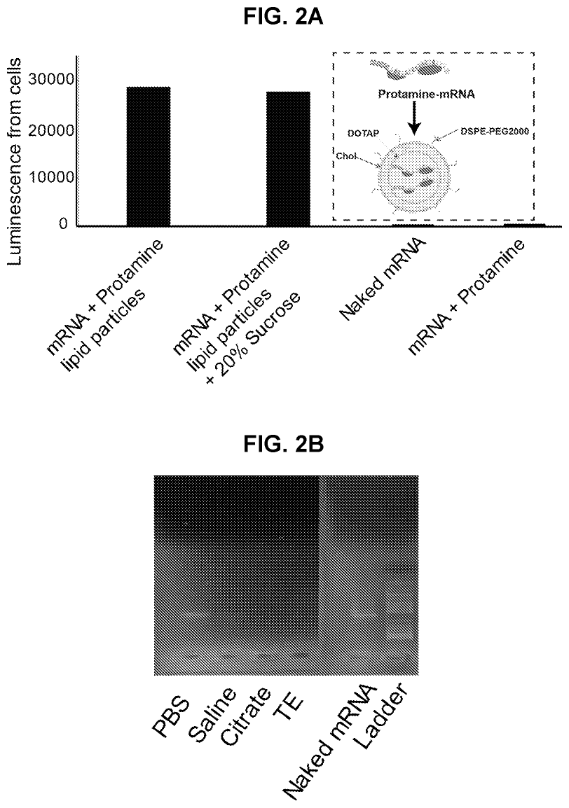

Previous works have shown that a cationic protein of protamine can bind with mRNA and significantly increase the mRNA stability, as a complex of mRNA-protamine was stable even when stored at 70° C. for several months. However, the complex alone has been reported to not be effective for transfecting cells typically present in the subcutaneous or intramuscular areas. As such, mRNA complexes need to be further encapsulated inside lipid or liposome particles to enhance cellular transfection. Such a formulation has been generated to obtain the lipid-mRNA-protamine particles in a citrate buffer. FIG. 2A-B show that stabilized mRNA (i.e., mRNA-protamine complex) contained inside lipid particles helps to enhance stability and cell transfection as measured by luciferase luminescence assays and gel electrophoresis. Sucrose was also added to stabilize the lipid nanoparticles. As shown in FIG. 2A, these lipid-mRNA-protamine particles could transfect cells (adipose stem cells) to highly express the luciferase protein, as quantified by bioluminescence signal. In contrast, the mRNA-protamine complex alone was not effective in transfecting the cells. It was further confirmed using electrophoresis gels that the complex mRNA-protamine was generated (FIG. 2B). The protamine and mRNA were mixed in different buffers (PBS, saline, citrate, and TE buffer) and electrophoresis was performed, using naked mRNA as the control. As shown in FIG. 2B, it was found that while the mRNA and protamine were not bonded well in PBS and saline, the citrate and TE buffer facilitated the bonding to display no naked mRNA band in the gel.

These data suggest that the lipid shell is needed to facilitate the transfection of cells by using the mRNA-protamine complex. As protamine is an excellent stabilizer for mRNA, this formulation offers a good platform to stabilize mRNA while still enabling the mRNA to effectively transfect cells for protein expression. As such, different lipid-mRNA-protamine nanoparticles (with different ratios of lipid vs. protamine vs. mRNA vs. excipients) can be formulated to test the stability of these particles under heat and test the drying process in MN fabrication.

### Example 4

### Heat Stability of mRNA (RNA) and mRNA-Protamine Complex (RNA-Pro) with and without Sugar

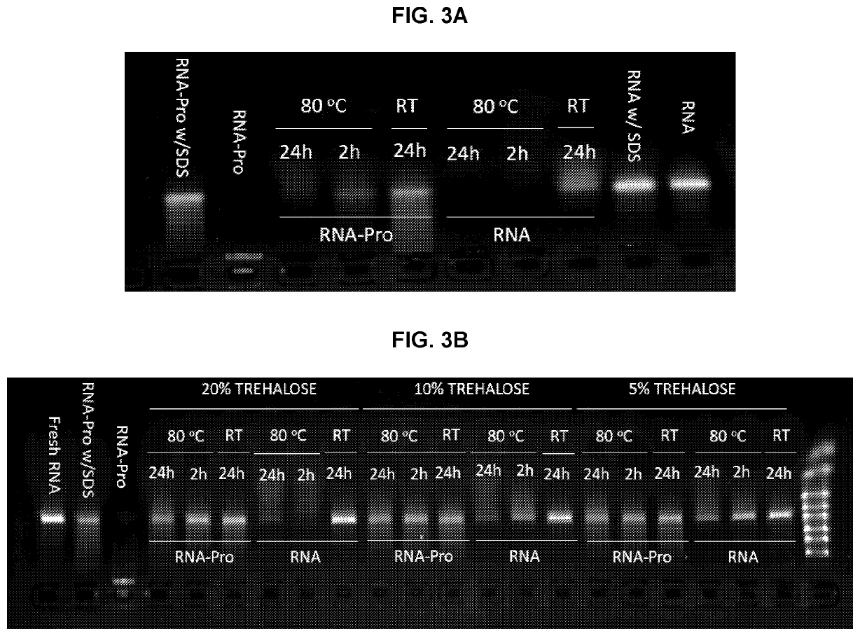

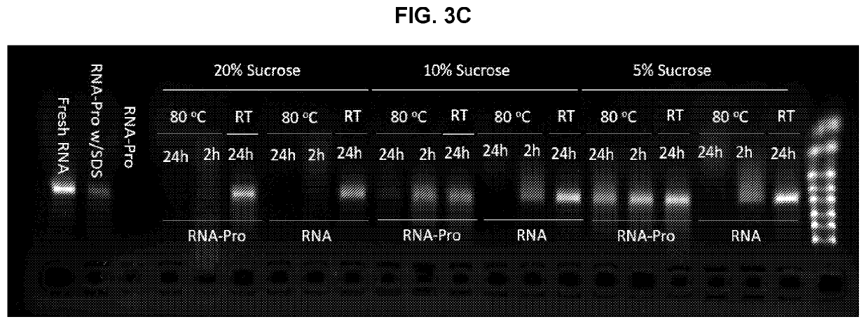

The heat stability of mRNA (RNA) and mRNA-protamine complex (RNA-Pro) with and without different sugar excipients was investigated using gel electrophoresis. For testing the stability without sugar, each sample in the RNA-Pro group (3 samples in total), mRNA (100 ng) was incubated with protamine (100 ng) in TE buffer (pH 7.2) for 30 min at room temperature (RT). For each sample in the RNA group, free mRNA (100 ng) in TE buffer was also incubated at RT for 30 min. Water was then removed in each group under vacuum (24 hr at RT). Next, samples of RNA and RNA-Pro were either exposed to 80° C. for 2 hr and 24 hr, or placed at RT for 24 hr. Finally, 3.75% of sodium dodecyl sulfate (SDS) in Milli-Q water was added to redissolve RNA or RNA-Pro complex and further dissociate mRNA from protamine. Upon the addition of gel loading dye, samples were analyzed on a 1.6% agarose gel in RNase free Tris-Borate-EDTA (TBE) buffer. FIG. 3A shows that in the absence of any sugar excipient: (1) the protamine retards the electrophoresis of mRNA in the gel; (2) the addition of SDS can dissociate the mRNA from the mRNA-protamine complex; (3) the addition of SDS did not affect the electrophoretic property of free mRNA; and (4) compared to free mRNA after vacuum drying, the RNA-Pro complex shows better heat resistance at 80° C.

The heat stability of RNA and RNA-Pro was then tested with different concentrations of trehalose. For each sample in the RNA-Pro group, mRNA (100 ng) was incubated with protamine (100 ng) in TE buffer (pH 7.2) for 30 min at RT. For each sample in the RNA group, free mRNA (100 ng) in TE buffer was also incubated at RT for 30 min. Then, different concentrations of trehalose in TE buffer were added to the two groups, resulting in 5%, 10%, or 20% trehalose. All samples were then subjected to reduced pressure to remove water by vacuum (24 hr at RT). Samples of RNA and RNA-Pro were either exposed to 80° C. for 2 hr and 24 hr, or placed at RT for 24 hr. At 24 hr, samples were withdrawn and 28 μL of 3.75% SDS in Milli-Q water was added to redissolve RNA or RNA-Pro complex and further dissociate mRNA from protamine. Upon the addition of gel loading dye, samples were analyzed on a 1.6% agarose gel in RNase free Iris-Borate-EDTA (TBE) buffer. FIG. 3B shows that: (1) the protamine retarded the electrophoresis of mRNA in the gel; (2) the addition of SDS can dissociate the mRNA from mRNA-protamine complex; (3) compared to conditions with no sugar, the addition of trehalose can significantly improve the heat stability of mRNA in all groups, with even better efficacy in protecting the mRNA in an RNA-protamine complex; and (4) higher concentrations of trehalose (i.e., 20% trehalose) seems to provide mRNA in RNA-protamine complexes with better heat protection.

The heat stability of RNA and RNA-Pro was then tested with different concentrations of sucrose. For each sample in the RNA-Pro group, mRNA (100 ng) was incubated with protamine (100 ng) in TE buffer (pH 7.2) for 30 min at RT. For each sample in the RNA group, free mRNA (100 ng) in TE buffer was also incubated at RT for 30 min. Then, different concentrations of sucrose in TE buffer were added to the two groups, resulting in 5%, 10%, or 20% sucrose. All samples were then subjected to reduced pressure to remove water by vacuum (24 hr at RT). Samples of RNA and RNA-Pro were either exposed to 80° C. for 2 hr and 24 hr, or placed at RT for 24 hr. At 24 hr, samples were withdrawn and 28 μL of 3.75% SDS in Milli-Q water was added to redissolve RNA or RNA-Pro complex and further dissociate mRNA from protamine. Upon the addition of gel loading dye, samples were analyzed on a 1.6% agarose gel in RNase free Tris-Borate-EDTA (TBE) buffer. FIG. 3C shows that: (1) the protamine retarded the electrophoresis of mRNA in the gel; (2) the addition of SDS can dissociate the mRNA from mRNA-protamine complex; (3) the addition of sucrose can improve the heat stability of mRNA in all groups, compared to conditions with no sugar, after vacuum drying; (4) the samples of the mRNA-protamine complex group show higher heat stability when compared to the mRNA alone group; and (5) a lower concentration of sucrose (i.e., 5% sucrose) seems to provide the best heat protection for mRNA in RNA-protamine complex, compared to 10% or 20% sucrose.

### Example 5

### Stabilization and Assessment of mRNA Formulations In Vitro

The formulated mRNA is added with polyol or sugar excipients (e.g., trehalose, sucrose, mannitol, silk, gelatin, etc.) at different ratios and compositions. The mixture is then dried under vacuum to obtain dried mRNA formulations inside the excipient matrix.

Screening is performed to identify the dry formulations which can sustain mRNA bioactivity at high temperatures of about 65° C. to 75° C. for a short period of time (i.e., about 1 hr). Once the mRNAs can survive these conditions, they also can retain bioactivity after the MN fabrication process, which employs a temperature of about 40-60° C. for about 15-30 minutes. Then, the integrity of the mRNA is tested using SDS to detach the protamine and lipid nanoparticles (which can help to retrieve the free mRNA for inspection), and then electrophoresis gels are used to inspect the size of the mRNA.

Next, the formulations are incubated after being exposed to the heat with cultured cells (e.g., dendritic cells) and bioluminescent signals are quantified that are expressed by the transfected cells. The bioluminescent signal is compared to the freshly made lipid-mRNA-protamine particles before being exposed to the heat, and the LNP particles that only contain the mRNA as positive controls. In addition, naked mRNA (i.e., mRNA without lipid vehicles and the protamine complex) may also be used as a negative control for the cell transfection assay. The formulations may also be tested for transfection in other cell lines such as muscle cells and adipose stem cells.

Following the screening process, lead formulations were obtained for longer-term stability studies. Again, the vacuum-dried formulations were incubated at 37° C. for about 1 month. A one-month period was selected because it covers the two conventional shots of the mRNA rabies vaccine at days 0 and 21. Then, the dried vaccine formulations were rehydrated, and the cell transfection assay was performed to quantify the expression of luciferase in comparison with the control formulations which were not exposed to high temperature (i.e., freshly made samples).

### Example 6

### Lipid Nanoparticles (LNPs) Containing mRNA Have High Cell Transfection In Vitro

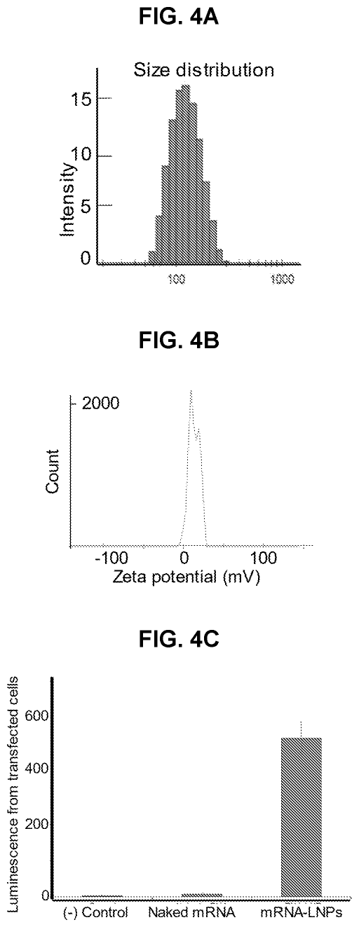

Freshly made LNPs containing mRNA were found to highly transfect cultured cells in vitro. The LNPs have a size range of about 100-200 nm and have an overall positive charge (FIG. 4A-B), which allows cells to easily uptake the particles. The freshly made LNPs significantly increased mRNA transfection and helped LN-229 cultured cells to express luciferase bioluminescence, while the control of naked mRNA alone expressed little-to-no optical signal (FIG. 4C). Therefore, a sample of freshly made LNP containing mRNA was used as a positive control to assess the stability and transfection of LNPs or liposome particles carrying stabilized mRNA that is complexed with protamine.

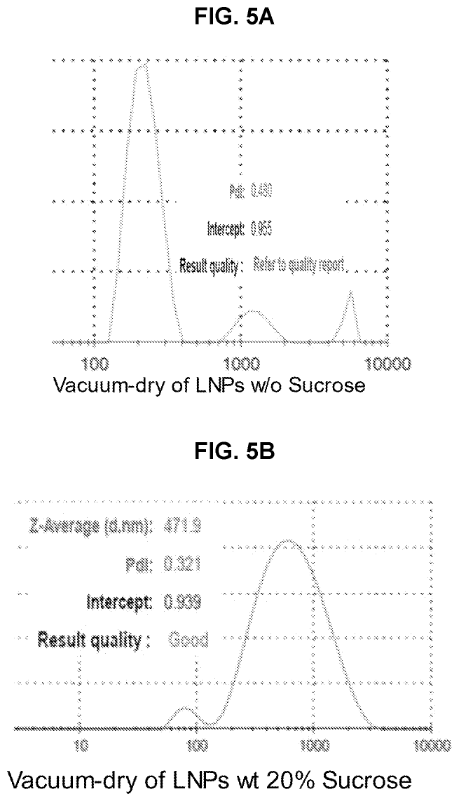

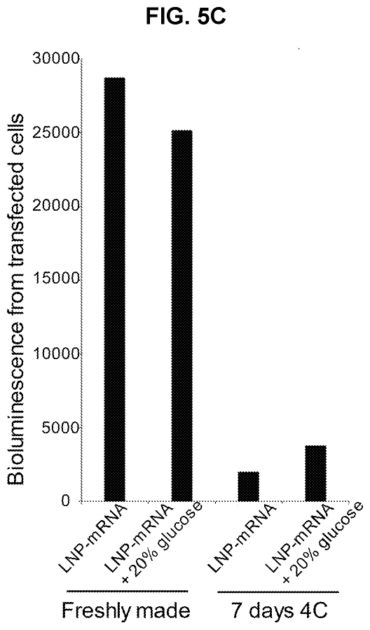

Additionally, sucrose and trehalose can stabilize the LNPs by retaining size and shape under high heat conditions. As shown in FIG. 5A of the DLS data for the formulation without excipients, the lipid particles were not stable under vacuum drying, which is a process used to fabricate MNs and create a vaccine core. Specifically, the particle sizes could not be determined under the DLS analysis after the particles were dried at room temperature under a high vacuum. However, when 20% sucrose was added, which is a cryoprotectant, the particles retain their size of about 470 nm under vacuum drying (FIG. 5B).

Furthermore, another test was performed on the cell transfection, again using the mRNA encoding the reporter protein of luciferase. Two formulations of LNP-mRNA were generated with and without the excipient sucrose. It was discovered that both samples of freshly made particles (i.e., prior to vacuum drying and heat exposure) with and without the excipient can perform an effective transfection of the cultured cells to exhibit the bioluminescence, although there is a slight reduction in bioluminescence for the sample with sucrose (FIG. 5C). In comparison, when vacuum-drying and storing the two formulations at 40° C. for 7 days, it was found that sucrose samples retained transfection activity more than samples without sucrose, although there was a large reduction in the bioluminescence activity from the freshly made samples (FIG. 5C).

Collectively, these data suggest that the addition of cryo-protectant excipients is helpful for the stability of LN Ps. However, the cryo-protectants alone cannot stabilize the mRNA, leading to low transfection activity. Based on this observation, particles need to be formulated with different concentrations of the cryo-protectants such as sucrose and trehalose, and protamine should also be used to stabilize the mRNA.

### Example 7

### Assessment of In Vivo Immunogenicity by Transdermal Delivery of Stabilized mRNA in Rats

Previous works have shown that stabilized mRNA-protamine complexes are not effective for hypodermic injections, while the use of transdermal devices allow the mRNA complex to highly transfect cells and express the protein of interest at the dermal layer. This result is most likely due to the presence of many dendritic Langerhan cells which can effectively uptake and translate the mRNA at the superficial skin layer.

This study is designed to assess how stabilized mRNA can effectively trigger in vivo immunogenicity via dermal delivery using simple dissolvable MNs with immediate release at time 0. It is hypothesized that via the use of MNs for dermal delivery, the stabilized mRNA vaccines can exhibit a good immunogenicity in rats that is superior to the immunogenic effect obtained from conventional hypodermic injection techniques.

Experiments will be performed to assess whether the mRNA formulations can sustain bioactivity after MN fabrication and heat exposure to induce antibody response in vivo. As such, a model of mRNA vaccine will be employed, which encodes the glycoprotein of rabies virus, to assess the immune response in animals. The mRNA encoding the sequence of rabies viral strain will be utilized (GenBank accession number: AAA47218.1; SEQ ID NO: 4-5). The rabies mRNA vaccine has previously been shown to produce immunogenic efficacy with two subcutaneous/intramuscular shots at days 0 and 21. As such, to stimulate a significant immunogenicity for antibody detection, a conventional prime-booster mRNA vaccination schedule will be employed where the dermal MNs will be administered at days 0 and 21 for comparison with the previously reported subcutaneous/intramuscular shots.

Exemplary mRNAs that can be used for vaccines using the method described herein are shown in Table 2.

TABLE 2
Exemplary Vaccine mRNAs (all Thymidines represent Uridines)
Rabies (Lyssavirus rabies)
Rabies transmembrane glycoprotein G (Gene bank: AAA47218.1)(SEQ ID NO: 4)
ATGGTTCCTCAGGCTCTCCTGTTTGTACCCCTTCTGGTTTTTCCATTGTGTTTTGGGAAATTCCCTATTTACACGA
TACCAGACAAGCTTGGTCCCTGGAGCCCGATTGACATACATCACCTCAGCTGCCCAAACAATTTGGTAGTGGAGGA
CGAAGGATGCACCAACCTGTCAGGGTTCTCCTACATGGAACTTAAAGTTGGATACATCTCAGCCATAAAAATGAAC
GGGTTCACTTGCACAGGCGTTGTGACGGAGGCTGAAACCTACACTAACTTCGTTGGTTATGTCACAACCACGTTCA
AAAGAAAGCATTTCCGCCCAACACCAGATGCATGTAGAGCCGCGTACAACTGGAAGATGGCCGGTGACCCCAGATA
TGAAGAGTCTCTACACAATCCGTACCCTGACTACCACTGGCTTCGAACTGTAAAAACCACCAAGGAGTCTCTCGTT
ATCATATCTCCAAGTGTGGCAGATTTGGACCCATATGACAGATCCCTTCACTCGAGGGTCTTCCCTGGCGGGAATT
GCTCAGGAGTAGCGGTGTCTTCTACCTACTGCTCCACTAACCACGATTACACCATTTGGATGCCCGAGAATCCGAG
ACTAGGGATGTCTTGTGACATTTTTACCAATAGTAGAGGGAAGAGAGCATCCAAAGGGAGTGAGACTTGCGGCTTT
GTAGATGAAAGAGGCCTATATAAGTCTTTAAAAGGAGCATGCAAACTCAAGTTATGTGGAGTTCTAGGACTTAGAC
TTATGGATGGAACATGGGTCGCGATGCAAACATCAAATGAAACCAAATGGTGCCCTCCCGGTCAGTTGGTGAATTT
GCACGACTTTCGCTCAGACGAAATTGAGCACCTTGTTGTAGAGGAGTTGGTCAAGAAGAGAGAGGAGTGTCTGGAT
GCACTAGAGTCCATCATGACCACCAAGTCAGTGAGTTTCAGACGTCTCAGTCATTTAAGAAAACTTGTCCCTGGGT
TTGGAAAAGCATATACCATATTCAACAAGACCTTGATGGAAGCCGATGCTCACTACAAGTCAGTCAGAACTTGGAA
TGAGATCATCCCTTCAAAAGGGTGTTTAAGAGTTGGGGGGAGGTGTCATCCTCATGTAAACGGGGTATTTTTCAAT
GGTATAATATTAGGACCTGACGGCAATGTCTTAATCCCAGAGATGCAATCATCCCTCCTCCAGCAACATATGGAGT
TGTTGGTATCCTCGGTTATCCCCCTTATGCACCCCCTGGCAGACCCGTCTACCGTTTTCAAGAACGGTGACGAGGC
TGAGGATTTTGTTGAAGTTCACCTTCCCGATGTGCACGAACGGATCTCAGGAGTTGACTTGGGTCTCCCGAACTGG
GGGAAGTATGTATTACTGAGTGCAGGGGCCCTGACTGCCTTGATGTTGATAATTTTCCTGATGACATGCTGGAGAA
GAGTCAATCGATCGGAACCTACACAACACAATCTCAGAGGGACAGGGAGGGAGGTGTCAGTCACTCCCCAAAGCGG
GAAGATCATATCTTCATGGGAATCATACAAGAGCGGGGGTGAGACCGGACTGTGA
Rabies transmembrane glycoprotein G - Sequence Optimized by AI (SEQ ID NO: 5)
ATGGTTCCTCAGGCTCTGCTGTTCGTGCCCCTGCTGGTGTTCCCTCTGTGCTTCGGCAAGTTCCCCATCTACACAA
TCCCCGACAAGCTCGGCCCTTGGAGCCCTATCGATATCCACCACCTGAGCTGCCCCAACAACCTGGTGGTGGAAGA
TGAGGGCTGCACCAACCTGAGCGGCTTCAGCTACATGGAACTGAAAGTGGGCTACATCAGCGCCATCAAGATGAAC
GGCTTCACCTGTACCGGCGTGGTCACAGAGGCCGAGACATACACAAACTTCGTGGGCTACGTGACCACCACCTTCA
AGAGAAAGCACTTCAGACCCACACCTGACGCCTGCAGAGCCGCCTACAATTGGAAGATGGCCGGCGATCCCAGATA
CGAGGAAAGCCTGCACAACCCCTATCCTGACTACCACTGGCTGAGAACCGTGAAAACCACCAAAGAGAGCCTGGTC
ATCATCAGCCCCAGCGTGGCAGACCTGGATCCTTACGACAGATCCCTGCACAGCAGAGTGTTCCCTGGCGGCAACT
GTAGCGGAGTGGCTGTGTCTAGCACCTACTGCAGCACCAACCACGACTACACCATCTGGATGCCTGAGAACCCCAG
ACTGGGCATGAGCTGCGACATCTTCACCAACTCCAGAGGCAAGAGAGCCAGCAAGGGCAGCGAGACATGTGGCTTC
GTGGATGAGAGAGGCCTGTACAAGTCTCTGAAGGGCGCCTGCAAGCTGAAGCTGTGTGGTGTCCTGGGCCTGAGAC
TGATGGATGGAACATGGGTCGCCATGCAGACCAGCAACGAGACAAAGTGGTGTCCACCTGGCCAGCTGGTCAACCT
GCACGACTTTAGAAGCGACGAGATCGAGCATCTGGTGGTCGAGGAACTGGTCAAGAAAAGGGAAGAGTGCCTGGAC
GCCCTGGAATCCATCATGACCACCAAGAGCGTGTCCTTCAGAAGGCTGAGCCACCTGAGAAAGCTGGTGCCCGGCT
TTGGCAAGGCCTATACCATCTTCAACAAGACCCTGATGGAAGCCGACGCTCACTACAAGTCTGTGCGGACCTGGAA
CGAGATCATCCCTAGCAAGGGCTGCCTGAGAGTCGGCGGAAGATGTCACCCTCATGTGAACGGCGTGTTCTTCAAC
GGCATCATCCTGGGACCTGACGGCAACGTGCTGATCCCTGAGATGCAGTCTAGCCTGCTCCAGCAGCATATGGAAC
TGCTCGTGTCCAGCGTGATCCCTCTGATGCACCCTCTGGCCGATCCTAGCACCGTGTTCAAGAATGGCGACGAGGC
CGAGGACTTCGTGGAAGTGCATCTGCCTGACGTGCACGAGAGAATCTCTGGCGTTGACCTGGGACTGCCCAACTGG
GGCAAATACGTGCTGCTTTCTGCCGGCGCTCTGACAGCCCTGATGCTGATCATCTTCCTGATGACCTGCTGGCGGA
GAGTGAACAGAAGCGAGCCCACACAGCACAACCTGAGAGGCACAGGCAGAGAAGTGTCTGTGACACCCCAGAGCGG
CAAGATCATCAGCAGCTGGGAGAGCTACAAGAGCGGCGGAGAGACAGGACTGTGA
Influenza
Alphainfluenzavirus virus (Influenza A/Puerto Rico/8/34(H1N1)) hemagglutinin (Gene bank:
ABO21709.1)(SEQ ID NO: 6)
ATGAAGGCAAACCTACTGGTCCTGTTATGTGCACTTGCAGCTGCAGATGCAGACACAATATGTATAGGCTACCATG
CGAACAATTCAACCGACACTGTTGACACAGTACTCGAGAAGAATGTGACAGTGACACACTCTGTTAACCTGCTCGA
AGACAGCCACAACGGAAAACTATGTAGATTAAAAGGAATAGCCCCACTACAATTGGGGAAATGTAACATCGCCGGA
TGGCTCTTGGGAAACCCAGAATGCGACCCACTGCTTCCAGTGAGATCATGGTCCTACATTGTAGAAACACCAAACT
CTGAGAATGGAATATGTTATCCAGGAGATTTCATCGACTATGAGGAGCTGAGGGAGCAATTGAGCTCAGTGTCATC
ATTCGAAAGATTCGAAATATTTCCCAAAGAAAGCTCATGGCCCAACCACAACACAAACGGAGTAACGGCAGCATGC
TCCCATGAGGGGAAAAGCAGTTTTTACAGAAATTTGCTATGGCTGACGGAGAAGGAGGGCTCATACCCAAAGCTGA
AAAATTCTTATGTGAACAAAAAAGGGAAAGAAGTCCTTGTACTGTGGGGTATTCATCACCCGCCTAACAGTAAGGA
ACAACAGAATCTCTATCAGAATGAAAATGCTTATGTCTCTGTAGTGACTTCAAATTATAACAGGAGATTTACCCCG
GAAATAGCAGAAAGACCCAAAGTAAGAGATCAAGCTGGGAGGATGAACTATTACTGGACCTTGCTAAAACCCGGAG
ACACAATAATATTTGAGGCAAATGGAAATCTAATAGCACCAATGTATGCTTTCGCACTGAGTAGAGGCTTTGGGTC
CGGCATCATCACCTCAAACGCATCAATGCATGAGTGTAACACGAAGTGTCAAACACCCCTGGGAGCTATAAACAGC
AGTCTCCCTTACCAGAATATACACCCAGTCACAATAGGAGAGTGCCCAAAATACGTCAGGAGTGCCAAATTGAGGA
TGGTTACAGGACTAAGGAACAATCCGTCCATTCAATCCAGAGGTCTATTTGGAGCCATTGCCGGTTTTATTGAAGG
GGGATGGACTGGAATGATAGATGGATGGTATGGTTATCATCATCAGAATGAACAGGGATCAGGCTATGCAGCGGAT
CAAAAAAGCACACAAAATGCCATTAACGGGATTACAAACAAGGTGAACACTGTTATCGAGAAAATGAACATTCAAT
TCACAGCTGTGGGTAAAGAATTCAACAAATTAGAAAAAAGGATGGAAAATTTAAATAAAAAAGTTGATGATGGATT
TCTGGACATTTGGACATATAATGCAGAATTGTTAGTTCTACTGGAAAATGAAAGGACTCTGGATTTCCATGACTCA
AATGTGAAGAATCTGTATGAGAAAGTAAAAAGCCAATTAAAGAATAATGCCAAAGAAATCGGAAATGGATGTTTTG
AGTTCTACCACAAGTGTGACAATGAATGCATGGAAAGTGTAAGAAATGGGACTTATGATTATCCCAAATATTCAGA
AGAGTCAAAGTTGAACAGGGAAAAGGTAGATGGAGTGAAATTGGAATCAATGGGGATCTATCAGATTCTGGCGATC
TACTCAACTGTCGCCAGTTCACTGGTGCTTTTGGTCTCCCTGGGGGCAATCAGTTTCTGGATGTGTTCTAATGGAT
CTTTGCAGTGCAGAATATGCATCTGA
Influenza A virus (Influenza A/Victoria/3/1975(H3N2)) hemagglutinin (GenBank: V01086.1)(SEQ ID NO:
7)
ATGAAGACTATCATTGCTTTGAGCTACATTTTCTGTCTGGTTTTCGCCCAAGACCTTCCAGGAAATGACAACAACA
GCACAGCAACGCTGTGCCTGGGACATCATGCGGTGCCAAACGGAACGCTAGTGAAAACAATCACGAATGATCAGAT
TGAAGTGACTAATGCTACTGAGCTGGTTCAGAGTTCCTCAACGGGTAAAATATGCAACAATCCTCATCGAATCCTT
GATGGAATAAACTGCACACTGATAGATGCTCTATTGGGGGACCCTCATTGTGATGGATTTCAAAATGAGAAATGGG
ACCTTTTCGTTGAACGCAGCAAAGCTTTCAGCAACTGTTACCCTTATGATGTGCCAGATTATGCCTCCCTTAGGTC
ACTAGTTGCCTCGTCAGGCACTCTGGAGTTTATCAATGAAGGCTTCAATTGGACTGGGGTCACTCAGAATGGGGGA
AGCAGTGCTTGCAAAAGAGGACCTGATAGCGGTTTTTTCAGTAGACTGAACTGGTTGTACAAATCAGGAAGCACAT
ATCCAGTGCAAAACGTGACCATGCCAAACAATGACAATTCTGACAAACTATACATTTGGGGGGTTCACCACCCGAG
CACGGACAAAGAACAAACCAACCTATATGTTCAAGCATCAGGGAAAGTCACAGTCTCCACCAAGAGAAGCCAGCAA
ACTATAATCCCGAATGTCGGGTCTAGACCCTGGGTAAGGGGTCTGTCTAGTAGAATAAGCATCTATTGGACAATAG
TTAAACCGGGAGACATACTGGTAATTAATAGTAATGGGAACCTAATTGCTCCTCGGGGTTACTTCAAAATGCGCAC
TGGGAAAAGCTCAATAATGAGGTCAGATGCACCTATTGGCACCTGCAGCTCTGAATGCATCACTCCAAATGGAAGC
ATTCCCAATGACAAGCCCTTTCAAAACGTAAACAAGATCACATATGGGGCATGTCCCAAGTATGTTAAGCAAAACA
CTCTGAAGTTGGCAACAGGGATGCGGAATGTACCAGAGAAACAAACTAGAGGCATATTCGGCGCAATAGCAGGTTT
CATAGAAAATGGTTGGGAGGGAATGATAGACGGTTGGTACGGTTTCAGGCATCAAAATTCCGAGGGCACAGGACAA
GCAGCAGATCTTAAAAGCACTCAAGCAGCCATCGACCAAATCAATGGGAAACTGAATAGGGTAATCGAGAAGACGA
ACGAGAAATTCCATCAAATCGAAAAGGAATTCTCAGAAGTAGAAGGGAGAATTCAGGACCTCGAGAAATACGTTGA
AGACACTAAAATAGATCTCTGGTCTTACAATGCGGAGCTTCTTGTCGCTCTGGAGAACCAACATACAATTGATCTG
ACTGACTCGGAAATGAACAAACTGTTTGAAAAAACAAGGAGGCAACTGAGGGAAAATGCTGAGGACATGGGCAATG
GTTGCTTCAAAATATACCACAAATGTGACAATGCTTGCATAGGGTCAATCAGAAATGGTACTTATGACCATGATGT
ATACAGAGACGAAGCATTAAACAACCGGTTTCAGATCAAAGGTGTTGAACTGAAGTCAGGATACAAAGACTGGATC
CTGTGGATTTCCTTTGCCATATCATGCTTTTTGCTTTGTGTTGTTTTGCTGGGGTTCATCATGTGGGCCTGCCAAA
AAGGCAACATTAGGTGCAACATTTGCATTTGA
Betainfluenzavirus virus (Influenza B/Florida/4/2006) hemagglutinin (GenBank: EU515992.1)
B/Yamagata lineage (SEQ ID NO: 8)
ATGAAGGCAATAATTGTACTACTCATGGTAGTAACATCCAATGCAGATCGAATCTGCACTGGAATAACATCTTCAA
ACTCACCTCATGTGGTCAAAACAGCCACTCAAGGGGAGGTCAATGTGACTGGTGTGATACCACTAACAACAACACC
AACAAAATCTTATTTTGCAAATCTCAAAGGAACAAGGACCAGAGGGAAACTATGCCCAGACTGTCTCAACTGCACA
GATCTGGATGTGGCTTTGGGCAGACCAATGTGTGTGGGGACCACACCTTCGGCGAAAGCTTCAATACTCCACGAAG
TCAAACCTGTTACATCCGGGTGCTTTCCTATAATGCACGACAGAACAAAAATCAGGCAACTACCCAATCTTCTCAG
AGGATATGAAAATATCAGGCTATCAACCCAAAACGTCATCGATGCGGAAAAGGCACCAGGAGGACCCTACAGACTT
GGAACCTCAGGATCTTGCCCTAACGCTACCAGTAAGAGCGGATTTTTCGCAACAATGGCTTGGGCTGTCCCAAAGG
ACAACAACAAAAATGCAACGAACCCACTAACAGTAGAAGTACCATACATTTGTACAGAAGGGGAAGACCAAATCAC
TGTTTGGGGGTTCCATTCAGATGACAAAACCCAAATGAAGAACCTCTATGGAGACTCAAATCCTCAAAAGTTCACC
TCATCTGCTAATGGAGTAACCACACACTATGTTTCTCAGATTGGCAGCTTCCCAGATCAAACAGAAGACGGAGGAC
TACCACAAAGCGGCAGGATTGTTGTTGATTACATGATGCAAAAACCTGGGAAAACAGGAACAATTGTCTACCAAAG
AGGTGTTTTGTTGCCTCAAAAGGTGTGGTGCGCGAGTGGCAGGAGCAAAGTAATAAAAGGGTCCTTGCCTTTAATT
GGTGAAGCAGATTGCCTTCATGAAAAATACGGTGGATTAAACAAAAGCAAGCCTTACTACACAGGAGAACATGCAA
AAGCCATAGGAAATTGCCCAATATGGGTGAAAACACCTTTGAAGCTTGCCAATGGAACCAAATATAGACCTCCTGC
AAAACTATTAAAGGAAAGGGGTTTCTTCGGAGCTATTGCTGGTTTCCTAGAAGGAGGATGGGAAGGAATGATTGCA
GGCTGGCACGGATACACATCTCACGGAGCACATGGAGTGGCAGTGGCGGCGGACCTTAAGAGTACGCAAGAAGCTA
TAAACAAGATAACAAAAAATCTCAATTCTTTGAGTGAGCTAGAAGTAAAGAATCTTCAAAGACTAAGTGGTGCCAT
GGATGAACTCCACAACGAAATACTCGAGCTGGATGAGAAAGTGGATGATCTCAGAGCTGACACTATAAGCTCGCAA
ATAGAACTTGCAGTCTTGCTTTCCAACGAAGGAATAATAAACAGTGAAGATGAGCATCTATTGGCACTTGAGAGAA
AACTAAAGAAAATGCTGGGTCCCTCTGCTGTAGAGATAGGAAATGGATGCTTCGAAACCAAACACAAGTGCAACCA
GACCTGCTTAGACAGGATAGCTGCTGGCACCTTTAATGCAGGAGAATTTTCTCTCCCCACTTTTGATTCACTGAAC
ATTACTGCTGCATCTTTAAATGATGATGGATTGGATAACCATACTATACTGCTCTATTACTCAACTGCTGCTTCTA
GTTTGGCTGTAACATTGATGCTAGCTATTTTTATTGTTTATATGGTCTCCAGAGACAACGTTTCATGCTCCATCTG
TCTATAA
Betainfluenzavirus (Influenza B/Victoria/2/87) hemagglutinin (GenBank: M58428.1) B/Victoria lineage
(SEQ ID NO: 9)
ATGAAGGCAATAATTGTACTACTCATGGTAGTAACATCCAATGCAGATCGAATCTGCACTGGGATAACATCGTCAA
ACTCACCCCATGTGGTCAAAACTGCTACTCAAGGGGAAGTCAATGTGACTGGTGTGATACCACTGACAACAACACC
AACCAAATCTCATTTTGCAAATCTCAAAGGAACAAAAACCAGAGGGAAACTATGCCCAAAGTGTCTCAACTGCACA
GATCTGGACGTGGCCTTGGCGAGACCAAAGTGCATGGGGACCATACCTTCGGCAAAAGCTTCAATACTCCATGAAG
TCAAACCTGTTACATCTGGGTGCTTTCCTATAATGCACGACAGAACAAAAATTAGACAGCTACCCAATCTTCTCAG
AGGATACGAACATATCAGGTTATCAACCCATAACGTTATCAACGCAGAAACGGCACCAGGAGGACCCTACAAAGTT
GGAACCTCAGGGTCTTGCCCTAACGTTACCAATGGAAACGGATTCTTCGCAACAATGGCTTGGGCTGTCCCAAAAA
ACGACAACAACAAAACAGCAACAAATCCATTAACAGTAGAAGTACCATACATTTGTACAGAAGGAGAAGACCAAAT
TACTGTTTGGGGGTTCCACTCTGATAGCGAAACCCAAATGGTAAAACTCTATGGAGACTCAAAGCCTCAGAAGTTC
ACCTCATCTGCCAATGGAGTAACCACACATTACGTTTCACAGATTGGTGGCTTCCCAAATCAAGCAGAAGACGGAG
GGCTACCACAAAGCGGTAGAATTGTTGTTGATTACATGGTGCAAAAATCTGGAAAAACAGGAACAATTACCTACCA
AAGAGGTATTTTATTGCCTCAAAAAGTGTGGTGCGCAAGTGGCAGGAGCAAGGTAATAAAAGGGTCCTTGCCTTTA
ATTGGTGAAGCAGATTGCCTCCACGAAAAATACGGTGGATTAAACAAAAGCAAGCCTTACTACACAGGAGAACATG
CAAAAGCCATAGGAAATTGCCCAATATGGGTGAAAACACCCTTGAAGCTGGCCAATGGAACCAAATATAGACCTCC
TGCAAAACTATTAAAGGAAAGGGGTTTCTTCGGAGCTATTGCTGGTTTCTTAGAAGGAGGATGGGAAGGAATGATT
GCAGGTTGGCACGGATACACATCCCATGGAGCACATGGAGTAGCAGTGGCAGCAGACCTTAAGAGTACGCAAGAAG
CCATAAACAAGATAACAAAAAATCTCAATTCTTTGAGTGAGCTGGAAGTAAAGAATCTTCAAAGACTAAGCGGTGC
CATGGATGAACTCCACAACGAAATACTCGAACTGGATGAGAAAGTGGATGATCTCAGAGCTGATACAATAAGCTCG
CAAATAGAGCTCGCAGTCTTGCTTTCCAACGAAGGAATAATAAACAGTGAAGATGAGCATCTCTTGGCGCTTGAAA
GAAAACTGAAGAAAATGCTGGGCCCCTCTGCTGTAGAGATAGGGAATGGATGCTTCGAAACCAAACACAAGTGCAA
CCAGACCTGCCTAGACAGAATAGCTGCTGGCACCTTTAATGCAGGAGAATTTTCTCTTCCCACCTTTGATTCACTA
AATATTACTGCTGCATCTTTAAATGATGATGGATTGGATAATCATACTATACTGCTTTACTACTCAACTGCTGCTT
CCAGTTTGGCTGTAACATTGATGATAGCTATCTTTATTGTTTATATGGTCTCCAGAGACAATGTTTCTTGCTCCAT
CTGTCTATAG

A simple MN system with immediate burst release properties will be fabricated for the dermal delivery of the mRNA. The formulation of the stabilized mRNA complex will be prepared with specific ratios of lipid vs. protamine vs. mRNA vs. excipients in a polyvinylpyrrolidone (PVP) solution. This solution will then be casted into a prefabricated polydimethylsiloxane (PDMS) mold and the solution will be dried under vacuum. The PDMS mold will help to form the MN shape from the dried formulation. The MNs will then be transferred onto a supporting poly-lactide (PLA) array, pre-coated with PVP to facilitate the skin insertion and the dermal embedment. After the skin insertion, the sacrificial PVP layer will be quickly dissolved by the body fluid to release the MNs into the dermal layer of animals.

The loaded mRNA dose can easily be varied by changing the concentration of the mRNA particles formulated inside the precursor buffer for the mold-casting process to create the MNs. To quantify the amount of loaded mRNA, the mRNA MNs will be reconstituted by placing them into a PBS buffer and using UV-vis absorbance to detect the presence of mRNA. As the mixture will also include the protein and lipid, reported methods for light-scattering correction will be used to determine the specific peak of mRNA at the wavelength of 260/280 nm. We will further confirm the mRNA result, using a commercial RNA quantification kit (Quant-iT™ RiboGreen™, ThermoFisher).

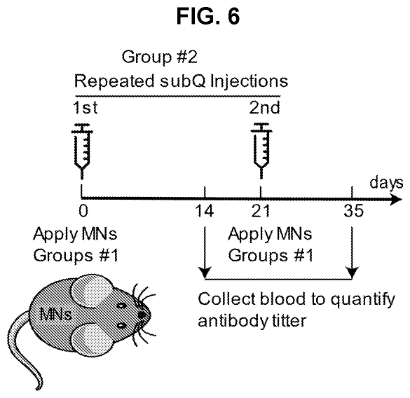

To study the immunogenicity of the stabilized mRNA via the dermal MN delivery after heat exposure, the MNs can also be exposed to heat by incubation at 37° C. for 1 month in vitro after fabrication. This time period mimics the time of embedding a final MN version (with built-in burst releases at days 0 and 21) inside the skin. After the heat exposure, the MNs will be administered into rats at the same time points of the subcutaneous injections (i.e., days 0 and 21), as shown in FIG. 6.

Sprague Dawley rats (n=6 rats/group) will be employed instead of mice as rats have a sufficiently thick dermal layer (about 400-600 μm), while the dermal layer of mice is only about 100 μm thick. In comparison, the dermal layer of humans is about 2000 μm thick. Therefore, the thicker dermal layer of rats better simulates the skin layer of humans and will enable the MNs to fully target the dermal area. One animal group will receive the dermal MNs at day 0 and day 21 with an mRNA dose of 80 μg per each MN release. The other animal group will receive two subcutaneous injections of the same mRNA dose at day 0 and day 21 (80 μg/injection). The specific dose of 80 μg/injection has been previously reported for the rabies mRNA vaccine in rodents. Only one vaccine shot (i.e., primer shot only) could be performed and then compared with the immunogenic effect with the MNs. However, to assure that a significant neutralizing antibody build-up is obtained against the stabilized mRNA vaccines for ELISA detection, a booster shot at day 21 will also be used. The blood will then be collected at day 14 and day 35 (14 days after the shot/MN-release) to quantify the antibody titer via the use of ELISA assay, as shown in FIG. 6. To collect the blood, the animals will be temporarily restrained using restrainers. Up to 100 uL of blood will be collected from the lateral tail vein or lateral saphenous vein of the animals at one time.

Throughout the study, animals will be monitored daily by observing the skin for any irritation, as well as monitored for any other health signals including weight, signs of pain, etc. The rats will be monitored for signs of distress and weighed daily. If a rat exhibits a high clinical score or exceeds 30% weight loss, it will be humanely euthanized following established methods.

### Example 8

### Fabrication and Assessment of MNs for Immediate Release

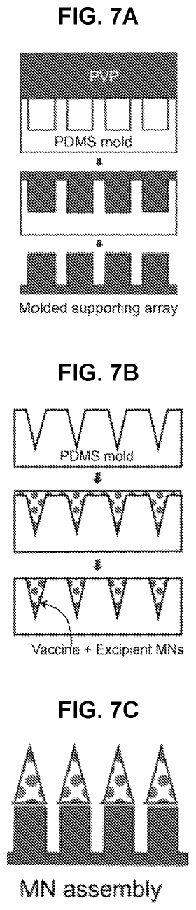

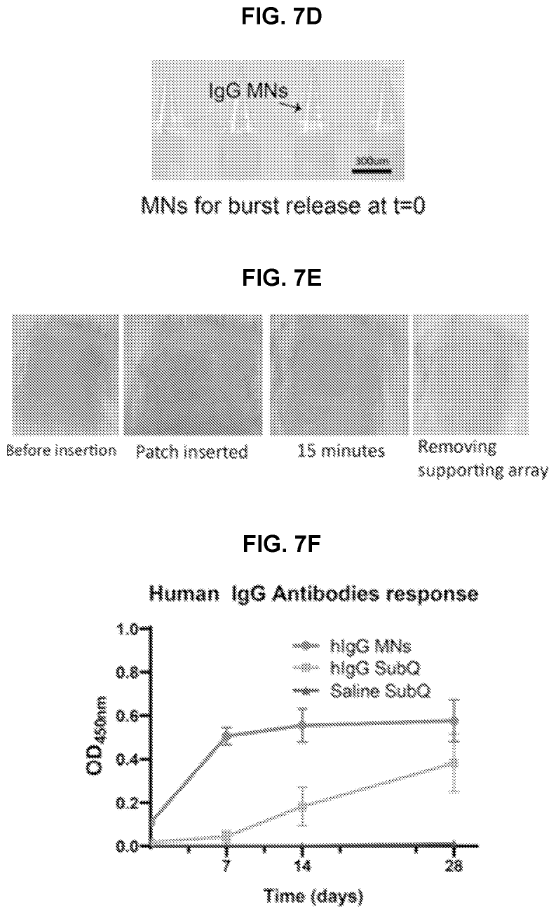

The MNs were produced by making the PLGA shell and the vaccine MN core separately prior to assembling them together. As such, for the burst release at time 0, the vaccine MN core without the PLGA shell can be utilized. The molding process (illustrated in FIG. 7A-C) is performed with the drying process, which is used to formulate the stabilized mRNA of earlier Examples. The mRNA-protamine complex in LNPs mixed with excipients may be casted into a PDMS mold and dried under vacuum which draws the mixture into the empty wells of the mold and will dry it at the same time. The vaccine MNs can then be assembled onto a supporting array patch for skin insertion (FIG. 7C).

The immediate burst release has been demonstrated from this simple vaccine MN system where the MNs were embedded inside rats to obtain the immediate burst release of a vaccine model (IgG) at time 0 (FIG. 7D-F).

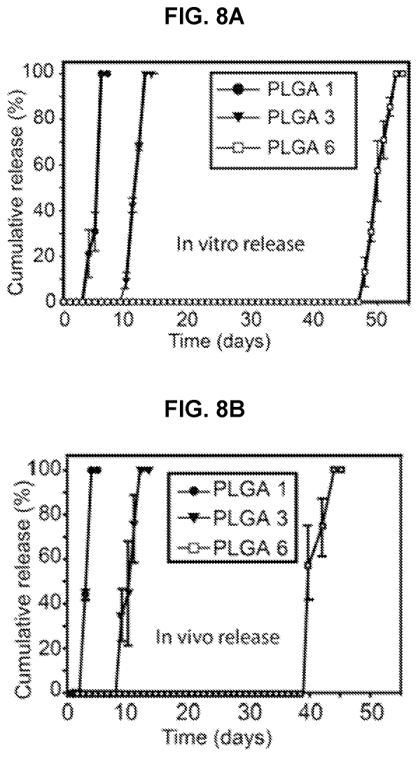

Furthermore, it has been shown that the release kinetics of formulated MNs with different PLGA shells are very similar between in vivo and in vitro conditions, using a buffer of PBS at 37° C. (FIG. 8A-B). As such, a good in vitro condition has already been established to study the in vivo dissolution of the MN formulations.

### Example 9

### Different MN Shells Can Obtain Different Release Bursts

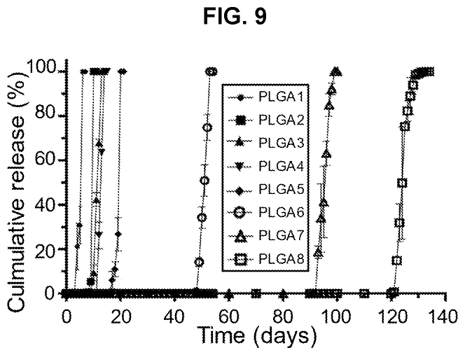

To generate core-shell MNs for delayed burst release, the fabrication method previously described in US 2019/026989 5A1 was utilized, which is incorporated by reference herein for such teachings. This described fabrication method is related to loading an mRNA MN core into a PLGA shell. Different shells may also be used to obtain different release bursts at various time points (FIG. 8A-B). FIG. 9 also shows a graph of the cumulative release of mRNA from MNs having different PLGA shells (PLGA1-8) with varying delayed release properties.

### Example 10

### Amino Acids Can Further Enhance mRNA Stability

Besides the protection by sugar excipients such as trehalose and sucrose, the addition of certain amounts of amino acids (e.g., 0.3% lysine) before air or vacuum drying can further enhance the stability of mRNA-protamine-liposome formulations during drying and against heat conditions. These formulations were prepared by mixing mRNA with protamine at RT and incubating at RT for 15 min. The liposomes were then added to the formulation and incubated at RT for 30 min. Lysine was then added to the solution, followed by trehalose, and the formulation was mixed.

Once the formulations were made, in-tube studies for rapid screening were performed. The formulations were aspirated to 1.5 mL tubes and dried under vacuum overnight. A portion of tubes with dried formulations and freshly made formulations were heated to 80° C. for 2 hr, while the rest of the tube samples with either dried formulations or freshly made formulations were kept at RT for 2 hr. Water was added to reconstitute the dried formulations and then HeLa cells were treated in 384 well plates with the resultant formulations (RT and 80° C.) and freshly made formulations (RT and 80° C.). After 24 hr, bioluminescence signal was measured.

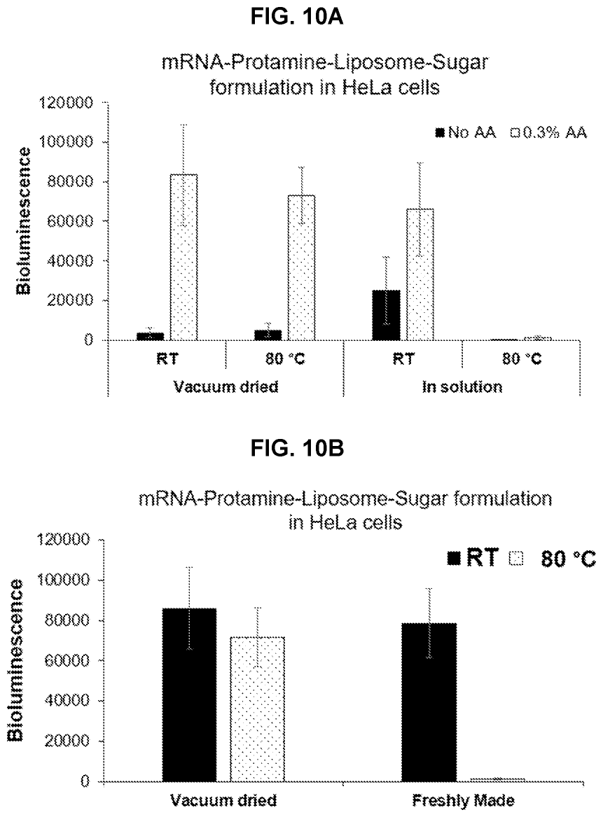

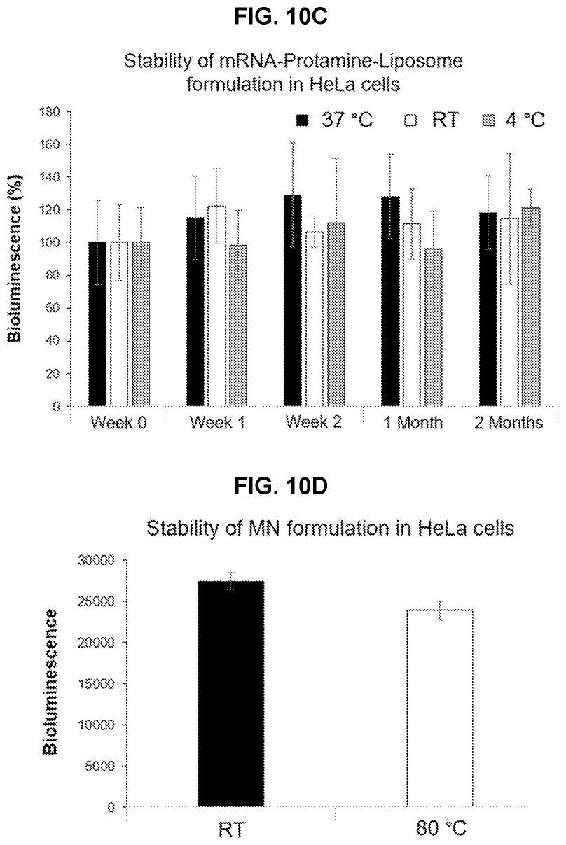

In-tube studies to test long-term stability were also performed. The formulations were aspirated to 1.5 mL tubes and dried under vacuum overnight. The dried formulations and freshly made formulations were stored at 4° C., RT, and 37° C. for different time periods (1-week, 2-weeks, 1-month, and 2-months). At each time point, water was added to reconstitute the dried formulations in tubes and the cells were then treated in 384 well plates with the resultant formulations and freshly made formulations. After 24 hr, bioluminescence signal was measured. As shown in FIG. 10A-C, a final formulation of mRNA+protamine+liposome (DC-Chol:DOPE=1:1)+sugar (trehalose)+amino acid (lysine) exhibited improved stability after vacuum drying and heat exposure for certain periods of time (either 80° C. for 2 hr or 37° C. for up to 2 months) when transfected in HeLa cells. FIG. 10B shows that vacuum dried formulations of mRNA can achieve comparable transfection efficiency as freshly made formulations. FIG. 10C demonstrates that bioluminescence signal was not lost at 2 months compared to week 0.

Promising stability data is also shown in FIG. 10D, where after the formulations were loaded into MNs, the MNs were exposed to 80° C. for 2 hr, similar to what was done in tubes, and then the transfection efficiency was tested in HeLa cells. For these MN studies, the formulations were loaded onto a PDMS mold and vacuumed for 5 min to exclude bubbles in the tip, then the formulation was dried in a desiccator overnight. The scum layer was removed with water and the molds were dried under vacuum for 24 hr. PLGA was used to peel the MNs out of the mold and was then heated to 80° C. Water was added to reconstitute the dried formulations in tubes to dissolve the MNs and HeLa cells were then treated in 384 well plates with the resultant formulations. After 24 hr, bioluminescence signal was measured.

### Example 11

### A Single-Administration MN Skin Patch for Multi-Burst Release of Vaccine Against SARS-COV-2

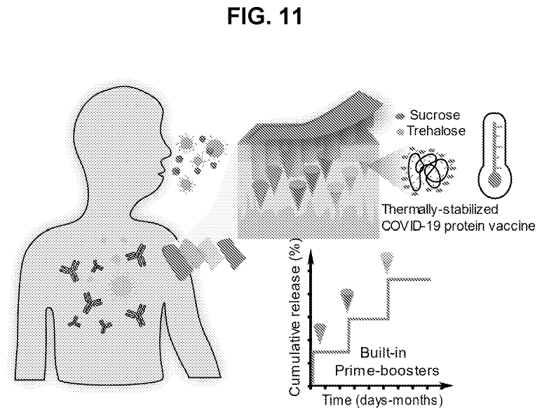

Vaccine access is inconsistent worldwide. Necessity for multiple injections and cold-chain storage have contributed to suboptimal vaccine utilization, especially in pandemic situations. Thermally stable and single-administration vaccines hold a great potential to revolutionize the global immunization process. Presented herein is a new approach to thermally stabilize protein-based antigens and devise a new high-throughput antigen-loading process to create a single-administration, pulsatile-release MN patch which can deliver a recombinant SARS-CoV-2 S1-RBD vaccine for the COVID-19 pandemic (FIG. 11). Nearly 100% of the protein antigen could be stabilized at temperatures up to 100° C. for at least 1 hr and at an average human body temperature (37° C.) for up to 4 months. Arrays of the stabilized S1-RBD formulations can be loaded into arrays of the MN shells via a single-alignment assembly step. The fabricated MNs were administered at a single time into the skin of rats and induced an antibody response which could neutralize authentic SARS-CoV-2 viruses, providing similar immunogenic effect to that induced by multiple bolus injections of the same antigen stored in conventional cold-chain conditions. The MN system presented herein could offer the key solution to global immunization campaigns by avoiding the low patient compliance, the requirement for cold-chain storage, and the need of multiple booster injections.

### Fabrication of the Core-Shell Microneedles (MNs)

The MN patch manufacturing protocol follows previously reported methods of micro-molding and 3D layer-by-layer assembly with some additional modifications. The four main components of the core-shell MN are a shell, a core, a cap, and a supporting array. The shell and the cap were made by compression-molding a Poly(D,L-lactide-co-glycolide) (PLGA) (Millipore Sigma and Polysciences, Inc., USA) film into respective PDMS molds in the vacuum oven for 20 minutes at PLGA glass transition temperature. A Carver press (Model: 3850-1011, Wabash, Ind.) was used to make PLGA films with define thickness. The “scum layer” (or residual film) resulted from the molding process was then removed by gently dropping and wiping acetone on top of the mold. The vaccine-core preparation included dissolving or suspending vaccine antigen S1-RBD protein in excipient solutions 0.5 M Trehalose (Millipore Sigma and Sucrose (Millipore Sigma) dissolved in MilliQ water. In the next step, the solution or suspension was filled into the prepared inverse PDMS mold. After solvent evaporation, the scum layer was then removed. The scum-free vaccine-cores were next transferred onto a sacrificial PLGA film under mild heating. This whole structure was delaminated onto a solid Teflon-coated substrate via heat-assisted micro-transfer molding. The supporting array was made by a two-step process involving solution casting effervescent polymer solution and PLA molding to provide a flexible backing layer.

### Drug Loading Process and 3D Layer-by-Layer Assembly

The core-shell MNs assembly process, which was based on previously reported technology named StampEd Assembly of polymer Layers (SEAL), combines the technology used for computer chip manufacturing with soft lithography and an aligned sintering process. First, the core-drugs previously prepared were aligned using the aligning device and loaded into the core-shell MNs. Secondly, the scum layer was them removed by the method described above, using acetone and water as solvents. The caps inside the PDMS mold are aligned and loaded on top of the PLA-effervescent supporting array and sintered using heat. In the final step, the core-shell MNs were transferred onto the supporting array. The mold entrapping the core-shell MNs was then peeled off to yield free-standing core-shell MNs on the supporting array which could be fully embedded into the skin. The optical images of the microneedles were taken by the AmScope ME300TZC-2L-8M with a digital camera (AmScope Mu800).

### S1-RBD Protein Stability Study

S1-RBD-protein (Euprotein Inc., North Brunswick, N.J.) was mixed with Trehalose-Sucrose (TS) solution at different ratios (shown below in Table 3) and vacuum dried overnight (mimicking the MN core fabrication). Samples were then exposed to heat for short-term stability screenings (1 hour) at 56° C., 70° C., and 100° C. Long-term stability study was conducted at 37° C. (body temperature) for 2 weeks, 4 weeks, 8 weeks, 12 weeks, and 16 weeks.

TABLE 3
Excipient formulations to stabilize S1-RBD protein

| | | F2 | F3 | F4 |
|---|---|---|---|---|
| Compound | F1 | (1:1 v/v) | (1:2 v/v) | (1:4 v/v) |
| Recombinant protein of the receptor binding | 2.5 μL | 2.5 μL | 2.5 μL | 2.5 μL |
| domain of 2019-nCov Spike S1, with a C-terminal | | | | |
| polyhistidine tag (2 mg/mL - Euprotein | | | | |
| (EPB226512) | | | | |
| 0.5M (171 mg/mL) Trehalose + Sucrose (0.5 | 0 μL | 2.5 μL | 5 μL | 7.5 μL |
| MTS) | | | | |

ELISA was used to determine protein activity after heat exposure. Incubated and non-incubated S1-RBD-protein were diluted to a final concentration of 5 μg/mL in 100 mM carbonate-bicarbonate buffer (pH 9.6). BSA was diluted in carbonate-bicarbonate buffer to an equivalent concentration and used as a negative control. Immulon1B plates (Thermo Fisher Scientific) were coated with 100 μL per well with S1-RBD in bicarbonate buffer. Plates were then washed with 0.05% Tween-20 in PBS (Thermo Fisher Scientific) and blocked for 1 hour at room temperature with 2 μg/mL bovine serum albumin (BSA) 0.05% Tween-20 in PBS. As a primary antibody, 100 μL of SARS-CoV-2 Spike Protein (RBD) Recombinant Human Monoclonal Antibody (Thermo Fisher Scientific) diluted at 1:500 in PBST was loaded into each well and incubated for 1 hr at room temperature. Wells were then washed three times with 50 μL of PBST before loading the secondary antibodies. Goat anti-Human IgG (Gamma chain) Cross-Adsorbed Secondary Antibody, HRP diluted at 1:1,000 in PBST (Thermo Fisher Scientific) was used as a secondary antibody (100 μL) and the samples were incubated for 1 hr at room temperature. All of the wells were then washed three times with 50 μL of PBST before development. To develop each plate, 100 μL TMB substrate (SouthernBiotech) was added to each well. After 15 min, the reaction was stopped with TMB Stop Solution (SouthernBiotech). Plates were read at 450 nm on a BioTek Synergy HT plate reader using BioTek Gen5 (BioTek). Data are representative of an average of three sample replicates and normalized to initial S1-RBD-protein activity after vacuum drying.

SDS-Page Coomassie Blue-stained SDS-PAGE gel of S1-RBD under varying conditions. Lane L—Ladder, Lane 1—Negative Control, Lane 2—S1-RBD stock, Lane 3—S1-RBD (dried), Lane 4—S1-RBD 80° C. (dried), Lane 5—S1-RBD+TS (dried), Lane 6—S1-RBD+TS 80° C. (dried). All dried samples were provided as 20 μg dried protein and were resuspended in 20 μL Ultrapure Dnase and Rnase free water. 10 μL of each sample was mixed 1:1 with Laemmli+BME, incubated at 70° C. for 10 min, and then 20 μL of each sample was loaded per well, and run for 20 min at 200 V. Gel was Coomassie Blue stained over the weekend, destained using destain buffer with methanol, and imaged on a GelDoc light imager.

### S1-RBD Protein Vaccination

For the dosing study, female SAS SD rats (aged 8 weeks; Charles River Laboratories) were immunized with S1-RBD-protein at 10 μg/dose, 20 μg/dose, 50 μg/dose, and 100 μg/dose via subcutaneous injections on days 0, 10, and 40. Saline was given as a negative control group. Blood collections were performed on days 0, 9, 14, 30, 44, 55, and 70. The serum was then collected from whole blood by centrifugation at 2,000 RPM for 5 min and stored at −20° C. until use.

The MN patch was applied using a commercially available applicator (Micropoint Technologies Pte Ltd, Singapore) for 15 minutes. Then, the supporting array was removed leaving the core-shell MNs embedded inside the skin. The subcutaneous injections were administered on days matching the MNs releases. Empty MNs were also given as a negative control group. Blood collections were performed accordingly. The serum was then collected from whole blood by centrifugation at 2000 RPM for 5 mins and stored at −20° C.

ELISA were performed on collected serum to determine the total binding antibody response. S1-RBD-protein (100 μL of 5 μg/mL) in 100 mM carbonate-bicarbonate buffer (pH 9.6) was added to Immulon 1B 96-well plates and incubated overnight at 4° C. Plates were then washed with 0.05% Tween-20 in PBS and blocked for 1 h at room temperature with 2 μg/mL BSA 0.05% Tween-20 in PBS. Plates were then washed three times with 0.05% Tween-20 in PBS. Serum diluted 1:10 in 0.05% Tween-20 in PBS were then added at 90 μL per well and incubated for 1 hr at room temperature. Wells were washed three times, and 100 μL of monoclonal goat anti-rat IgG horseradish peroxidase (HRP)-conjugated antibodies (1:1,000 in 0.05% Tween-20 in PBS) was added. After incubation for 1 hr at room temperature, wells were washed three times and 100 μL of TMB substrate solution was added to each well. The reaction was then stopped after 15 min by adding 100 μL TMB Stop Solution. Absorbance at 450 nm was measured using a Cytation 5 microplate reader (BioTek). The value was reported as optical density at 450 nm. For end-point binding titer, two-fold serial dilutions were conducted. End-point titers were determined as the final dilution at which signal in a well exceeded 3 times the background signal of the assay (as determined by averaging negative control values). Endpoint titers were reported as the reciprocal of that final dilution. Statistical analysis was performed in GraphPad Prism 8.0 (GraphPad).

### Plaque Reduction Neutralization Test (PRNT)

Vero-E6 cells (ATCC) were seeded the day before the experiment in 48-well plates at 40,000 cells per well in 250 μL of D10+ media (DMEM (Corning) supplemented with HEPES (Corning), 1× Penicillin 100 IU/mL/Streptomycin 100 mg/mL (Corning), 1× Glutamine (Glutamax, ThermoFisher Scientific), and 10% Fetal Bovine serum (FBS) (Sigma)) following harvest with Trypsin-EDTA (Fisher Scientific). The serum samples were thawed on ice and diluted in D+ media (DMEM supplemented with HEPES, 1× Penicillin 100 IU/mL/Streptomycin 100 mg/mL and 1× Glutamine) to the desired concentrations in 96-well dilution blocks (Corning). Diluted stocks of SARS-CoV-2 variants (ancestral US-WA1/2020 at 5×10⁻⁵ or delta B.1.617.2 at 5×10⁻⁴) were prepared in D+. An aliquot of viral stock was added to the serum dilution wells and the negative control wells (D+ only) in the dilution block to achieve the desired final concentration (5×10⁻⁶ for US-WA1/2020 and 5×10⁻⁵ for delta) and incubated for 1 h at 37° C. at 5% CO₂. For each serum sample, 50 uL of virus-serum dilutions (or no serum and no virus no serum controls) were added to triplicate wells in 48-well plate. The viral inoculum was removed after 1 hour and 0.5 mL of 1% methylcellulose (VWR) in DMEM supplemented with HEPES, 1× Penicillin 100 IU/mL/Streptomycin 100 mg/mL, 1× Glutamine and 2% FBS was added to each well. After a 6-day incubation at 37° C. and 5% CO₂, the methylcellulose media was carefully removed from each well, the cells were fixed in ice-cold methanol at −20° C. for 15 min and the plaques revealed with 1% aqueous Crystal violet solution (Fisher) and subsequent washes with water. The number of plaques in each well was counted manually.

The US-WA1/2020 ancestral variant was obtained from BEI Resources. The delta variant isolate was obtained from the MassCPR variant repository. In brief, the variant was isolated at the Ragon BSL3 by rescue on Vero-E6 cells from primary clinical specimens. The whole genome of subsequent viral stocks was sequenced to confirm that no additional mutation arose during virus expansion. Statistical analysis was performed in GraphPad Prism 8.0 (GraphPad).

### Mechanical Testing

An ADM ET eXpert 5952F fatigue tester (ADMET, USA) was equipped with two machined aluminum grips in its upper and lower clamp. MN patches were adhered to the lower aluminum grips with a thin layer of Loctite 411 adhesive (Henkel, Germany). The upper piston was lowered to apply compressive force to the MN patch at a rate of 6 mm/min and data was sampled at 100 samples/second. The compressive force was triggered to stop at a ceiling value of 444.8 N. The compression testing data was plotted as compression force per needle as a function of the displacement.

### Skin irritation Assessment

The optical images of the rats' skin were taken right after the MNs insertion and throughout the vaccination study. Three individuals who were blinded to the experiment setup scored the severity of erythema, eschar, and edema. Four (4) is the maximum possible score which indicates the severe skin irritation and (0) is the minimum score for normal, no irritation skin. The scores were displayed as the average and standard error of the mean (SEM).

### Stabilization of S1-RBD-Protein Antigen for Long-Term Skin-Embedded MNs

The need to overcome the cold-chain storage requirements and maintain vaccine thermostability is a major barrier to most vaccination campaigns. Incorporating vaccines into MN patches and eliminating the need for a reconstitution process can enable an enhanced thermostability as compared to liquid form. Still, developing stabilized formulations that are specific to each vaccine and MN manufacturing process is essential for patch production, storage, and usage. Throughout the process of drying and storage, proteins can undergo critical stresses owing to the removal of their hydration shell which makes them susceptible to mis-folding and denaturation. Combinations of excipients which could successfully stabilize a protein antigen derived from the S1 protein of SARS-CoV-2 and containing the Receptor Binding Domain (RBD) of the virus were sought to be identified for implementing the single-administration MN platform. Trehalose-Sucrose (TS) was selected as the stabilizing excipients (FIG. 11). This was because Trehalose and Sucrose could serve as water substitutions by direct interactions with proteins through hydrogen bonding. The sugars additionally help to minimize protein exposure to the environment and fill in void volumes. Highly viscous trehalose/sugar glass also entraps protein conformations, slows molecular dynamics, and eventually prevents protein misfolding. In this experiment, formulation screenings were performed for S1-RBD protein antigen to identify the best formulation that would properly stabilize the antigen in varying temperatures. The S1-RBD protein antigen was mixed with 0.5 M TS solution at 3 different ratios of 1:1 v/v, 1:2 v/v, and 1:4 v/v (Table 3) and then vacuum dried overnight with desiccant at room temperature.

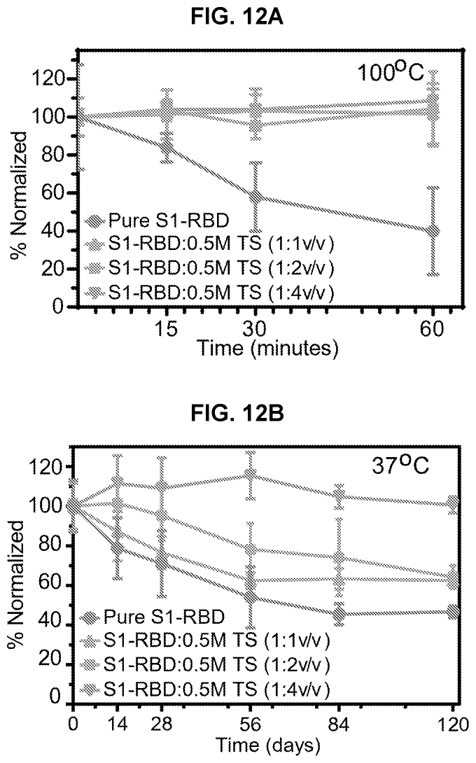

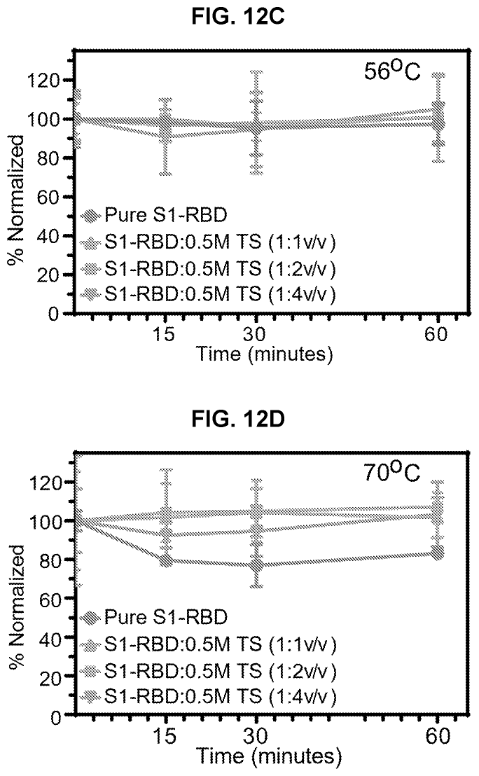

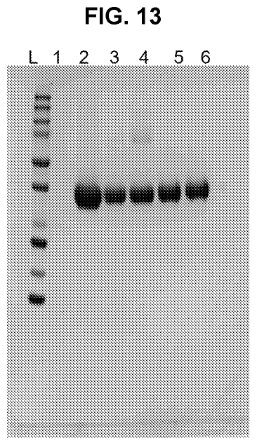

Samples were exposed to heat for short-term stability screenings (1 hr) at 56° C., 70° C., and 100° C. to mimic temperatures that may be observed during the MN manufacturing process. A long-term stability analysis was conducted at 37° C. (approximate human body temperature) for 2 weeks, 4 weeks, 8 weeks, 12 weeks, and 16 weeks. ELISAs were used to assess antigenicity of S1-RBD protein after heat exposure. FIG. 12A-D illustrate the stabilization of S1-RBD protein using the various TS formulations. While the pure S1-RBD is quite stable at 56° C. for a short period of time (˜60 mins), the protein quickly degrades at 75-100° C. and exhibits a significant degradation for a long-term heat exposure at 37° C. In contrast, formulations of S1-RBD which were mixed with TS retained 100% S1-RBD protein activity even at high temperatures of 75-100° C. after 1-hr incubation. SDS-PAGE analysis further confirmed that the S1-RBD protein formulated with TS maintained its structural integrity after heat exposure (FIG. 13). Moreover, long-term incubation at 37° C. indicated that the formulations with greater volumes of TS provided improved protein stability and outperformed those with lower concentrations of TS. Specifically, the S1-RBD:TS at 1:4 v/v ratio formulation retained 100% antigen activity. The 1:1 v/v and 1:2 v/v S1-RBD:TS ratios maintained 70% and 80% activities, respectively, in long-term stability study. In comparison, pure S1-RBD-protein without excipients demonstrated a 60% reduction in activity. In general, the presence of TS as excipients has provided a crucial protection to S1-RBD at high temperatures for both short-term and long-term incubation intervals. To sum up, suitable excipient formulations have been identified using various TS ratios to stabilize S1-RBD for short-term (up to 1 hr) under high heat exposure (75-100° C.) and long-term (up to 16 weeks) at average human body temperature of 37° C., which is important for the step of loading the protein into MNs.

### New MN Fabrication Process with a High-Throughput, One-Step Loading ff Stabilized S1-RBD Antigen into MN Patch

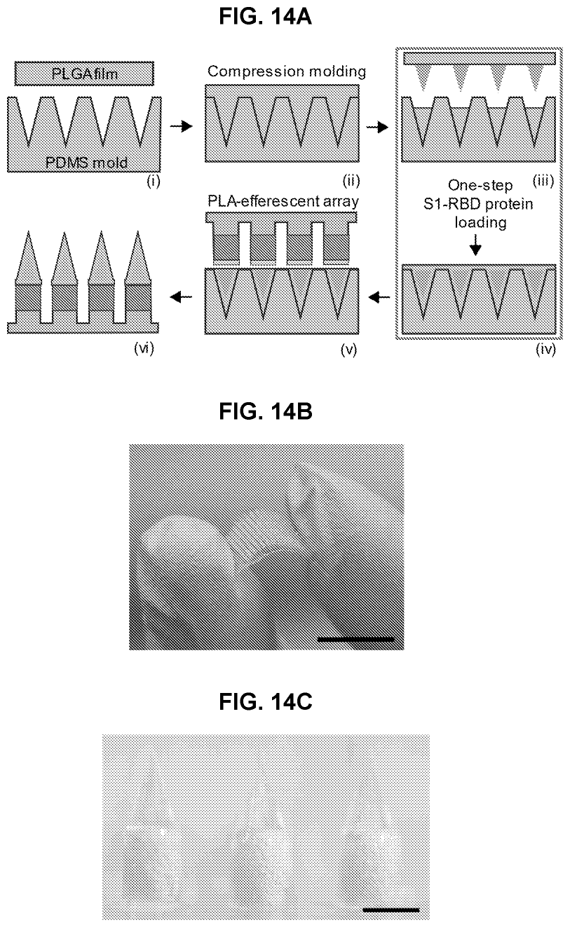

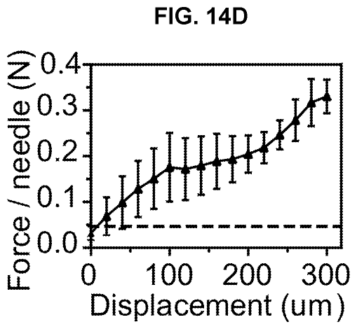

The MN patch manufacturing, presented herein, is technically based on previously reported work and yet, a new vaccine-core loading process was developed to further improve the manufacturing throughput and avoid multiple complex layer-by-layer alignment steps which are prone to errors and potentially cause loss of vaccine antigens. Using micro-molding, 3D assembling, and heat sintering processes, a core-shell MN structure was created with three main components including a polymeric shell, a cap, and a dried vaccine core, positioned on top of a dissolvable supporting structure (FIG. 14A-D). Each MN has a height of 600 μm and a base diameter of 300 μm and encapsulates a vaccine-loaded core with a height of 400 μm and a diameter of 200 μm. Briefly, a biodegradable polymer film of PLGA is compression-molded into a silicone or PDMS mold to form the base for the MN shell. The excessive PLGA scum layer on top of PDMS mold is then removed (FIG. 14A). The S1-RBD vaccine cores are made separately by casting S1-RBD protein mixed in TS solutions (1:4 v/v ration) onto another PDMS mold, which has structure and relative spacing similar to the MN shell mold. After a drying period, the arrays of the micro-molded vaccine cores are then transferred onto a PLGA film. The key novelty of this fabrication process is that the arrays of the vaccine-cores are aligned and are loaded directly into arrays of the heated MN bases/shells previously prepared (FIG. 14A). The whole MN part is then placed in a heated vacuum oven for a short period of time, which allows the PLGA polymer to fill in the PDMS mold and encapsulate the loaded vaccine cores.

To further elaborate on the novelty of the antigen-loading process, only one alignment is performed to load arrays of the vaccine-cores into the MN shells, while previous work required two separate alignment steps to (1) impinge the PLGA shell for creation of empty wells, and (2) insert the MN core into the wells. Thus, the fabrication method herein significantly enhances the throughput and reduces the complexity and potential errors caused by multiple alignment steps. This one-step loading procedure also offers a high consistency, efficiency, and maximizes the amount of loaded vaccine. In the next step, the core-shell MNs are capped and transferred onto a two-layered Polylactic acid (PLA)-effervescent supporting array (FIG. 14A). The effervescent layer provides a rapid detachment of the MNs from the supporting array upon contact with the local bodily fluid at the site of insertion, which minimizes the patch wear time (only 5-10 mins before patch removal) and optimizes the MN application process. The MNs are released from the PDMS mold to yield the final complete vaccine-loaded core-shell MN patch (FIG. 14A-C).

From previous work, the MNs have demonstrated their capabilities to penetrate and stay embedded under the skin (like an invisible tattoo) following insertion and rapid healing of the skin. The patch can be safely removed and disposed after administration (about 5-10 mins) and produces no biohazardous sharp wastes.

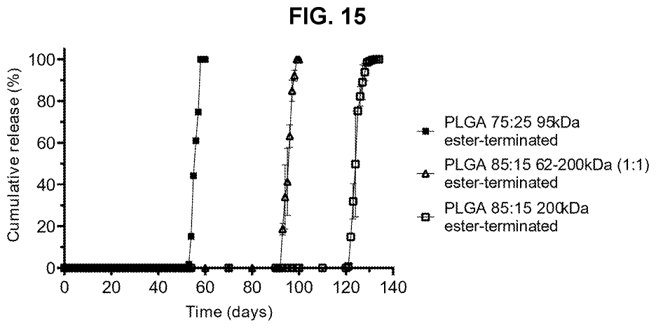

Mechanical strength measurement (FIG. 14D) indicated a failure force of 0.15 N per MN (under compression), which is sufficient for MNs penetration into the human skin without damages to the MN structures. The release times of the MNs are highly tunable by tailoring the molecular weight and compositions of the PLGA shell to accommodate different antigen release paradigms. Using PLGA shells with high molecular weights and more lactide component, a delayed burst release (to mimic the booster dose) could be obtained up to 4 months, as shown in FIG. 15. In principle, multiple sets of MNs made of different PLGA shells could be combined into one patch for a single-time administration of antigen with built-in booster doses. The embedded MNs can provide multiple burst releases of the encapsulated payload after predictable periods of time, offering an effect similar to that obtained from conventional multiple bolus injections.

### In Vivo Immunogenicity of S1-RBD Protein-Loaded Core-Shell MNs

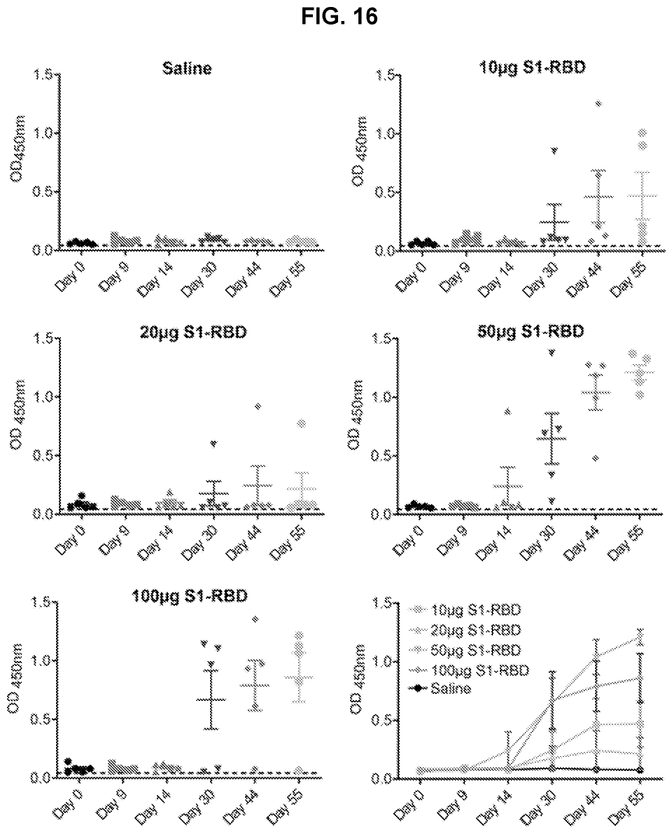

Prior to the MN construction, an appropriate dose of S1-RBD for use in the platform first needed to be identified. A dose-finding study was conducted by vaccinating SAS SD rats with the protein antigen at 10 μg/dose, 20 μg/dose, 50 μg/dose, and 100 μg/dose via subcutaneous injections on days 0, 10, and 40. Serum from vaccinated rats was collected and analyzed using ELISA to track changes in S1-RBD-specific IgG antibody responses over the course of the vaccination schedule (FIG. 16). The negative-control group that received saline injections on the same schedule did not demonstrate a detectable S1-RBD-specific antibody response. Animals receiving the 10 μg/dose and 20 μg/dose developed antibodies specific to S1-RBD protein though these responses were not statistically significantly higher than negative control animals. In contrast, the 50 μg/dose and 100 μg/dose groups exhibited a statistically significant S1-RBD-specific IgG responses at day 55 in comparison to the saline control animals. As there was no statistically significant increase in antigen-specific IgG induced by the 100 μg dose over the 50 μg dose, the 50 μg dose was selected for continuation of the study.

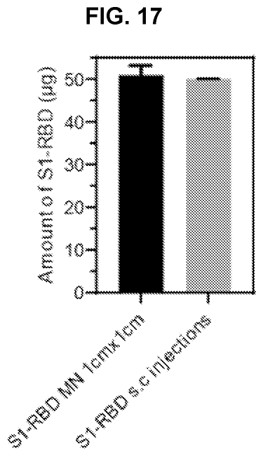

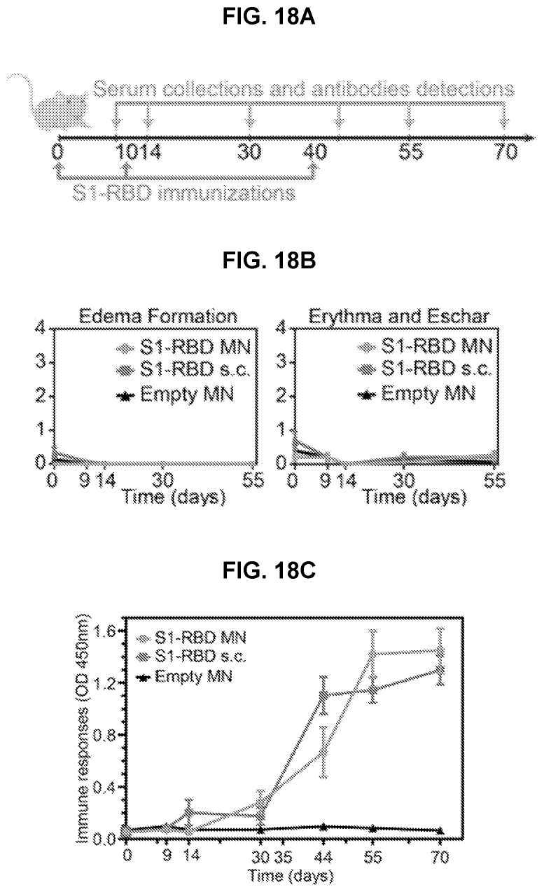

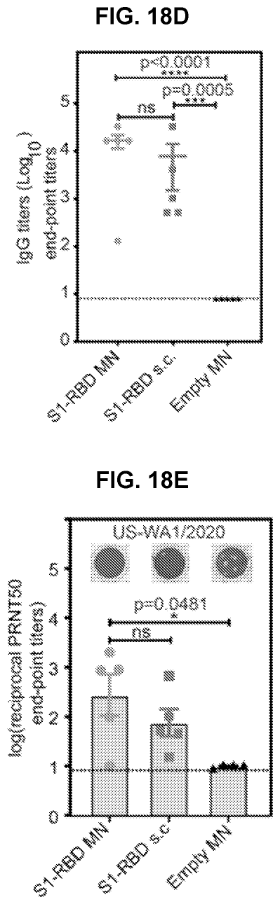

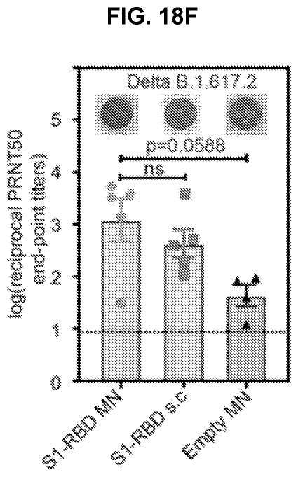

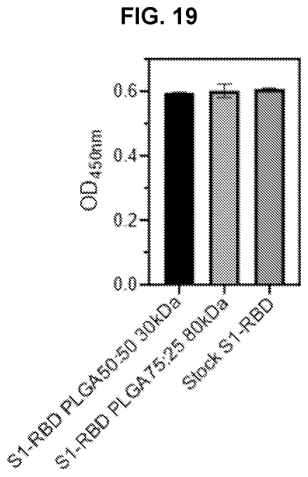

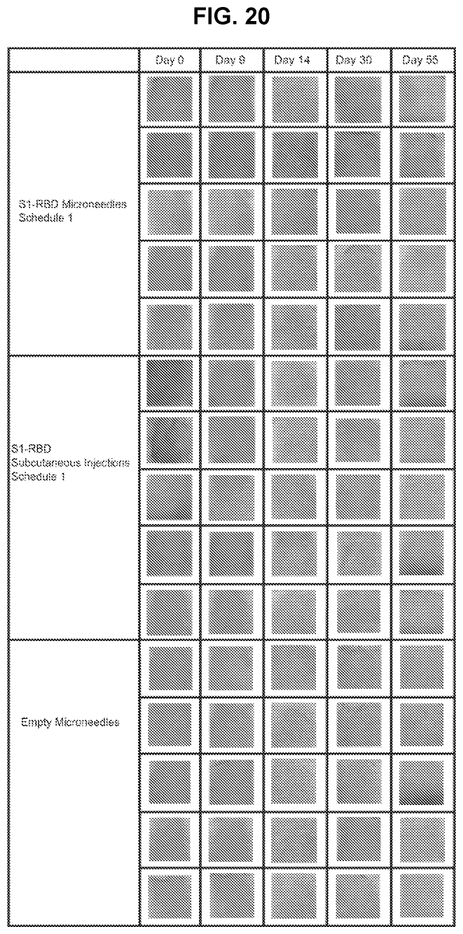

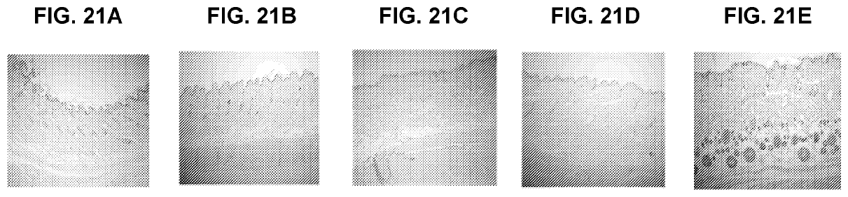

To demonstrate the immunogenicity of the S1-RBD loaded single-administration core-shell MNs, similar to the multiple vaccine-injection regime, the MN patches containing 50 μg S1-RBD protein were first fabricated and applied on rats. One 1 cm×1 cm MN patch could contain the same amount of active protein as used in subcutaneous injection. This dose matching and the stability of the S1-RBD protein after the MN fabrication process was confirmed using ELISA (FIG. 17). The rats then received S1-RBD vaccinations ata single time with the MN administration onto the skins (S1-RBD MN) or multiple-time subcutaneous bolus injections (S1-RBD s.c.). Subcutaneous, intradermal, and intramuscular routes have been shown to elicit similar immune responses in several vaccines and therefore, subcutaneous injections were chosen as a reference (similar to conventional vaccine injections in clinics) for comparison with MNs. A vaccination schedule was used that had a priming dosage started on Day 0 followed by two boosts on Day 10 and on Day 40 (FIG. 18A). The MNs were constructed accordingly (with the PLGA shells, adapted from previous work for the burst releases at days 0, 10, and 40) to replicate this administration schedule. Since PLGA degradation releases acidic byproducts that decrease the local environmental pH, the stability of R1-RBD was also assessed to ensure the protein maintained its stability at low pH (FIG. 19). The implantation of MNs inside the skin did not cause any noticeable skin erythema, edema, or any irritation and inflammation in all groups for the entire duration of the studies, which suggests that the core-shell MNs are safe and well-tolerated (FIG. 18B and FIG. 20). Histological analysis of the rat skin further showed no detectable traces of tissue damages or inflammatory cells (FIG. 21A-E).

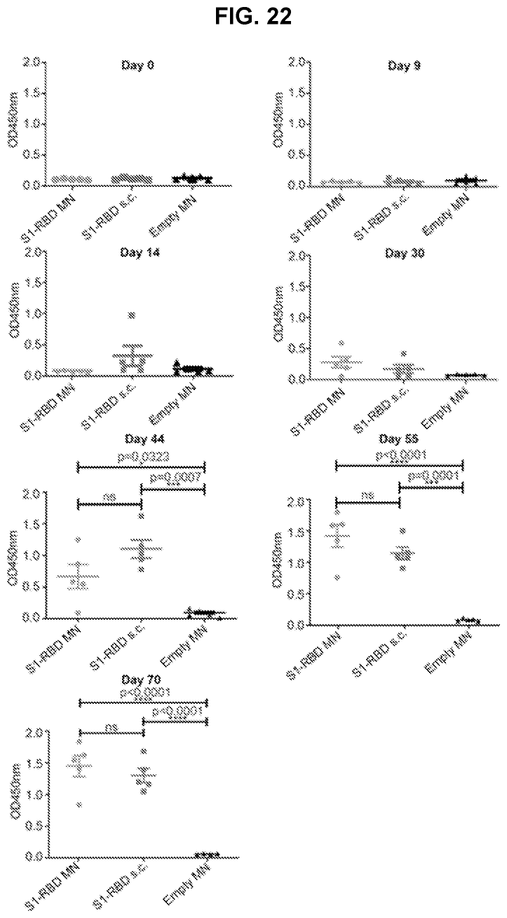

The development of antibodies against the S1-RBD protein over time was recorded from serum samples of vaccinated rats using ELISA. Antigen-specific IgG responses in S1-RBD-MN rats were highly similar to those of S1-RBD-s.c rats receiving multiple bolus subcutaneous injections, with no statistically significant difference identified (FIG. 18C-D and FIG. 22). The end-point binding antibody titer increased to 10⁴-10⁵, which is comparable to the reported range for those values obtained after vaccination of rodents with commercial COVID-19 vaccines (e.g., Moderna™). Meanwhile, the negative-control group receiving empty PLGA MNs exhibited no measurable immune response. Furthermore, to investigate the functionality of the induced antibodies, serum samples from vaccinated rats were analyzed for neutralizing activities against two authentic SARS-CoV-2 strains (ancestral US-WA1/2020 and delta B.1.617.2) using the plaque reduction neutralization test (PRNT). The rats immunized with S1-RBD loaded single-administration MNs developed robust neutralizing antibodies against both SARS-CoV-2 strains relative to background activity in unvaccinated animals. Neutralizing titers trended higher in S1-RBD-MN vaccinated animals in comparison to animals receiving multiple subcutaneous injections of the same antigen, though these differences were not statistically significant (FIG. 18E-F). Likely, the vaccine MNs enhanced immunogenicity through stimulation of professional antigen presenting immune cells (i.e., Langerhans's cells) in the dermal layer of the skin. On the other hand, serum from the unvaccinated control group displayed modest neutralization capacity (although some certain level of neutralizing activity is still naturally present). Adjuvants were not used in this study so that the antibody titters were not extremely high. However, the results presented herein clearly demonstrate the immunogenic efficacy of the single-administration stabilized-vaccine MN platform, comparable to that obtained by multiple injections of the fresh vaccine antigens. These MNs again offer unique significant advantages of being painless and easy-to-use while avoiding the needs of cold chain requirement and repeated administrations.

New approaches to thermally stabilize a protein vaccine against SARS-COV-2 viruses have been developed, and these vaccines have been incorporated into a recently developed single-administration multi-burst release MN platform via a novel high-throughput single-step antigen-loading process. The MNs are capable of releasing stabilized protein antigens at specific predetermined timepoints with only a single-time skin application, effectively mimicking the immunogenic effect of multiple bolus vaccine injections. This platform was successfully utilized to deliver a highly relevant protein antigen, the S1-RBD protein of SARS-CoV-2, which triggers a significant neutralizing antibody response in a rat model.

The inclusion of stabilizing agents allowed the S1-RBD MN patch to retain immunogenicity, even at an extreme temperature of 100° C. for at least 1 hr and after long-term storage (at least 4 months at 37° C.) outside of the normal cold-chain conditions typically required for vaccines, further demonstrating the utility of this platform in geographically isolated and austere environments. Loading of the MNs with recombinant SARS-CoV-2 S1-RBD protein induced both binding and functional neutralizing antibody responses in rats against different strains of SARS-CoV-2 virus, including the variant of concern B.1.617.2 (Delta), thus demonstrating the potential of this platform to being a viable needle-free alternative to the traditional vaccination paradigms.

The single-administration burst-release MNs with stabilized protein antigens, presented herein, could revolutionize the vaccination process by avoiding painful, inconvenient injections, trained medical personnel, cold-chain facilities for vaccine storage, and the need of booster shots. For the future development and clinical translation of this technology, the addition of suitable adjuvants to the formulations may help to boost the observed immune response.

Additionally, future studies may assess the activity of the MNs in large animal models (e.g., pigs or monkeys with immune systems and skin properties more similar to those of human) with larger sample sizes. Furthermore, continued improvements on the fabrication processes, especially product streamlining and automation, could enable the mass production of the MN patch in response to surge events and expedite the immunization of populations against SARS-CoV-2 variants or potential future viruses in pandemics. These single-administration MNs with stabilized protein antigens and a high-throughput method for antigen loading, as presented herein, could be a key solution to the success of global immunization campaigns against highly contagious viruses and infectious diseases by enabling an easy-to-use, easy-to-distribute, and painless vaccine delivery platform with built-in programmable booster release.

### Example 12

### A Single-Administration Long-Acting Antibody Microarray Platform with an Ultra-High Loading Capacity and Multiple Burst Releases of Thermally Stabilized Antibodies

Monoclonal antibodies (mAbs) are being explored as partners for long acting antiretrovirals used in prevention and treatments of many dangerous viral diseases such as the human immunodeficiency virus (HIV). Challenges associated with stability, frequent dosing requirement, and patient compliances of current antibody therapies require development of stabilized antibody formulations and a suitable delivering vehicle with satisfying loading dose, controlled released kinetics, bioavailability, and shelf-life requirements.

To this end, presented herein is a new strategy to create a single-administration long-acting antibody-delivery microarray (MA) patch which can carry a high-dosage of thermally stabilized mAb. The MA can be fully embedded into the skin via a single-time administration to deliver multi-doses of the same mAb or different mAbs over a long period; thus, sustaining the desired mAb concentrations in systemic circulation. Formulations with excipients could stabilize nearly 100% of human Ig (an antibody model) at temperatures up to 100° C. for at least 1 hr and at average human body temperature (37° C.) for up to 90 days. Furthermore, the MA patch loaded with b12 antibody—a human monoclonal antibody clinically used for HIV treatment—and excipients maintained its anti-viral activity against HIV after heat exposure. Finally, in vivo release studies of human Ig antibodies in rats have successfully provided a proof-of-concept for using this single-administration multi-burst release MA system to maintain suitable mAb plasma concentration within the therapeutic window. The MA patches also demonstrate the capability to co-deliver mixture of antibodies, which will expand protection effectiveness against many viral infections, including HIV.

Monoclonal antibodies are an important class of therapeutic agents that have been used to treat a wide variety of diseases such as cancer, autoimmune disorders, and other infectious diseases. More than 100 mAb therapeutics have been approved by the regulatory authorities for treatment of human immunodeficiency virus (HIV), COVID-19, Alzheimer's, etc. Many others are actively under clinical development. Specifically, potent broadly neutralizing antibodies (bNAbs) have emerged as potential alternatives or adjuvant to antiretroviral therapy (ART) in treating HIV. Although combination ART is highly effective, it requires lifelong medication. bNAbs uniquely target and bind to the HIV envelope as the virus seeks to enter new target cells or be expressed from previously infected cells. Besides suppressing the virus, bNAbs may induce additional T-cell immunomodulatory effects as seen for other forms of immunotherapy. As such, antibody treatments are being explored as partners for long acting antiretrovirals for use in therapy or prevention of HIV infection. Additionally, an administration of bNAbs mixture targeting different neutralizing epitopes could expand protection effectiveness. Still, the delivery of antibodies into human body faces many challenges that have yet to be overcome.

Bioavailability and efficacy of therapeutic antibodies critically depend on their circulating plasma half-lives which are usually short and difficult to control. In order to achieve high and persistent serum levels of antibodies for optimal efficacy, the mAb therapies typically require frequent longitudinal administrations of relatively large doses (sub-grams or grams). Intravenous infusion remains the most popular systemic delivery of choice. However, it involves medical facilities, pain, discomfort, high costs, repeated travels, and trained personnel to administer doses like other hypodermic injections, which cause stress on patients and an economic burden on the health-care systems, especially in resource-constrained areas. Designing minimally invasive, controlled-release, and easy-to-administer delivery vehicles for mAb therapies offer new possibilities to overcome aforementioned issues. Still, there has been little success; some attempts include the use of non-degradable poly(ethylene-co-vinylacetate) (EVAc, for the sustained release of antibodies), hyaluronic acid scaffolds, glycol chitosan and oxidized alginate hydrogels.

Protein-based therapeutic agents frequently suffer from formulation challenges when packaged in delivery vehicles. And antibodies are no exception. Formulations of antibodies in polymeric vehicles often include the use of organic solvents or thermal processing which are deleterious to protein structure and function. Limited studies and no FDA approved products are available for controlled and sustained release of antibodies from degradable biopolymers. These current controlled-release challenges associated with stability, dosing requirement, and patient compliances for mAb therapies could be resolved by developing stabilized antibody formulations and a suitable delivering vehicle with satisfying loading dose, released kinetics, bioavailability, and shelf-life requirements.

Microneedle array (microarray; MA) patches have emerged as an attractive platform to administer therapeutic agents into the body circulation bypassing stratum corneum barrier of the skin in a minimally invasive manner. Delivering therapeutic agents through transdermal route allows steady plasma concentrations to eliminate hepatic first pass metabolism. Unlike regular hypodermic needles, MAs painlessly pierce the skin by passing 10-15 μm through the nerve-free layer of the skin. The MA creates micro-dimensional pathways to transport its payload with high-localization, correct-targeting, and controlled-release. MA is self-administrable and often does not generate biohazard sharp wastes; thus, enhancing patient compliances, reducing healthcare logistics and avoiding the environment problem from the disposal of sharp needles.

Despite many favorable characteristics of MA, there has been very few successes in employing MA for mAb therapies due to (1) the need of repeated MA patch application over a long period to sustain therapeutic level of mAb in the plasma, (2) the limitation of loading capacity of the MA and (3) the need to thermally stabilize the mAb against heat. To elaborate, with the relatively large dose requirement in mAb therapies (hundreds of milligrams to grams), the small size of the MA presents an important obstacle while the most common drug-loading method using liquid-casting onto a MA mold usually results in very low loading capacity and efficacy; Only few hundreds of micrograms to less than 1 mg of the cargos can be loaded into a 1×1 cm² patch MA. Additionally, the process of MA manufacturing (e.g., micro-molding) involves the use of high temperatures which can be up to 60-80° C. and is thus detrimental to the heat-labile proteins like antibodies. Furthermore, most MAs are currently designed only for an instantaneous release of the antibodies right after the administration. Thus, they need to be stored at a low temperature and repeatedly applied over a long period to sustain the therapeutic mAb level inside the plasma.

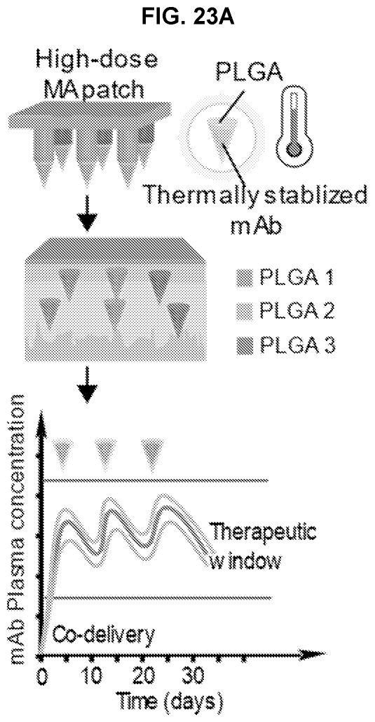

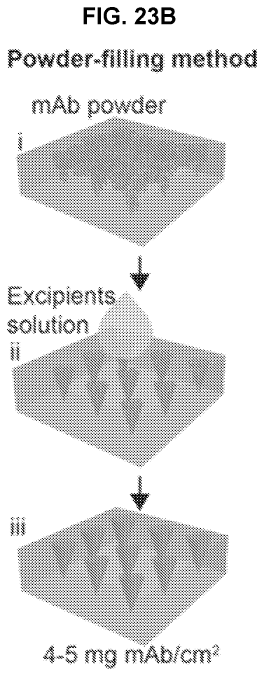

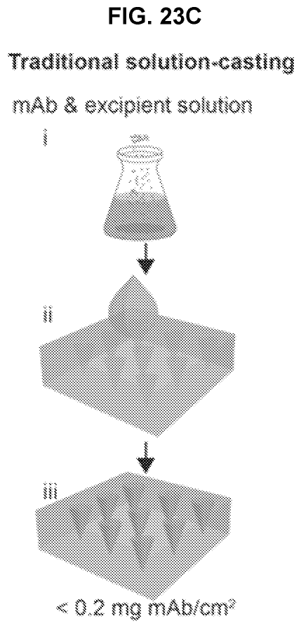

To overcome these problems, presented herein is a new strategy to create a single-administration long-acting antibody MA platform which can be loaded with a large amount of thermally stabilized mAb and fully embedded into the skin to repeatedly deliver the mAb over a long period, thus sustaining the desired mAb therapeutic concentration in systemic circulation (FIG. 23A). A new powder loading technique was first introduced, which could increase up to 20-fold loading amount of human Ig (an antibody model) in the MA patch compared to the conventional solution casting technique. Secondly, by using formulations with trehalose and sucrose, nearly 100% of the human Ig could be stabilized at temperatures up to 100° C. for at least 1 hr and at an average human body temperature (37° C.) for up to 4 months. The possibilities to co-deliver different kinds of human Ig at the same time via MA patches is also demonstrated. This further benefits the mAb therapy in the case of HIV treatment which prefers administration of bNAbs mixture instead of a single bNAb. The MA loaded with high and consistent amount of stabilized human Ig can be easily inserted into the skin in a minimally invasive manner and cause no significant skin irritations. Furthermore, by employing a previously reported fully embedded core-shell MA system, multiple longitudinal doses of human Ig could be delivered via a single-time skin insertion; thus, maintaining required therapeutic levels of antibodies in the systemic circulation. The single-administration long-acting antibody MA platform, presented herein, could significantly (1) enhance the patient compliance via the painless single-time (self-) administration and (2) increase the therapeutic antibody efficacy via the repeated release of stabilized mAb inside the body. This MA platform could offer a great impact on the field of antibody therapies/prophylaxis to treat and prevent many dangerous diseases like HIV, cancers, auto-immune, and other infectious/viral diseases.

### Fabrication of the Core-Shell Microneedle Array (MA) Patch

### Powder-Filling Technique

The mold for MA fabrication was first made by pouring and subsequently curing polydimethylsiloxane (PDMS) (SYLGARD™ 184 Silicone Elastomer Kit, The Dow Chemical Company, USA) elastomer onto a silicon wafer patterned with positive MA master structure (Nanoscribe GmbH, Germany). There was an array of 15×15 MNs in a 1×1 cm² patch with each MA height of 600 μm and base diameter of 300 μm. An amount of lyophilized human Ig powder (Millipore Sigma, USA) was weighed and filled into empty PDMS mold. The powders were then compressed under vacuum between two glass slides. The filling process could be repeated several times until all of the powder had been loaded in the PDMS mold. Because of the natural electrostatic charge of the powder, the filling process was conducted under neutralized static environment (Ionizing Air Bar 20 cm Anti-static Ionizing Bar, YUCHENGTECH, China). An excipient solution of sugars or water-soluble polymers of interest was later casted on top of the PDMS to provide a connected matrix for the powder. Finally, the residual excipient layer on top of the PDMS mold was removed and the MAs were transferred onto a sacrificial/supporting layer (i.e., PLGA film) using either heat-assisted micro-transferring.

### Core-Shell MA Patch Fabrication

The core-shell MA patch (with two delay burst release at days 10 and 21) manufacturing employs previously reported methods of micro-molding, 3D layer-by-layer assembly, and heat sintering. Briefly, four main components of the core-shell MNA including a polymeric shell, a core, a cap, and a supporting array were first prepared separately. A Poly(D,L-lactide-co-glycolide) (PLGA) (Millipore Sigma and Polysciences, Inc., USA) films (PLGA 50:50 acid-terminated 30 kDa and PLGA 50:50 60 kDa ester-terminated) were compression-molded into respective PDMS molds in the vacuum oven for 20-30 minutes at PLGA glass transition temperatures to make the shell and the cap of the MA. The MA core loaded antibody preparation was carried out by the powder-filling technique described above. The excipient solutions of interest were made by dissolving Trehalose, Sucrose, Trehalose and Sucrose (TS), Polyvinylpyrrolidone (PVP), Sorbitol (Millipore Sigma) in MilliQ water at desired concentrations. The supporting array for the core-shell MA was made by a two-step process involving solution casting effervescent polymer solution (PVP K30, citric acid, sodium bicarbonate, and ethanol) and Polylactic Acid (PLA) polymer molding to provide a flexible backing layer. The supporting array for the immediate release MA at Day 0 was made by compression molding PLA film.

### Drug Loading Process and 3D Layer-by-Layer Assembly

The core-shell MA patch was assembled as described previously. The optical images of the MA patch were taken by the AmScope ME300TZC-2L-8M with a digital camera (AmScope Mu800).

### Antibody Stability Study

For excipient formulations screening, antibodies including rat IgG (Millipore Sigma), human IgG (Millipore Sigma), human IgA (Millipore Sigma), human IgG1 and IgG2 (Fitzgerald Industries International, USA) were mixed with respective excipients solution at different ratios and vacuum dried overnight with the presence of desiccant (mimicking the MA core fabrication). Samples were then exposed to heat for short-term stability screenings (1 hr) at 56° C., 70° C., and 100° C. Long-term stability study was conducted at 37° C. (body temperature) for 14 days, 28 days, 56 days, and 90 days.

ELISA was used to determine the antibody activity after heat exposure. Depending on the antibodies, suitable commercially available ELISA kits including IgG (Total) Human Uncoated ELISA Kit (ThermoFisher, USA) and IgG Subclass Human ELISA Kit (ThermoFisher, USA) were used. Data were normalized to stock antibodies. Plates were read at 450 nm on a BioTek Synergy HT plate reader using BioTek Gen5 (BioTek). Data are representative of an average of three sample replicates and normalized to stock or initial antibodies activity after vacuum drying.

Coomassie Blue-stained SDS-PAGE gels of human IgG under varying conditions were performed. Samples were provided as either 20 μg dried protein or stock solution and were resuspended in 20 μL Ultrapure Dnase and Rnase free water (ThermoFisher, USA). 20 μL of each sample was mixed with Laemmli Sample Buffer (Bio-Rad Laboratories, Inc. USA) at 1:1 v/v ratio and incubated at 90° C. for 10 min. Then, 20 μL of each sample was loaded per well (8-16% Mini-PROTEAN® TGX Stain-Free™ Protein Gels, Bio-Rad Laboratories, Inc. USA), and run for 20 min at 220 V (PowerPac Basic Power Supply, Bio-Rad Laboratories, Inc. USA). The gels were Coomassie Blue stained overnight and then destained using destaining buffer (Coomassie Brilliant Blue R-250 staining solution, Bio-Rad Laboratories, Inc. USA). The gels were imaged on a Gel Doc light imager (Bio-Rad Laboratories, Inc. USA).

### b12 Anti-Viral Activity In Vitro Assay

TZM-bl cells were plated in 96-wells plate (10⁴ cells per well). The following day, the cells were exposed to different concentrations of b12 or MA alone in presence of HIV-1BaL (5×10³ TCID₅₀). The antiviral activity was assessed by using the Bright-Glo Luciferase Assay System (Promega, USA) following the manufacturer's instructions. After 48 h of exposure, the cells were lysed with 100 μL of Glo Lysis buffer. Lysate 50 μL was transferred into a 96-well black microtiter plate, after which 50 μL of Bright-Glo assay reagent was added, and the luminescence was measured and expressed in relative luminescence units (RLU). The average percentage infection of HIV-1BaL in the wells exposed to the b12 or controls and HIV was calculated and compared to the control (cells exposed to medium and HIV only: 100% infection). Statistical analysis was performed in GraphPad Prism 8.0 (GraphPad).

### Pharmacokinetic Studies of Antibody-Loaded MA Patches

Female Wistar rats (4-5 animals per group, aged 8 weeks; Charles River Laboratories) were administered with human Ig loaded MA patch using a commercially available applicator (Micropoint Technologies Pte Ltd, Singapore) for 15 minutes. Sterile saline was given subcutaneously as a negative control group. Sequential blood collections were performed every day. The serum was then collected from whole blood by centrifugation at 2,000 RPM for 5 min and stored at −20 ° C. until use. ELISA were performed using commercially available kits IgG (Total) Human Uncoated ELISA Kit (ThermoFisher, USA) and IgG Subclass Human ELISA Kit (ThermoFisher, USA) on collected serum to determine the plasma concentrations.

ELISA were performed to assess rat anti-human IgG antibodies. hIgG were diluted to a final concentration of 5 μg/mL in 100 mM carbonate-bicarbonate buffer (pH 9.6) and coated overnight onto Immulon1 B plates (Thermo Fisher Scientific) at 100 μL per well. BSA was diluted in carbonate-bicarbonate buffer to an equivalent concentration and used as a negative control. The plates were washed with Tween-20 in PBS (PBST) 3 times and blocked for 1 hr at room temperature with 2 μg/mL bovine serum albumin (BSA) 0.05% PBST. As a primary antibody, 100 μL of serum samples from rats diluted at 1:100 in PBST was loaded into each well and incubated for 1 hour at room temperature. Wells were then washed three times with 100 μL of PBST before loading the secondary antibodies. Goat anti-rat IgG, HRP diluted at 1:1,000 in PBST (Thermo Fisher Scientific) was used as a secondary antibody (100pL). The samples were incubated for 1 hour at room temperature. All of the wells were then washed three times with 100 μL of PBST before development. 100 μL TMB substrate (SouthernBiotech) was added to each well. After 15 min, the reaction was stopped with TMB Stop Solution (SouthernBiotech). Plates were read at 450 nm on a BioTek Synergy HT plate reader using BioTek Gen5 (BioTek). Data are representative of an average of two sample replicates.

### Skin Irritation Assessment

The rats' skins were monitored, and optical images were taken after the MAs insertion and throughout the during of the studies. Three individuals blinded with animal group assignments were asked to score the severity of erythema, eschar, and edema. Zero (0) is the minimum score which indicates normal (no irritations) and four (4) is the maximum possible score which indicates the severe skin irritation. The scores were presented as the mean and standard error of the mean (SEM).

### High Loading Dose of Antibodies in the Core-Shell MN Patch Using a Powder-Filling Technique

The low loading capacity of MA due to the small size of MNs has been a major hurdle that limits its widespread implementations, especially in antibody therapies. The most popular method to fabricate drug-carried MA is casting a liquid formulation onto a MA mold using vacuum-assisted method or centrifugation. The efficacy of this method relies on solubility and dispersity of the therapeutic agents as well as their surface interactions with the MA mold. Additionally, as the solution evaporates, it suffers from the coffee-ring effect and leaves behind a large empty space in the MA structure that might not be subsequently filled in, significantly lowering the loading capacity. Another approach is to increase the size of each individual MA and overall size of the MA patch. Nevertheless, there are certain limits to those factors to avoid pain and discomfort to patients. One goal of the present disclosure is to maximize the loading capacity of the MA by developing a new powder-filling technique to optimize volume usage of the MA (FIG. 23B-C). Starting from a positive MA master mold patterned on a silicon wafer using a two-photon polymerization technique, a negative mold was replicated by pouring and subsequently curing PDMS elastomer onto the silicon wafer. In a 1×1 cm² MA patch, there was an array of 15×15 microneedles. Each microneedle in the MA had a height of 600 μm and a base diameter of 300 μm.

A predetermined amount of lyophilized human IgG (hIgG) powder was weighed and filled into the empty PDMS mold. The PDMS mold containing the powder was placed between 2 glass slides, compressed using binder clips and then put under vacuum. The filling process was repeated until all the powder had been loaded in the PDMS mold. Due to the electrostatic charge of the powder, the filling process was conducted with the assistance of anti-static ionizing bars and anti-static gun to create a neutralized static environment. An excipient solution of sugars or water-soluble polymers was later casted on top of the PDMS to provide a connected matrix for the powder.

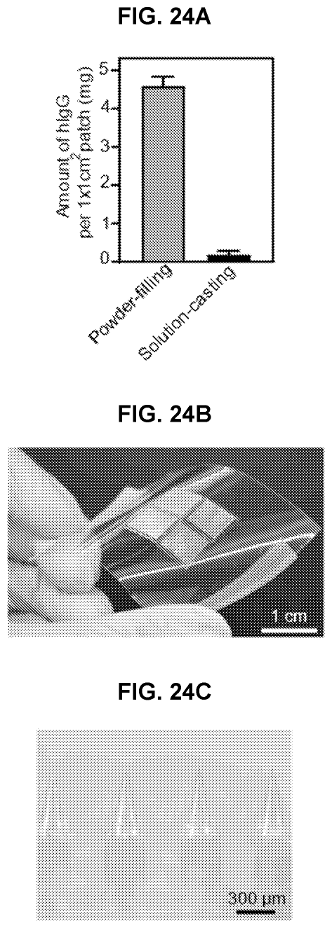

To study the effectiveness of this new powder-filling technique, the loading capacity was compared with the commonly used solution-casting technique in which the protein was mixed in excipient solutions at the highest concentration (i.e., solubility limit) and casted onto PDMS mold with the assistance of vacuum. Approximately 4.5 mg of hIgG loaded in 1×1 cm² MA patch from the powder-filling technique was achieved, which is 20-times higher than that of conventional solution-casting technique in the same patch size of 1 cm² (FIG. 24A). By filling the PDMS mold with powder form, the amount of the active ingredient occupied in a confined space of the MA patch was thus maximized and dramatically increased the loading capacity.

The 1×1 cm² MA patches can be considered as “pixels” and can be assembled together onto a single patch (e.g., 2×2 cm² with 4 pixels) to enhance the loading capacity by 4 times (or even more with more pixels), thus further increasing the maximal amount of mAb (FIG. 24B-C). Previous works have shown a patch size of 4 cm² or even up to 100 cm² could be used for human patient self-administration, allowing to boost the loading capacity up to the desired dosage of hundreds of milligrams. This ultrahigh loading capacity from the new powder-filling method has not been achieved for other available MA patches. It offers an effective solution for manufacturing MA patches, containing large amounts of drugs or proteins, extremely beneficial for not only mAb therapies, but also other applications requiring high antibody dosage.

### Strategy to Thermally Stabilize Antibodies

The improvement of thermal stability and eliminating cold-chain storage requirements benefit many classes of prophylactic and therapeutic agents including antibodies. When the protein is present in MA patches as a solid form, it avoids any mis-folding (with zero mobility) and thus has a more favorable thermostability profile compared to a liquid form. Yet, the structures of proteins could undergo stresses throughout the process of drying, molding, and storage in which they are susceptible to mis-folding and denaturation owing to the removal of their hydration shell. Establishing stabilized formulations that are specific to each protein is crucial to ensure stability and functionality during manufacturing, storage, and usage.

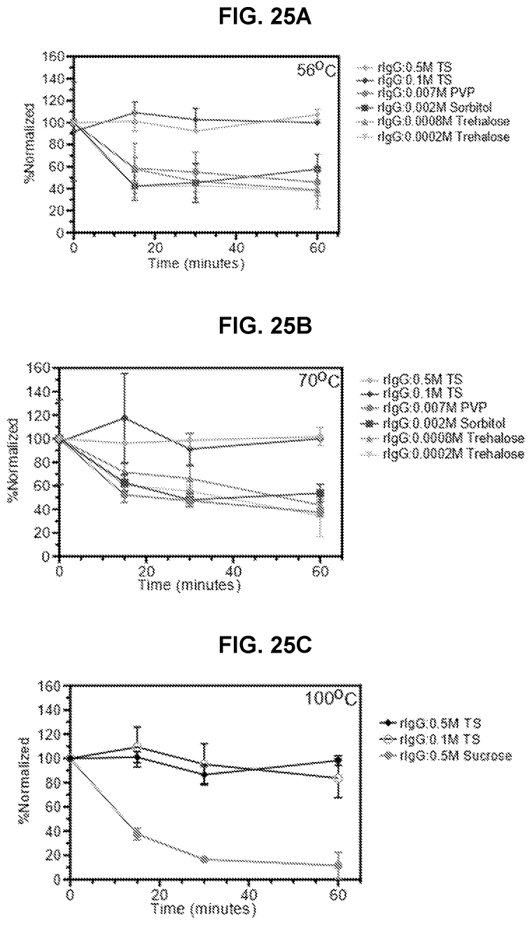

To this end, formulation screening was performed, and suitable excipients were identified which could successfully stabilize antibody proteins at different temperatures. As a preliminary study, several excipient candidates commonly used in pharmaceutical products as stabilizing agents were examined, including Polyvinylpyrrolidone (PVP), Sorbitol, Trehalose, and Sucrose formulated with Rat Immunoglobulin G (rIgG) antibody (FIG. 25A-C). The combination of Trehalose and Sucrose (TS) was determined as the most promising stabilizing excipient. This is because TS substitutes water molecules and directly interacts with proteins through hydrogen bonding, significantly reducing the protein mobility and preventing mis-folding. Highly viscous TS glass further prevents protein misfolding by entrapping protein conformations and slows molecular dynamics. The sugars could fill in void volumes and minimize proteins exposure to the environment.

Next, hIgG was chosen as an antibody model for continuation studies since they are the major antibody class used in the development of mAb therapies, including HIV treatment. The hIgG antibody was mixed with 0.5 M Trehalose-Sucrose (TS) solution at two ratios of 1:1 w/w, and 1:4 w/w and then vacuum dried with desiccants at room temperature until fully dried. To mimic temperatures that may be observed during the MA manufacturing process and quickly screen good formulations, the antibody samples were exposed to heat at 56° C., 70° C., and 100° C. for 1 hr (an accelerated protein degradation condition).

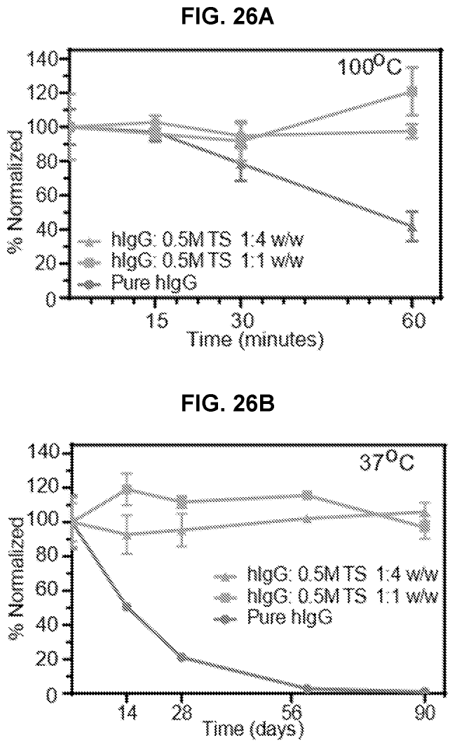

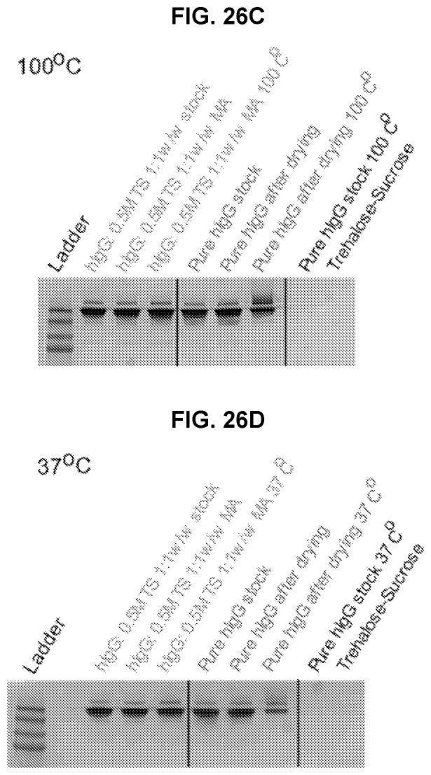

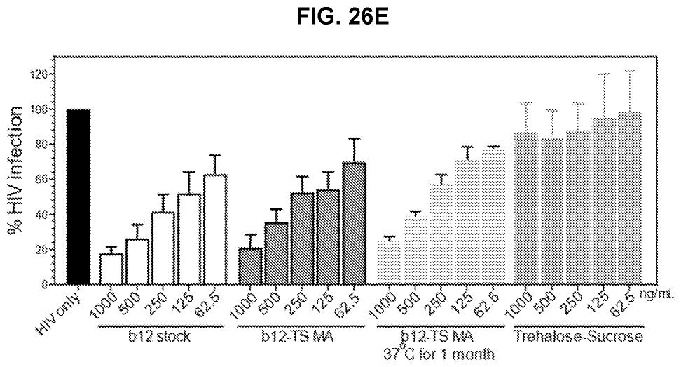

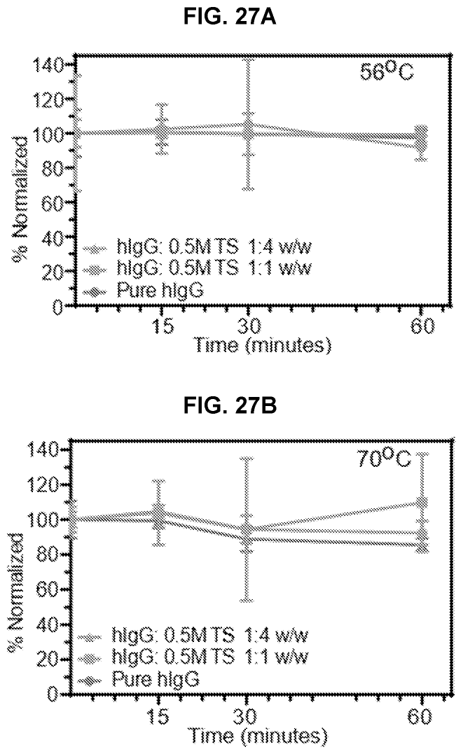

After this, a long-term stability study was conducted at 37° C. (approximate human body and hot environment temperature) for 14 days, 28 days, 56 days, and 90 days. ELISAs were used to assess functionality of hIgG after heat exposure. FIG. 26A-B and FIG. 27A-B illustrate the stabilization of hIgG using the various TS formulations at different thermal conditions. All formulations of hIgG with TS retained 100% antibody activity while pure hIgG, without excipients, presented a noticeable loss of activity following heat exposures. SDS-Page analysis further confirmed that the hIgG formulated with TS maintained its structural integrity after the heating (FIG. 26C-D). Moreover, incubation at 37° C. indicated that the formulations with TS at 1:1 w/w and 1:4 w/w ratios provided improved hIgG antibody long-term stability and outperformed those with pure antibody (i.e., without excipients). Clearly, the use of TS as excipients has provided an essential protection to hIgG after incubations at different temperatures for both short-term and long-term intervals.

To further confirm the benefit of TS for stabilizing antibodies, another study was carried out to assess the stability of b12 antibody—a human monoclonal antibody clinically used for HIV treatment—loaded inside the MA patch with TS and exposed to heat. MA patches containing b12 antibody and TS at 1:1 w/w (b12-TS) were incubated at 37° C. for one month. The anti-HIV activity of b12 loaded in MA patch was then measured using an in vitro functionality assay with real HIV-1BaL viruses in vitro. FIG. 26E shows that the neutralization effect of b12 antibody formulated with TS in the MA patch was comparable to the activity of stock b12 stored at −80° C. Importantly, the b12-TS MA patches were thermally stable outside recommended storage conditions for at least one month. The data also show no risk from the MA manufacturing process to the anti-viral activity of b12 antibody formulated with the TS excipients. These results together have strongly supported the benefit of TS to stabilize therapeutic antibodies for use in the MA platform.

### Pharmacokinetic Studies of Human Ig MNs

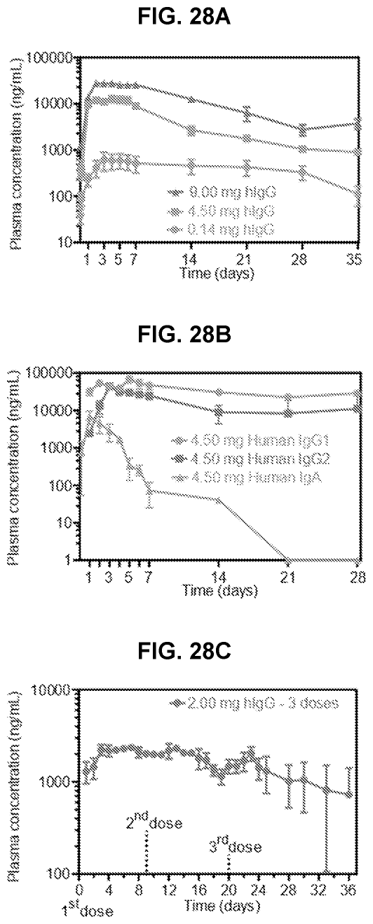

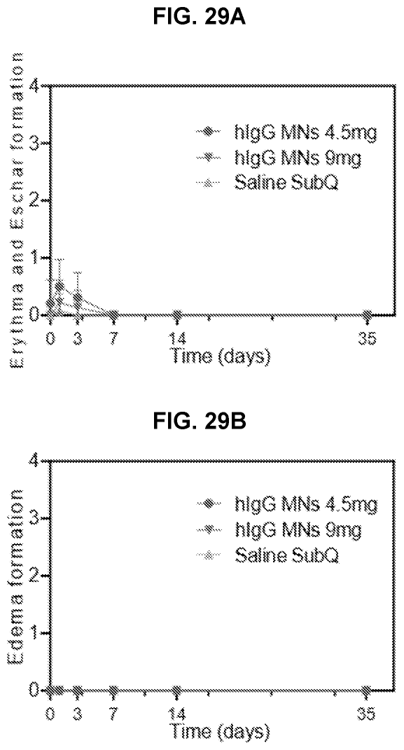

The next goal was to demonstrate the feasibility of using the MA patch to deliver high doses of antibodies in vivo by first showing the ability to control mAb dosage from the MA. Three sets of immediately dissolved MA patches were fabricated containing three different doses of 0.14 mg, 4.5 mg, and 9 mg hIgG. Since 4.5 mg was the maximum amount loaded in the 1×1 cm², the 9 mg group received two 1×1 cm² MA patches (or 1 patch of 1×2 cm²). The MA was fabricated on top of a Polylactic Acid (PLA) supporting array in order to allow the MA to be fully inserted into the skin effectively. The patches were then inserted on the skin of rats using a commercial MA applicator. Plasma concentrations of hIgG in circulation were collected and measured over time. The pharmacokinetic profiles are displayed in FIG. 28A. The mean serum antibody concentrations quantified by ELISA increased with the increase of mAb loaded inside the MA, showing a great control over the therapeutic mAb dose. Moreover, no skin irritations and inflammations were observed following MA administrations (FIG. 29A-B).

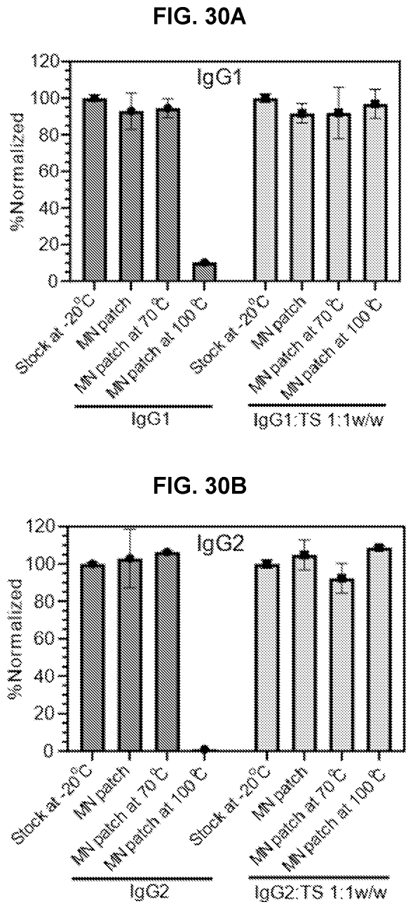

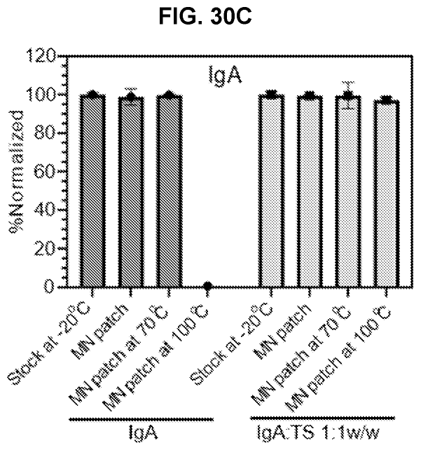

Co-administration of multiple bNAbs to enhance the effectiveness of mAb therapy for HIV and other diseases (e.g., cancers or auto-immune diseases) has been under active investigation. To test the possibility of the MA patch to simultaneously deliver different antibodies, another in vivo study was conducted in which a set of three MA patches was inserted into the skin of rats at the same time. Each patch (1×1 cm²) was loaded with 4.5 mg of human IgG1, human IgG2, or human IgA. Additionally, to ensure the MA manufacturing process did not affect activities of these antibodies, thermal stability assessments were performed again, showing the effectiveness of using trehalose-sucrose (TS) to stabilize the antibodies (FIG. 30A-C). After the MA administration, serum samples were collected and analyzed for plasma concentrations of each antibody using ELISA. In general, all three antibodies were detectable in serum at any timepoints suggesting the MA patches could deliver multiple antibodies as needed (FIG. 28B). The hIgG (human IgG1 and human IgG2) antibodies are known to exhibit a longer half-life than human IgA, which explains the observed higher plasma concentrations of the hIgG antibodies as compared to hIgA at a similar given dosage.

Antibodies require sustained systemic circulation of high doses in order to exert their therapeutic effects. Consequently, they are often administered repeatedly over time as multiple intravenous (IV) infusions, which causes significant burdens to healthcare facilities and patients due to the pain, cost, and inconvenience. To overcome these, a single-administration core-shell MA system was previously developed with built-in programmable delay multi-burst release of therapeutic cargos to mimic the effect of traditional multiple injections. In this system, each microneedle consists of an inner dry core containing biomolecules of interest, encapsulated in an outer biodegradable polymeric shell. Through tailoring properties such as molecular weight and compositions of the MA shell, made of Poly(D,L-lactide-co-glycolide) (PLGA), the release time of the MA can be precisely tuned to accommodate different release paradigms. In this regard, the core-shell MA system has been adapted for mAb therapies. The MAs after being embedded into the skin will automatically deliver multiple bursts of the encapsulated payloads after predictable periods of time, maintaining the concentration of antibodies within the preferable therapeutic window and eliminating the need for re-administrations. To demonstrate this idea, three sets of MAs were manufactured which include one set of stabilized hIgG MA for Day 0 release (i.e., immediate release) and two sets of core-shell MA patches using two different PLGA shells to provide delay burst releases at Day 10 and Day 21. According to the pharmacokinetic profiles of human IgG in FIG. 28A, the plasma level of human IgG seems to reduce after about 7-14 days, thus resulting in a need for another dosage after the same period (about 10 days on average). Therefore, three MA patches were designed to have burst releases at about days 0, 10, and 21. For clinical applications, depending on the half-life of the specific antibody and the required plasma concentration level, the release times of the MA could be readily modified to maintain the desired concentration within the therapeutic window. The manufacture of the single-administration core-shell multi-burst MA patch is based on previously work which uses micro-molding, 3D assembling, and heat sintering processes. To obtain burst release times at Day 10 and Day 21, two different shells were selected, made of PLGA 50:50 acid-terminated 30 kDa and PLGA 50:50 60 kDa ester-terminated according to previous work. Previous studies have extensively shown that the release time mainly depends on the chemistry (molecular weight and composition) of the PLGA shell without much influence from the cargos (i.e., different cargos with different molecular weights and sizes inside the same microneedle shell do not change the release time). Since PLGA degradation releases acidic byproducts that lower the local environmental pH, the pH stability of hIgG was also evaluated to ensure the protein maintained its potency at low pH (FIG. 31). Rats then received the three aforementioned hIgG loaded MA sets on their skins on Day 0 (i.e., single-time administration). Blood collections were performed and analyzed over time. By using the single-administration MA with pre-programmable multi-burst releases, the plasma concentration of hIgG antibody remained at approximately the same level with minimal fluctuations for 25 days (FIG. 28C). Owing to the second and third burst releases at days 10 and 21, there were increases of hIgG plasma level at those days. This release profile is completely different from that of an immediate-release IgG MA (i.e., only having the burst at day 0) which has a sharp decrease of the plasma IgG after 7-14 days of the administration (FIG. 28A). The result demonstrates the significance of having multiple burst release of the IgG after controllable periods, matching with the half-life of the antibody to sustain the therapeutic level of this agent inside the blood.

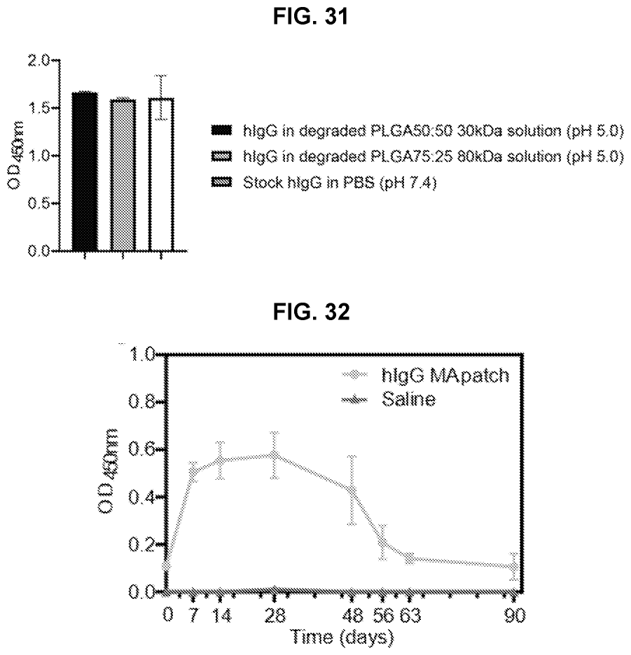

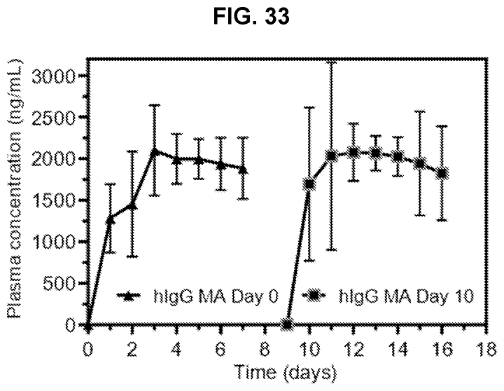

It is noted that there was a slight reduction on the plasma IgG peak level after the second and third doses (at days 10 and 21), compared to the antibody profile after the primary dose at day 0 (FIG. 28C). This is most likely due to the development of anti-human IgG antibody response in rats, especially after the second and third does (similar to the booster doses in vaccine). Indeed, FIG. 32 illustrates a strong anti-human IgG immune response from rats after receiving the one dose of human IgG via immediate release MA at Day 0 (i.e., the MA without PLGA shell). To confirm that the reduced plasma levels of hIgG after the second and third doses were not due to the thermal stability of hlgG or incomplete hIgG release from the core-shell MA patch, in vivo release experiments were further performed with two separate groups of animals. Group #1 received the hIgG MA patch with Day 0 release and group #2 received the core-shell hIgG MA patch with delay burst release at Day 10 only (i.e., without the primary dose at date 0). Both the groups received the same amount of hIgG (2 mg) from the MA patches. FIG. 33 shows that the plasma levels of hIgG released from the MA patches at Day 0 and Day 10 were similar, which strongly suggests that the reduced level of hIgG seen in the single-administration multi-burst MA patches after the 2nd and 3rd doses (FIG. 28C) is again due to the fact that more hIgG were neutralized by the developed anti-hIgG antibodies after the 2nd and 3rd dose in the rats. FIG. 33 also confirms that the core-shell MA patch provides a reliable delay burst release-time (at date 10) which is highly controllable by selecting an appropriate PLGA shell as described above.

Regardless of this minor issue of the anti-hIgG antibody development in the rat model (which is not expected in humans), the MA system presented herein offers an effective and novel approach for sustaining the plasma therapeutic antibody level via a single-time administration. It is demonstrated that antibodies can be stabilized against extreme, long-term heat exposure and a significant amount of the antibody can be loaded into a small MA patch. Moreover, the release time of the unique core-shell MA patches could be engineered to match desired dosing intervals of specific antibodies with their half-lives, thus further extending the systemic circulation time of any antibody of interest, which is crucial for antibody therapies. The success of (1) thermally stabilizing antibodies of interest, (2) obtaining a high mAb dose in a small MA patch, and (3) delivering multiple longitudinal mAb doses via a single-administration is a unique advantage of the present technology.

The new powder-filling technique has been introduced to overcome the low loading efficacy of traditional MA fabrication methods and create a new long-acting antibody therapy MA patch. Approximately 4.5 mg of hIgG was obtained in a 1×1 cm² MA patch from the powder-filling technique, which is 20-times higher than that of conventional solution-casting technique in the same patch size. The inclusion of suitable stabilizing excipients allowed the antibodies to retain their activities after the MA fabrication and a short-/long-term heat exposure at different temperatures (even up to 100° C.), outside of the normal refrigerated conditions typically required for protein-based biotherapeutics. Furthermore, the MA patch was successfully utilized to deliver high dosage and multiple types of human Ig in a rat model. Importantly, the previously reported core-shell PLGA microarray system was capable of sustaining the desired high dose of antibody (hIgG) concentration inside the animal blood over 25 days (a long period which has not been achieved before by other MA patches) with only a single-time skin insertion. This is due to the MA ability to repeatedly release the stabilized IgG at specific predetermined timepoints matching with the half-life of the antibody inside the blood.

Although a proof-of-concept has been achieved for the single-administration long-acting antibody MA patch, future studies would help to further develop this technology. These include the assessment on the sustainability of the long-term neutralization activities of the plasma antibody (delivered by the MA patch) against viral infections (e.g., HIV, SARS-COV-2). Studies using large animal models (e.g., pigs or monkeys with systemic circulations and skin properties more similar to those of human) and larger sample sizes could be performed to help confirm the effectiveness of the MA antibody patch. MA patch fabrication methods could also be further streamlined and scaled up with an automation process for a mass production and lower cost of the final products.

The results presented herein have demonstrated the excellent potential of this single-administration long-acting antibody MA patch which offers a needle-free alternative to the traditional hypodermic or intravenous injections for mAb therapies. This work is a significant step toward the development of a powerful antibody MA platform for many antibody therapies, including HIV, cancer, auto-immune diseases, and other infectious-disease treatment.

What is claimed:

1. A stabilized RNA vaccine composition, the composition comprising:
a complex of RNA with one or more cationic polymers; and
one or more cationic lipid entities.

2. The composition of claim 1, wherein the RNA comprises mRNA, miRNA, siRNA, tRNA, rRNA, or other RNA.

3. The composition of claim 1, wherein the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof.

4. The composition of claim 1, wherein the cationic polymer comprises protamine.

5. The composition of claim 1, wherein the one or more cationic lipid entities comprises liposomes or lipid nanoparticles.

6. The composition of claim 5, wherein the liposomes or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DM E), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof.

7. The composition of claim 1, further comprising one or more cryoprotectants.

8. The composition of claim 6, wherein the one or more cryoprotectants comprises one or more of trehalose, mannitol, sucrose, glucose, fructose, lactose, dextran, glycerol, ethylene glycol, propylene glycol, polyethylene glycol, or combinations thereof.

9. The composition of claim 1, further comprising one or more amino acids selected from lysine, arginine, histidine, glutamine, proline, glycine, threonine, or combinations thereof.

10. The composition of claim 1, wherein the complex of RNA with one or more cationic peptides has a size ranging from about 20 nm to about 500 nm.

11. The composition of claim 1, wherein the cationic lipid entity has a size ranging from about 20 nm to about 500 nm.

12. The composition of claim 1, wherein the stabilized RNA is thermally stable for at least about 2 months at about 37° C.

13. The composition of claim 1, wherein the stabilized RNA is thermally stable for at least about 2 hours at about 80° C.

14. A method for stabilizing RNA, the method comprising:
(a) forming a complex comprising the RNA with one or more cationic polymers;
(b) mixing the complex with one or more cationic lipid entities comprising liposomes or lipid nanoparticles to form a lipid mixture; and
(c) drying the lipid mixture under vacuum.

15. The method of claim 14, wherein the one or more cationic polymers comprise one or more of protamine, poly-lysine, poly-arginine, poly-histidine, amino group modified polycarbonate, amino group modified polypeptide, poly-β-amino esters, cationic cellulose, cationic dextran, cationic cyclodextrin, or combinations thereof.

16. The method of claim 14, wherein the liposome or lipid nanoparticles comprises one or more of dioleoyl-3-trimethylammonium propane (DOTAP), cholesterol, dimethylaminoethane-carbamoyl cholesterol hydrochloride (DC-Cholesterol), dioleoyl-3-trimethylammonium propane (DOTMA), distearoylphosphatidylcholine (DSPC), dipalmitoylphosphatidylcholine (DOPC), DSPE-PEG(2000) amine, Poly(ethylene glycol) dimethyl ether (PEG-DM E), (6Z,9Z,28Z,31Z)-heptatriacont-6,9,28,31-tetraene-19-yl 4-(dimethylamino)butanoate (DLin-MC3-DMA), dioleoylphosphatidylethanolamine (DOPE), or combinations thereof.

17. The method of claim 14, further comprising mixing the lipid mixture with one or more cryoprotectants comprising one or more of, trehalose, mannitol, sucrose, glucose, fructose, lactose, dextran, glycerol, ethylene glycol, propylene glycol, polyethylene glycol, or combinations thereof prior to drying the lipid mixture.

18. The method of claim 14, further comprising mixing the lipid mixture with one or more amino acids selected from lysine, arginine, histidine, glutamine, proline, glycine, threonine, or combinations thereof prior to drying the lipid mixture.

19. A stabilized RNA vaccine prepared by the method of claim 14.

20. Use of a stabilized RNA prepared by the method of claim 14 in the preparation of a vaccine medicament.

21. A stabilized vaccine agent composition, the composition comprising:
a vaccine agent; and one or more cryoprotectants comprising one or more of trehalose, mannitol, sucrose, glucose, fructose, lactose, dextran, glycerol, ethylene glycol, propylene glycol, polyethylene glycol, or combinations.

22. The composition of claim 21, wherein the one or more cryoprotectants have a concentration of about 0.3 M to about 1 M.

23. The composition of claim 21, wherein the vaccine agent and the one or more cryoprotectants are present in a ratio ranging from about 1:1 (v/v) to about 1:6 (v/v).

24. The composition of claim 21, wherein the vaccine agent is a protein, functional fragment thereof, or combination thereof.

25. The composition of claim 21, wherein the composition is dried by lyophilization, vacuum, or dessication.

26. The composition of claim 21, wherein the vaccine agent is thermally stable for at least about 4 months at about 37° C.

27. The composition of claim 21, wherein the vaccine agent is thermally stable for at least about 1 hour at about 100° C.

28. A method for preparing a stabilized vaccine agent core, the method comprising:
(a) mixing a vaccine agent with one or more cryoprotectants comprising one or more of trehalose, mannitol, sucrose, glucose, fructose, lactose, dextran, glycerol, ethylene glycol, propylene glycol, polyethylene glycol, or combinations thereof; and
(b) drying the mixture.

29. The method of claim 28, wherein the one or more cryoprotectants have a concentration of about 0.3 M to about 1 M prior to drying.

30. The method of claim 28, wherein the vaccine agent is mixed with the one or more cryoprotectants at a ratio ranging from about 1:1 (v/v) to about 1:6 (v/v) prior to drying.

31. The method of claim 28, wherein the vaccine agent is a protein, functional fragment thereof, or combination thereof.

32. The method of claim 28, wherein drying in step (b) is performed by lyophilization, vacuum, or dessication.

33. The method of claim 28, wherein drying in step (b) is performed for at least about 4 hours to about 24 hours.

34. A stabilized vaccine core prepared by the method of claim 28.

35. Use of a stabilized vaccine core prepared by the method of claim 28 in the preparation of a vaccine medicament.

36. A method for one-step loading a vaccine core into a microneedle, the method comprising:
(a) generating a core-shell microneedle assembly comprising a polymeric shell by compression-molding a film of poly(D,L-lactide-co-glycolide) (PLGA) into a silicone or poly(dimethylsiloxane) (PDMS) mold to form an assembly of microneedle shells, wherein the mold has an array of conical shaped cavities;
(b) generating a vaccine core array by combining a vaccine agent with a cryoprotectant and casting the vaccine core on a second poly(dimethylsiloxane) (PDMS) mold having the same array of conical shaped cavities as the microneedle mold and drying the vaccine core;
(c) transferring the vaccine core array onto a PLGA film;
(d) aligning the vaccine core array on the PLGA film with the microneedle assembly, thereby filling the cores in the microneedle shell assembly with the vaccine cores; and
(e) curing the microneedle assembly by vacuum heating to encapsulate the vaccine cores within the microneedles.

37. The method of claim 36, wherein generating a core-shell microneedle structure further comprises: removing an excess PLGA scum layer.

38. The method of claim 36, wherein the microneedles have a height of about 600 μm and a base diameter of about 300 μm.

39. The method of claim 36, wherein the vaccine cores have a height of about 400 μm and a diameter of about 200 μm.

40. A one-step loaded microneedle manufactured by the method of claim 36.

41. Use of a one-step loaded microneedle manufactured by the method of claim 36 to deliver a vaccine to a subject in need thereof.

42. A method for powder-filling a microneedle assembly, the method comprising:
(a) preparing a poly(dimethylsiloxane) (PDMS) mold having an array of conical shaped cavities on a silicon wafer;
(b) dispersing a powdered vaccine agent into the mold cavities;
(c) compressing the powdered vaccine agent into the mold cavities;
(d) repeating steps (b) and (c) until the microneedle cavities are filled;
(e) casting a layer of one or more sugars and water-soluble polymer over the filled cavities to generate a microneedle assembly; and
(f) drying the microneedle assembly.

43. The method of claim 42, wherein steps (b)-(d) are performed in a neutralized static environment.

44. The method of claim 42, wherein the vaccine agent loading capacity is about 20-fold greater than solution-casting core filling.

45. The method of claim 42, wherein the vaccine agent is a protein, a nucleic acid, or a combination thereof.

46. A filled microneedle assembly manufactured by the method of claim 42.

47. Use of a filled microneedle assembly manufactured by the method of claim 42 to deliver a vaccine to a subject in need thereof.

48. A method for manufacturing a microneedle assembly, the method comprising:
(a) generating a core microneedle assembly by:
filling a first silicone mold including a plurality of microneedle cavities with a copolymer,
spinning the first silicone mold to remove a first scum layer of the copolymer on the first silicone mold, and
generating a core in each of the copolymer-filled cavities;
(b) generating a therapeutic agent assembly by:
filling a plurality of cavities in a second silicone mold with a therapeutic agent,
spinning the second silicone mold to remove a second scum layer of the therapeutic agent, and
removing the molded therapeutic agent from the second mold and transferring the molded therapeutic agent to a substrate; and
(c) aligning the therapeutic agent assembly with the core microneedle assembly to thereby fill the cores in the core microneedle assembly with the therapeutic agent.

49. The method of claim 48, wherein the cavities in the first silicone mold and the second silicone mold are conical shaped.

50. The method of claim 48, wherein the copolymer is poly(D,L-lactide-co-glycolide) (PLGA).

51. The method of claim 48, further comprising generating the first silicone mold by pouring polydimethylsiloxane (PDMS) over a master structure and curing.

52. The method of claim 48, wherein the vaccine agent is a protein or a nucleic acid.

53. The method of claim 48, further comprising generating a cap assembly by:
(a) filling a plurality of cavities in a third silicone mold with a copolymer to form a plurality of caps;
(b) spinning the third silicone mold to remove a third scum layer of the copolymer on the third silicone mold;
(c) removing the molded caps from the third mold and transferring the molded caps onto the core microneedle assembly; and
(d) aligning the cap assembly with the core microneedle assembly to thereby cover the cores in the core microneedle assembly with the caps.

54. The method of claim 48, further comprising applying heat to bond the cap assembly to the core microneedle assembly.

55. The method of claim 48, further comprising coating a supporting array with a water-soluble polymer and contacting the supporting array with the cap assembly.

56. The method of claim 48, further comprising applying heat to bond the supporting array to the cap assembly for removing the microneedle assembly from the first silicone mold.

57. A microneedle device, manufactured by the method of claim 48.

58. Use of a microneedle device, manufactured by the method of claim 48 for the delivery of a vaccine to a subject in need thereof.

59. A pulsatile vaccine delivery system comprising:
a microneedle assembly including a plurality of microneedles filled with a vaccine agent;
the microneedles comprising biodegradable polymers; and
the microneedle assembly configured to release the vaccine agent at predetermined times with a predetermined amount of the vaccine agent while the microneedle assembly remains embedded in a subject.

60. The system of claim 59, wherein the biodegradable polymer degrades over time to release the therapeutic agent into the subject.

61. The system of claim 59, further comprising a computer processor in electronic communication with the microneedle assembly, the computer processor programmed with computer readable instructions to initiate the release of the vaccine agent.

62. The system of claim 61, further comprising an activator in electronic communication with the computer processor to initiate the release of the therapeutic agent.

63. The system of claim 59, wherein each of the microneedles in the plurality of microneedles is conical shaped.

64. The system of claim 59, wherein the vaccine agent is a protein or a nucleic acid.

65. The system of claim 59, wherein at least one of the microneedles in the plurality of microneedles includes a maximum height of about 600 μm.

66. Use of the pulsatile vaccine delivery system of claim 59 for the delivery of a vaccine to a subject in need thereof.

67. A vaccine delivery system comprising:
an assembly comprising:
(a) a substrate, and
(b) a plurality of microneedles extending from the substrate, each microneedle comprising:
a shell comprising a biodegradable polymer,
a core comprising a vaccine agent, and
a cap comprising a biodegradable polymer, the cap enclosing the core within the shell;
wherein the shell of each of the plurality of microneedles is selected to degrade at a predetermined time to release the vaccine agent therein into skin of a subject.

68. The vaccine delivery system of claim 67, wherein the biodegradable polymer comprises poly(D,L-lactide-co-glycolide) (PLGA) or poly-lactide acid (PLA).

69. The vaccine delivery system of claim 67, wherein the cap comprises PLGA or PLA.

70. The vaccine delivery system of claim 67, wherein the shell of a first subset of the plurality of microneedles is selected to degrade at a first time to release the therapeutic agent therein into skin of the subject, and wherein the shell of a second subset of the plurality of microneedles is selected to degrade at a second time to release the therapeutic agent therein into the skin of the subject.

71. The vaccine delivery system of claim 67, wherein the shell of a first subset of the plurality of microneedles is selected to degrade at a first time to release a first dose of the vaccine therein into skin of the subject, and wherein the shell of a second subset of the plurality of microneedles is selected to degrade at a second time to release a second dose of the vaccine therein into the skin of the subject.

72. The vaccine delivery system of claim 67, wherein the core of at least one of the microneedles includes a maximum diameter of about 200 μm and a maximum height of about 300 μm.

73. The vaccine delivery system of claim 67, wherein at least one of the microneedles in the plurality of microneedles includes a maximum height of about 600 μm.

74. Use of the vaccine delivery system of claim 67 for administering a vaccine to a subject in need thereof.

75. A method for administering a vaccine to a subject in need thereof, the method comprising:
contacting a vaccine delivery system with skin of the subject, the vaccine delivery system comprising:
an assembly comprising:
a substrate, and
a plurality of microneedles extending from the substrate, each microneedle comprising:
a shell comprising a biodegradable polymer,
a core comprising a vaccine agent, and
a cap comprising a biodegradable polymer, the cap enclosing the core within the shell;
wherein the shell of each of the plurality of microneedles is selected to degrade at a predetermined time to release the vaccine agent therein into the skin of the subject.

76. The method of claim 75, wherein the vaccine agent is a protein or a nucleic acid.

77. The method of claim 75, wherein the shell of a first subset of the plurality of microneedles is selected to degrade at a first time to release the therapeutic agent therein into the skin of the subject, and wherein the shell of a second subset of the plurality of microneedles is selected to degrade at a second time to release the therapeutic agent therein into the skin of the subject.

78. The method of claim 75, wherein the shell of a first subset of the plurality of microneedles is selected to degrade at a first time to release a first dose of the vaccine therein into the skin of the subject, and wherein the shell of a second subset of the plurality of microneedles is selected to degrade at a second time to release a second dose of the vaccine therein into the skin of the subject.
